SEQUENCE LISTING (134 sequences)

SEQ ID NO: 1 - protein, 21 aa, Artificial sequence
  Synthetic sequence A-chain of analogue of human insulin
GIVEQCCTSICSLYQLENYCX

SEQ ID NO: 2 - protein, 30 aa, Artificial sequence
  Synthetic sequence B-chain of analogue of human insulin
XVXQHLCGSHLVEALYLVCGERGFFYTPKX

SEQ ID NO: 3 - protein, 29 aa, Homo sapiens
HSQGTFTSDYSKYLDSRRAQDFVQWLMNT

SEQ ID NO: 4 - protein, 8 aa, Homo sapiens
KRNRNNIA

SEQ ID NO: 5 - protein, 37 aa, Homo sapiens
HSQGTFTSDYSKYLDSRRAQDFVQWLMNTKRNRNNIA

SEQ ID NO: 6 - protein, 30 aa, Artificial sequence
  Synthetic sequence Formula I of PCT/IB2012/000134
HXQGTFTSDYSXYLDXXAAXXFVXWLXXAX

SEQ ID NO: 7 - protein, 29 aa, Artificial sequence
  Synthetic sequence Formula IIa of PCT/IB2012/000134
HAQGTFTSDYSXYLDXXAAXXFVXWLLXA

SEQ ID NO: 8 - protein, 29 aa, Artificial sequence
  Synthetic sequence Formula IIb of PCT/IB2012/000134
HSQGTFTSDYSXYLDXXAAXXFVXWLLXA

SEQ ID NO: 9 - protein, 29 aa, Artificial sequence
  Synthetic sequence Formula IIIa of PCT/IB2012/000134
HAQGTFTSDYSXYLDSXAAXXFVXWLLXA

SEQ ID NO: 10 - protein, 29 aa, Artificial sequence
  Synthetic sequence Formula IIIb of PCT/IB2012/000134
HSQGTFTSDYSXYLDSXAAXXFVXWLLXA

SEQ ID NO: 11 - protein, 29 aa, Artificial sequence
  Synthetic sequence Formula IVa of PCT/IB2012/000134
HAQGTFTSDYSXYLDSXAAHXFVXWLLXA

SEQ ID NO: 12 - protein, 29 aa, Artificial sequence
  Synthetic sequence Formula IVb of PCT/IB2012/000134
HSQGTFTSDYSXYLDSXAAHXFVXWLLXA

SEQ ID NO: 13 - protein, 29 aa, Artificial sequence
  Synthetic sequence
HSQGTFTSDYSKYLDSKAAHDFVEWLLRA

SEQ ID NO: 14 - protein, 29 aa, Artificial sequence
  Synthetic sequence
HSQGTFTSDYSKYLDKKAAHDFVEWLLRA

SEQ ID NO: 15 - protein, 29 aa, Artificial sequence
  Synthetic sequence
HSQGTFTSDYSKYLDSKAAKDFVEWLLRA

SEQ ID NO: 16 - protein, 29 aa, Artificial sequence
  Synthetic sequence
HSQGTFTSDYSKYLDSKAAHDFVEWLKRA

SEQ ID NO: 17 - protein, 29 aa, Artificial sequence
  Synthetic sequence
HSQGTFTSDYSKYLDSKAAHDFVEWLLKA

SEQ ID NO: 18 - protein, 29 aa, Artificial sequence
  Synthetic sequence
HSQGTFTSDYSRYLDSKAAHDFVEWLLRA

SEQ ID NO: 19 - protein, 29 aa, Artificial sequence
  Synthetic sequence
HSQGTFTSDYSLYLDSKAAHDFVEWLLRA

SEQ ID NO: 20 - protein, 30 aa, Artificial sequence
  Synthetic sequence
HSQGTFTSDYSKYLDSKAAHDFVEWLLRAK

SEQ ID NO: 21 - protein, 30 aa, Artificial sequence
  Synthetic sequence
HSQGTFTSDYSKYLDSKAAHDFVEWLLSAK

SEQ ID NO: 22 - protein, 29 aa, Artificial sequence
  Synthetic sequence
HSQGTFTSDYSKYLDSKAAHDFVEWLKSA

SEQ ID NO: 23 - protein, 29 aa, Artificial sequence
  Synthetic sequence
HSQGTFTSDYSKYLDSKAAHDFVKWLLRA

SEQ ID NO: 24 - protein, 29 aa, Artificial sequence
  Synthetic sequence
HSQGTFTSDYSKYLDSCAAHDFVEWLLRA

SEQ ID NO: 25 - protein, 29 aa, Artificial sequence
  Synthetic sequence
HSQGTFTSDYSKYLDSCAAHDFVEWLLSA

SEQ ID NO: 26 - protein, 29 aa, Artificial sequence
  Synthetic sequence
HSQGTFTSDYSKYLDSKAACDFVEWLLRA

SEQ ID NO: 27 - protein, 29 aa, Artificial sequence
  Synthetic sequence
HSQGTFTSDYSKYLDKSAAHDFVEWLLRA

SEQ ID NO: 28 - protein, 29 aa, Artificial sequence
  Synthetic sequence
HAQGTFTSDYSKYLDSKAAHDFVEWLLSA

SEQ ID NO: 29 - protein, 30 aa, Artificial sequence
  Synthetic sequence
HAQGTFTSDYSKYLDSKAAHDFVEWLLSAK

SEQ ID NO: 30 - protein, 29 aa, Artificial sequence
  Synthetic sequence
HAQGTFTSDYSKYLDSKAARDFVAWLLRA

SEQ ID NO: 31 - protein, 29 aa, Artificial sequence
  Synthetic sequence
HAQGTFTSDYSKYLDSKAAKDFVAWLLRA

SEQ ID NO: 32 - protein, 29 aa, Artificial sequence
  Synthetic sequence
HAQGTFTSDYSKYLDSKAAHDFVEWLLRA

SEQ ID NO: 33 - protein, 29 aa, Artificial sequence
  Synthetic sequence
HAQGTFTSDYSKYLDSKAAHDFVEWLLKA

SEQ ID NO: 34 - protein, 29 aa, Artificial sequence
  Synthetic sequence
HAQGTFTSDYSKYLDSKAAKDFVAWLLSA

SEQ ID NO: 35 - protein, 29 aa, Artificial sequence
  Synthetic sequence
HAQGTFTSDYSKYLDSKAAHDFVAWLLKA

SEQ ID NO: 36 - protein, 29 aa, Artificial sequence
  Synthetic sequence
HAQGTFTSDYSKYLDKKAAHDFVAWLLRA

SEQ ID NO: 37 - protein, 29 aa, Artificial sequence
  Synthetic sequence
HAQGTFTSDYSRYLDSKAAHDFVEWLLSA

SEQ ID NO: 38 - protein, 29 aa, Artificial sequence
  Synthetic sequence
HAQGTFTSDYSKYLDSKAAHDFVKWLLSA

SEQ ID NO: 39 - protein, 29 aa, Artificial sequence
  Synthetic sequence
HAQGTFTSDYSLYLDSKAAHDFVEWLLSA

SEQ ID NO: 40 - protein, 29 aa, Artificial sequence
  Synthetic sequence
HAQGTFTSDYSKYLDSCAAHDFVEWLLSA

SEQ ID NO: 41 - protein, 29 aa, Artificial sequence
  Synthetic sequence
HAQGTFTSDYSKYLDSKAACDFVEWLLRA

SEQ ID NO: 42 - protein, 29 aa, Artificial sequence
  Synthetic sequence
HAQGTFTSDYSKYLDKKAAEDFVEWLLRA

SEQ ID NO: 43 - protein, 29 aa, Artificial sequence
  Synthetic sequence
HAQGTFTSDYSKYLDSKAAHDFVEWLLKA

SEQ ID NO: 44 - protein, 29 aa, Artificial sequence
  Synthetic sequence
HAQGTFTSDYSKYLDSKAAKDFVEWLLRA

SEQ ID NO: 45 - protein, 29 aa, Artificial sequence
  Synthetic sequence
HAQGTFTSDYSKYLDSKAAHEFVEWLLKA

SEQ ID NO: 46 - protein, 29 aa, Artificial sequence
  Synthetic sequence
HAQGTFTSDYSKYLDSKAAKEFVEWLLRA

SEQ ID NO: 47 - protein, 29 aa, Artificial sequence
  Synthetic sequence
HSQGTFTSDYSKYLDSKAAHDFVEWLLRA

SEQ ID NO: 48 - protein, 29 aa, Artificial sequence
  Synthetic sequence
HSQGTFTSDYSKYLDKKAAHDFVEWLLRA

SEQ ID NO: 49 - protein, 29 aa, Artificial sequence
  Synthetic sequence
HSQGTFTSDYSKYLDSKAAKDFVEWLLRA

SEQ ID NO: 50 - protein, 29 aa, Artificial sequence
  Synthetic sequence
HSQGTFTSDYSKYLDSKAAHDFVEWLKRA

SEQ ID NO: 51 - protein, 29 aa, Artificial sequence
  Synthetic sequence
HSQGTFTSDYSKYLDSKAAHDFVEWLLKA

SEQ ID NO: 52 - protein, 29 aa, Artificial sequence
  Synthetic sequence
HSQGTFTSDYSRYLDSKAAHDFVEWLLRA

SEQ ID NO: 53 - protein, 29 aa, Artificial sequence
  Synthetic sequence
HSQGTFTSDYSLYLDSKAAHDFVEWLLRA

SEQ ID NO: 54 - protein, 30 aa, Artificial sequence; identical to SEQ ID NO: 20
  Synthetic sequence

SEQ ID NO: 55 - protein, 30 aa, Artificial sequence; identical to SEQ ID NO: 21
  Synthetic sequence

SEQ ID NO: 56 - protein, 29 aa, Artificial sequence
  Synthetic sequence
HSQGTFTSDYSKYLDSKAAHDFVEWLKSA

SEQ ID NO: 57 - protein, 29 aa, Artificial sequence
  Synthetic sequence
HSQGTFTSDYSKYLDSKAAHDFVKWLLRA

SEQ ID NO: 58 - protein, 29 aa, Artificial sequence
  Synthetic sequence
HSQGTFTSDYSKYLDSCAAHDFVEWLLRA

SEQ ID NO: 59 - protein, 29 aa, Artificial sequence
  Synthetic sequence
HSQGTFTSDYSKYLDSCAAHDFVEWLLSA

SEQ ID NO: 60 - protein, 29 aa, Artificial sequence
  Synthetic sequence
HSQGTFTSDYSKYLDSKAACDFVEWLLRA

SEQ ID NO: 61 - protein, 29 aa, Artificial sequence
  Synthetic sequence
HSQGTFTSDYSKYLDKSAAHDFVEWLLRA

SEQ ID NO: 62 - protein, 29 aa, Artificial sequence
  Synthetic sequence
HAQGTFTSDYSKYLDSKAAHDFVEWLLSA

SEQ ID NO: 63 - protein, 30 aa, Artificial sequence; identical to SEQ ID NO: 29
  Synthetic sequence

SEQ ID NO: 64 - protein, 29 aa, Artificial sequence
  Synthetic sequence
HAQGTFTSDYSKYLDSKAARDFVAWLLRA

SEQ ID NO: 65 - protein, 29 aa, Artificial sequence
  Synthetic sequence
HAQGTFTSDYSKYLDSKAAKDFVAWLLRA

SEQ ID NO: 66 - protein, 29 aa, Artificial sequence
  Synthetic sequence
HAQGTFTSDYSKYLDSKAAHDFVEWLLKA

SEQ ID NO: 67 - protein, 29 aa, Artificial sequence
  Synthetic sequence
HAQGTFTSDYSKYLDSKAAHDFVEWLLRA

SEQ ID NO: 68 - protein, 29 aa, Artificial sequence
  Synthetic sequence
HAQGTFTSDYSKYLDSKAAHDFVEWLLKA

SEQ ID NO: 69 - protein, 29 aa, Artificial sequence
  Synthetic sequence
HAQGTFTSDYSKYLDSKAAKDFVAWLLSA

SEQ ID NO: 70 - protein, 29 aa, Artificial sequence
  Synthetic sequence
HAQGTFTSDYSKYLDSKAAHDFVAWLLKA

SEQ ID NO: 71 - protein, 29 aa, Artificial sequence
  Synthetic sequence
HAQGTFTSDYSKYLDKKAAHDFVAWLLRA

SEQ ID NO: 72 - protein, 29 aa, Artificial sequence
  Synthetic sequence
HAQGTFTSDYSRYLDSKAAHDFVEWLLSA

SEQ ID NO: 73 - protein, 29 aa, Artificial sequence
  Synthetic sequence
HAQGTFTSDYSKYLDSKAAHDFVKWLLSA

SEQ ID NO: 74 - protein, 29 aa, Artificial sequence
  Synthetic sequence
HAQGTFTSDYSLYLDSKAAHDFVEWLLSA

SEQ ID NO: 75 - protein, 29 aa, Artificial sequence
  Synthetic sequence
HAQGTFTSDYSKYLDSCAAHDFVEWLLSA

SEQ ID NO: 76 - protein, 29 aa, Artificial sequence
  Synthetic sequence
HAQGTFTSDYSKYLDSKAACDFVEWLLRA

SEQ ID NO: 77 - protein, 29 aa, Artificial sequence
  Synthetic sequence
HAQGTFTSDYSKYLDSKAAHDFVEWLLSA

SEQ ID NO: 78 - protein, 29 aa, Artificial sequence
  Synthetic sequence
HAQGTFTSDYSKYLDKKAAEDFVEWLLRA

SEQ ID NO: 79 - protein, 29 aa, Artificial sequence
  Synthetic sequence
HAQGTFTSDYSKYLDSKAAHDFVEWLLKA

SEQ ID NO: 80 - protein, 29 aa, Artificial sequence
  Synthetic sequence
HAQGTFTSDYSKYLDSKAAKDFVEWLLRA

SEQ ID NO: 81 - protein, 29 aa, Artificial sequence
  Synthetic sequence
HAQGTFTSDYSKYLDSKAAHEFVEWLLKA

SEQ ID NO: 82 - protein, 29 aa, Artificial sequence
  Synthetic sequence
HAQGTFTSDYSKYLDSKAAKEFVEWLLRA

SEQ ID NO: 83 - protein, 29 aa, Artificial sequence
  Synthetic sequence
HSQGTFTSDYSKYLDKKAAHDFVEWLLRA

SEQ ID NO: 84 - protein, 29 aa, Artificial sequence
  Synthetic sequence
HSQGTFTSDYSKYLDSKAAHDFVEWLKRA

SEQ ID NO: 85 - protein, 29 aa, Artificial sequence
  Synthetic sequence
HSQGTFTSDYSKYLDSKAAKDFVEWLLRA

SEQ ID NO: 86 - protein, 29 aa, Artificial sequence
  Synthetic sequence
HSQGTFTSDYSKYLDSKAAHDFVEWLLKA

SEQ ID NO: 87 - protein, 29 aa, Artificial sequence
  Synthetic sequence
HAQGTFTSDYSKYLDSKAAHDFVEWLLRA

SEQ ID NO: 88 - protein, 29 aa, Artificial sequence
  Synthetic sequence
HAQGTFTSDYSKYLDSKAARDFVAWLLRA

SEQ ID NO: 89 - protein, 29 aa, Artificial sequence
  Synthetic sequence Compound X of PCT/IB2012/000134
HAQGTFTSDYSKYLDSKAAHDFVEWLLSA

SEQ ID NO: 90 - protein, 29 aa, Artificial sequence
  Synthetic sequence
HAQGTFTSDYSKYLDSKAAHDFVEWLLKA

SEQ ID NO: 91 - protein, 29 aa, Artificial sequence
  Synthetic sequence
HAQGTFTSDYSKYLDSKAAHDFVEWLLKA

SEQ ID NO: 92 - protein, 29 aa, Artificial sequence
  Synthetic sequence
HAQGTFTSDYSKYLDSKAAHDFVEWLLKA

SEQ ID NO: 93 - protein, 29 aa, Artificial sequence
  Synthetic sequence
HSQGTFTSDYSKYLDSKAAHDFVEWLLRA

SEQ ID NO: 94 - protein, 29 aa, Artificial sequence
  Synthetic sequence
HAQGTFTSDYSKYLDSKAAKDFVAWLLRA

SEQ ID NO: 95 - protein, 29 aa, Artificial sequence
  Synthetic sequence
HAQGTFTSDYSKYLDSKAAHDFVEWLLSA

SEQ ID NO: 96 - protein, 29 aa, Artificial sequence
  Synthetic sequence
HAQGTFTSDYSKYLDSKAAHDFVEWLLSA

SEQ ID NO: 97 - protein, 29 aa, Artificial sequence
  Synthetic sequence
HAQGTFTSDYSKYLDSKAAHDFVEWLLSA

SEQ ID NO: 98 - protein, 29 aa, Artificial sequence
  Synthetic sequence
HAQGTFTSDYSKYLDSKAAHDFVEWLLSA

SEQ ID NO: 99 - protein, 29 aa, Artificial sequence
  Synthetic sequence
HSQGTFTSDYSKYLDSKAAHDFVEWLLSA

SEQ ID NO: 100 - protein, 29 aa, Artificial sequence
  Synthetic sequence
HSQGTFTSDYSKYLDSKAAHDFVEWLLSA

SEQ ID NO: 101 - protein, 29 aa, Artificial sequence
  Synthetic sequence
HSQGTFTSDYSKYLDSKAAHDFVEWLLSA

SEQ ID NO: 102 - protein, 29 aa, Artificial sequence
  Synthetic sequence
HSQGTFTSDYSKYLDSKAAHDFVEWLLSA

SEQ ID NO: 103 - protein, 29 aa, Artificial sequence
  Synthetic sequence
HSQGTFTSDYSKYLDSKAAHDFVEWLLSA

SEQ ID NO: 104 - protein, 29 aa, Artificial sequence
  Synthetic sequence
HSQGTFTSDYSKYLDSKAAHDFVEWLLSA

SEQ ID NO: 105 - protein, 29 aa, Artificial sequence
  Synthetic sequence
HAQGTFTSDYSKYLDSKAAHDFVEWLLSA

SEQ ID NO: 106 - protein, 29 aa, Artificial sequence
  Synthetic sequence
HAQGTFTSDYSKYLDSKAAHDFVEWLLSA

SEQ ID NO: 107 - protein, 29 aa, Artificial sequence
  Synthetic sequence
HAQGTFTSDYSKYLDSKAAHDFVEWLLSA

SEQ ID NO: 108 - protein, 29 aa, Artificial sequence
  Synthetic sequence
HAQGTFTSDYSKYLDSKAAHDFVEWLLSA

SEQ ID NO: 109 - protein, 29 aa, Artificial sequence
  Synthetic sequence
HAQGTFTSDYSKYLDSKAAHDFVEWLLSA

SEQ ID NO: 110 - protein, 29 aa, Artificial sequence
  Synthetic sequence
HAQGTFTSDYSKYLDSKAAHDFVEWLLSA

SEQ ID NO: 111 - protein, 29 aa, Artificial sequence
  Synthetic sequence
HAQGTFTSDYSKYLDSKAAHDFVEWLLSA

SEQ ID NO: 112 - protein, 29 aa, Artificial sequence
  Synthetic sequence
HAQGTFTSDYSKYLDKKAAHDFVEWLLSA

SEQ ID NO: 113 - protein, 29 aa, Artificial sequence
  Synthetic sequence
HAQGTFTSDYSKYLDSKAAKDFVEWLLSA

SEQ ID NO: 114 - protein, 29 aa, Artificial sequence
  Synthetic sequence
HAQGTFTSDYSKYLDSKAAHDFVKWLLSA

SEQ ID NO: 115 - protein, 29 aa, Artificial sequence
  Synthetic sequence
HAQGTFTSDYSKYLDSKAARDFVAWLLRA

SEQ ID NO: 116 - protein, 29 aa, Artificial sequence
  Synthetic sequence
HAQGTFTSDYSKYLDSKAAKDFVEWLLSA

SEQ ID NO: 117 - protein, 29 aa, Artificial sequence
  Synthetic sequence
HAQGTFTSDYSKYLDEKAAHDFVEWLLSA

SEQ ID NO: 118 - protein, 29 aa, Artificial sequence
  Synthetic sequence
HAQGTFTSDYSKYLDSKAAHEFVEWLLSA

SEQ ID NO: 119 - protein, 29 aa, Artificial sequence
  Synthetic sequence
HAQGTFTSDYSKYLDSKAAEDFVEWLLSA

SEQ ID NO: 120 - protein, 29 aa, Artificial sequence
  Synthetic sequence
HAQGTFTSDYSKYLDSKAAHDFVEWLLEA

SEQ ID NO: 121 - protein, 28 aa, Artificial sequence
  Synthetic sequence Formula V of PCT/IB2012/000134
HAQGTFTSDYSKYLDSXAAHDFVEWLLX

SEQ ID NO: 122 - protein, 28 aa, Artificial sequence
  Synthetic sequence
HAQGTFTSDYSKYLDSKAAHDFVEWLLS

SEQ ID NO: 123 - protein, 27 aa, Artificial sequence
  Synthetic sequence
HAQGTFTSDYSKYLDSKAAHDFVEWLL

SEQ ID NO: 124 - protein, 29 aa, Artificial sequence
  Synthetic sequence Formula VI of PCT/IB2012/000134
HAEGTFTSDYSKYLDSXAAHDFVEWLLSA

SEQ ID NO: 125 - protein, 29 aa, Artificial sequence
  Synthetic sequence
HAEGTFTSDYSKYLDSKAAHDFVEWLLSA

SEQ ID NO: 126 - protein, 29 aa, Artificial sequence
  Synthetic sequence
HAQGTFTSDYSKYLDSKAAHDFVEWLLSA

SEQ ID NO: 127 - protein, 21 aa, Artificial sequence
  Synthetic sequence A chain of human insulin analogue
XXXXXCCXXXCXXXXXXXXCX

SEQ ID NO: 128 - protein, 30 aa, Artificial sequence
  Synthetic sequence B chain of human insulin analogue
XAXEHLCXXXXXXXXXXXCXXXXXXXXXXX

SEQ ID NO: 129 - protein, 30 aa, Artificial sequence
  Synthetic sequence B chain of human insulin analogue
FVKQHLCGSHLVEALYLVCGERGFFYTPET

SEQ ID NO: 130 - protein, 30 aa, Artificial sequence
  Synthetic sequence B chain of human insulin analogue
FVKQHLCGSHLVEALYLVCGERGFFYIPKT

SEQ ID NO: 131 - protein, 30 aa, Artificial sequence
  Synthetic sequence B chain of human insulin analogue
FVKQHLCGSHLVEALYLVCGERGFFYTIKT

SEQ ID NO: 132 - protein, 24 aa, Artificial sequence
  Synthetic sequence A chain of human insulin analogue
GIVEQCCTSICSLYQLYQLENYCD

SEQ ID NO: 133 - protein, 21 aa, Artificial sequence
  Synthetic sequence A chain of human insulin analogue
GIVEQCCTSICSLYQLENYCN

SEQ ID NO: 134 - protein, 29 aa, Artificial sequence
  Synthetic sequence B chain of human insulin analogue
FVNQHLCGSHLVEALYLVCGERGFFYTPK

(19) United States
(12) Patent Application Publication
(10) Pub. No.: US 2014/0011733 A1
(43) Pub. Date: Jan. 9, 2014

(54) COMBINATION OF ACYLATED GLUCAGON ANALOGUES WITH INSULIN ANALOGUES

(71) Applicants: Keld Fosgerau, Vanlose (DK); Ditte Riber, Bronshoj (DK)

(72) Inventors: Keld Fosgerau, Vanlose (DK); Ditte Riber, Bronshoj (DK)

(73) Assignee: Zealand Pharma A/S, Glostrup (DK)

(21) Appl. No.: 13/980,087

(22) PCT Filed: Jan. 20, 2012
(86) PCT No.: PCT/IB12/00134

§ 371 Date: Sep. 24, 2013

Related U.S. Application Data
(60) Provisional application No. 61/434,698, filed on Jan. 20, 2011.

(51) Int. Cl. C07K 14/605; A61K 38/26; A61K 38/28

(52) U.S. Cl. CPC C07K 14/605; A61K 38/28; A61K 38/26
USPC 514/1.9; 514/6.5; 514/5.3; 514/11.7; 514/7.4; 514/7.2

(57) ABSTRACT

The invention relates to methods for treating metabolic disorders, including diabetes by using a combination of an acylated glucagon analogue and an insulin analogue. The invention also features a kit that includes an acylated glucagon analogue and an insuline analogue.

### FIELD OF THE INVENTION

The present invention relates to combinations of an acylated glucagon analogue with an insulin analogue and their medical use, for example, in the treatment of obesity and diabetes.

### BACKGROUND OF THE INVENTION

Obesity and diabetes are globally increasing health problems and are associated with various diseases, particularly cardiovascular disease (CVD), obstructive sleep apnea, stroke, peripheral artery disease, microvascular complications and osteoarthritis.

There are 246 million people worldwide with diabetes, and by 2025 it is estimated that 380 million will have diabetes. Many have additional cardiovascular risk factors including high/aberrant LDL and triglycerides and low HDL.

Cardiovascular disease accounts for about 50% of the mortality in people with diabetes and the morbidity and mortality rates relating to obesity and diabetes underscore the medical need for efficacious treatment options.

Preproglucagon is a 158 amino acid precursor polypeptide that is differentially processed in the tissues to form a number of structurally related proglucagon-derived peptides, including glucagon (Glu), glucagon-like peptide-1 (GLP-1), glucagon-like peptide-2 (GLP-2), and oxyntomodulin (OXM). These molecules are involved in a wide variety of physiological functions, including glucose homeostasis, insulin secretion, gastric emptying and intestinal growth, as well as regulation of food intake.

Glucagon is a 29-amino acid peptide that corresponds to amino acids 53 to 81 of pre-proglucagon and has the sequence His-Ser-Gln-Gly-Thr-Phe-Thr-Ser-Asp-Tyr-Ser-Lys-Tyr-Leu-Asp-Ser-Arg-Arg-Ala-Gln-Asp-Phe-Val-Gln-Trp-Leu-Met-Asn-Thr. Oxyntomodulin (OXM) is a 37 amino acid peptide which includes the complete 29 amino acid sequence of glucagon with an octapeptide carboxyterminal extension (amino acids 82 to 89 of pre-proglucagon, having the sequence Lys-Arg-Asn-Arg-Asn-Asn-Ile-Ala and termed “intervening peptide 1” or IP-1; the full sequence of human oxyntomodulin is thus His-Ser-Gln-Gly-Thr-Phe-Thr-Ser-Asp-Tyr-Ser-Lys-Tyr-Leu-Asp-Ser-Arg-Arg-Ala-Gln-Asp-Phe-Val-GM-Trp-Leu-Met-Asn-Thr-Lys-Arg-Asn-Arg-Asn-Asn-Ile-Ala), The major biologically active fragment of GLP-1 is produced as a 30-amino acid, C-terminally amidated peptide that corresponds to amino acids 98 to 127 of pre-proglucagon. Glucagon helps maintain the level of glucose in the blood by binding to glucagon receptors on hepatocytes, causing the liver to release glucose stored in the form of glycogen through glycogenolysis. As these stores become depleted, glucagon stimulates the liver to synthesize additional glucose by gluconeogenesis. This glucose is released into the bloodstream, preventing the development of hypoglycemia. Additionally, glucagon has been demonstrated to increase lipolysis and decrease body weight.

GLP-1 decreases elevated blood glucose levels by improving glucose-stimulated insulin secretion and promotes weight loss chiefly through decreasing food intake.

Oxyntomodulin is released into the blood in response to food ingestion and in proportion to meal calorie content. The mechanism of action of oxyntomodulin is not well understood. In particular, it is not known whether the effects of the hormone are mediated exclusively through the glucagon receptor and the GLP-1 receptor, or through one or more as-yet unidentified receptors.

Other peptides have been shown to bind and activate both the glucagon and the GLP-1 receptor (Hjort et al, Journal of Biological Chemistry, 269, 30121-30124, 1994) and to suppress body weight gain and reduce food intake (WO 2006/134340; WO 2007/100535; WO 2008/101017, WO 2008/152403, WO 2009/155257 and WO 2009/155258).

Stabilization of peptides has been shown to provide a better pharmacokinetic profile for several drugs. In particular addition of one or more polyethylene glycol (PEG) or acyl group has been shown to prolong half-life of peptides such as GLP-1 and other peptides with short plasma stability.

In WO 00/55184A1 and WO 00/55119 are disclosed methods for acylation of a range of peptides, in particular GLP-1. Madsen et al (J. Med. Chem. 2007, 50, 6126-6132) describe GLP-1 acylated at position 20 (Liraglutide) and provide data on its stability.

Stabilization of OXM by PEGylation and C-terminal acylation has also been shown to improve the pharmacokinetic profile of selected analogues in WO2007/100535, WO08/071,972 and in Endocrinology 2009, 150(4), 1712-1721 by Druce, M R et al.

It has recently been shown that PEGylation of glucagon analogues has a significant effect on the pharmacokinetic profile of the tested compounds (WO2008/101017) but also interferes with the potency of these compounds.

### SUMMARY OF THE INVENTION

In an first aspect, the invention features a combination of compounds for use in a method of treatment, a use, and a method for preventing or reducing weight gain; promoting weight loss; improving circulating glucose levels, glucose tolerance or circulating cholesterol levels; lowering circulating LDL levels; increasing HDL/LDL ratio; or treating a condition caused or characterized by excess body weight (e.g., obesity, morbid obesity, obesity-linked inflammation, obesity-linked gallbladder disease, obesity-induced sleep apnea, metabolic syndrome, pre-diabetes, insulin resistance, glucose intolerance, type 2 diabetes, type I diabetes, hypertension, atherogenic dyslipidaemia, atherosclerosis, arteriosclerosis, coronary heart disease, peripheral artery disease, stroke or microvascular disease). The combination of compounds for use in a method of treatment, a use, and a method employs administering to a mammalian (e.g., human) subject (e.g., having type I or type II diabetes) a combination of compounds including (a) a compound having the formula R¹—Z—R², where R¹ is H, C₁₋₄ alkyl, acetyl, formyl, benzoyl, or trifluoroacetyl; R² is OH or NH₂; and Z is a peptide having the formula I: His-X2-Gln-Gly-Thr-Phe-Thr-Ser-Asp-Tyr-Ser-X12-Tyr-Leu-Asp-X16-X17-Ala-Ala-X20-X21-Phe-Val-X24-Trp-Leu-X27-X28-Ala-X30; (I), where X2 is selected from Aib and Ser; X12 is selected from Lys, Arg, or Leu; X16 is selected from Arg and X; X17 is selected from Arg and X; X20 is selected from Arg, His, and X; X21 is selected from Asp and Glu; X24 is selected from Ala and X; X27 is selected from Leu and X; X28 is selected from Arg and X; X30 is X or is absent; where at least one of X16, X17, X20, X24, X27, X28, and X30 is X; and where each residue X is independently selected from the group consisting of Glu, Lys, Ser, Cys, Dbu, Dpr, and Orn (e.g., Lys, Glu, and Cys); where the side chain of at least one residue X is conjugated to a lipophilic substituent having the formula (i) Z¹, where Z¹ is a lipophilic moiety conjugated directly to the side chain of X; or (ii) Z¹Z², where Z¹ is a lipophilic moiety, Z² is a spacer, and Z¹ is conjugated to the side chain of X via Z²; and (b) an insulin analogue (e.g., insulin glulisine (Apidra™), insulin lispro (Humalog™), Degludec, LY2963016, LY2605541, pegylated insulin Lispro, insulin glargine (Lantus™, Glaritus, Basalin, Basalog, Glarvia, BIOD-620), insulin detemir (Levemir™) Humulin, Huminsulin, insulin isophane (Humulin N, Insulatard, Novolin N), insulin and insulin isophane (Humulin 70/30, Humulin 50/50, Mixtard 30, Actraphane™ HM), insulin degludec and insulin aspart (DegludecPlus/NN-5401), insulin aspart (Novolog), insulin aspart and insulin protamine (Novolog mix, Novolog mix 70/30), insulin (NN-1953, IN-105, HinsBet, Capsulin, Nasulin, Afrezza, ORMD-0801, SuliXen, Humulin R), insulin buccal (Oral-lyn) and hyaluronidase insulin (Analog-PH20)). The combination of (a) and (b) may be administered in amounts that together are effective. The component (a) and (b), respectively, may be administered within one month (e.g., within three, two, or one weeks; six, five, four, three, two, or one days; or 18, 12, 8, 6, 4, 3, 2, or 1 hours) of each other. The combination of compounds for use in a method of treatment, a use, and a method may prevent or reduce weight gain, may promote weight loss, or may improve circulating glucose levels.

In certain embodiments, X16 is selected from Glu, Lys, and Ser; X17 is selected from Lys and Cys; X20 is selected from His, Lys, Arg, and Cys; X24 is selected from Lys, Glu, and Ala; X27 is selected from Leu and Lys; and/or X28 is selected from Ser, Arg, and Lys. The peptide of formula I may include one or more of the following combinations of residues: X2 is Aib and X17 is Lys; X2 is Aib and X17 is Cys; X2 is Aib and X20 is Cys; X2 is Aib and X28 is Lys; X12 is Arg and X17 is Lys; X12 is Leu and X17 is Lys; X12 is Lys and X20 is Lys; X12 is Lys and X17 is Lys; X16 is Lys and X17 is Lys; X16 is Ser and X17 is Lys; X17 is Lys and X20 is Lys; X17 is Lys and X21 is Asp; X17 is Lys and X24 is Glu; X17 is Lys and X27 is Leu; X17 is Lys and X27 is Lys; X17 is Lys and X28 is Ser; X17 is Lys and X28 is Arg; X20 is Lys and X27 is Leu; X21 is Asp and X27 is Leu; X2 is Aib, X12 is Lys, and X16 is Ser; X12 is Lys, X17 is Lys, and X16 is Ser; X12 is Arg, X17 is Lys, and X16 is Glu; X16 is Glu, X17 is Lys, and X20 is Lys; X16 is Ser, X21 is Asp, and X24 is Glu; X17 is Lys, X24 is Glu, and X28 is Arg; X17 is Lys, X24 is Glu, and X28 is Lys; X17 is Lys, X27 is Leu, and X28 is Ser; X17 is Lys, X27 is Leu, and X28 is Arg; X20 is Lys, X24 is Glu, and X27 is Leu; X20 is Lys, X27 is Leu, and X28 is Ser; X20 is Lys, X27 is Leu, and X28 is Arg; X16 is Ser, X20 is His, X24 is Glu, and X27 is Leu; X17 is Lys, X20 is His, X24 is Glu, and X28 is Ser; X17 is Lys, X20 is Lys, X24 is Glu, and X27 is Leu; or X17 is Cys, X20 is Lys, X24 is Glu, and X27 is Leu. The peptide of formula I may contain only one amino acid of the type conjugated to the lipophilic substituent (e.g., only one Lys residue, only one Cys residue, or only one Glu residue, where the lipophilic substituent is conjugated to that residue). The peptide sequence of formula I may include one or more intramolecular bridges (e.g., a salt bridge or a lactam ring), for example, where the intramolecular bridge is formed between the side chains of two amino acid residues which are separated by three amino acids (e.g., between the side chains of residue pairs 16 and 20, 17 and 21, 20 and 24, or 24 and 26) in the linear amino acid sequence of formula I. The intramolecular bridge may involve a pair of residues selected from the group consisting of: X16 is Glu and X20 is Lys; X16 is Glu and X20 is Arg; X16 is Lys and X20 is Glu; X16 is Arg and X20 is Glu; X17 is Arg and X21 is Glu; X17 is Lys and X21 is Glu; X17 is Arg and X21 is Asp; X17 is Lys and X21 is Asp; X20 is Glu and X24 is Lys; X20 is Glu and X24 is Arg; X20 is Lys and X24 is Glu; X20 is Arg and X24 is Glu; X24 is Glu and X28 is Lys; X24 is Glu and X28 is Arg; X24 is Lys and X28 is Glu; and X24 is Arg and X28 is Glu.

In certain embodiments of any of the combinations of compounds for use in methods of treatment, uses, and methods described above, at least one of X16, X17, X20, and X28 is conjugated to a lipophilic substituent. X30 may be absent or X30 may be present and may be conjugated to a lipophilic substituent, for example, only one lipophilic substituent (e.g., at position 16, 17, 20, 24, 27, 28 or 30; position 16, 17 or 20, or at position 17) or exactly two lipophilic substituents, e.g., each at one of positions 16, 17, 20, 24, 27, 28, and 30 (e.g., at positions 16 and 17, 16 and 20, 16 and 24, 16 and 27, 16 and 28, 16 and 30, 17 and 20, 17 and 24, 17 and 27, 17 and 28, 17 and 30, 20 and 24, 20 and 27, 20 and 28, 20 and 30, 24 and 27, 24 and 28, 24 and 30, 27 and 28, 27 and 30, or 28 and 30).

In certain embodiments of any of the combinations of compounds for use in methods of treatment, uses, and methods described above, the compound has the formula: R¹—Z—R², where R¹ is H, C₁₋₄ alkyl, acetyl, formyl, benzoyl, or trifluoroacetyl; R² is OH or NH₂; and Z is a peptide having the formula IIa: His-Aib-Gln-Gly-Thr-Phe-Thr-Ser-Asp-Tyr-Ser-X12-Tyr-Leu-Asp-X16-X17-Ala-Ala-X20-X21-Phe-Val-X24-Trp-Leu-Leu-X28-Ala; (IIa); where X12 is selected from Lys, Arg, and Leu; X16 is selected from Ser and X; X17 is X; X20 is selected from His and X; X21 is selected from Asp and Glu; X24 is selected from Ala and Glu; X28 is selected from Ser, Lys, and Arg; and where each residue X is independently selected from the group consisting of Glu, Lys, and Cys; where the side chain of at least one residue X is conjugated to a lipophilic substituent having the formula (I) Z¹, where Z¹ is a lipophilic moiety conjugated directly to the side chain of X; or (ii) Z¹Z², where Z¹ is a lipophilic moiety, Z² is a spacer, and Z¹ is conjugated to the side chain of X via Z².

In other embodiments of the above combinations of compounds for use in methods of treatment, uses, and methods, the compound has the formula R¹—Z—R², where R¹ is H, C₁₋₄ alkyl, acetyl, formyl, benzoyl, or trifluoroacetyl; R² is OH or NH₂; and Z is a peptide having the formula IIb: His-Ser-Gln-Gly-Thr-Phe-Thr-Ser-Asp-Tyr-Ser-X12-Tyr-Leu-Asp-X16-X17-Ala-Ala-X20-X21-Phe-Val-X24-Trp-Leu-Leu-X28-Ala; (IIb); where X12 is selected from Lys, Arg, and Leu; X16 is selected from Ser and X; X17 is X; X20 is selected from His and X; X21 is selected from Asp and Glu; X24 is selected from Ala and Glu; X28 is selected from Ser, Lys, and Arg; and where each residue X is independently selected from the group consisting of Glu, Lys, and Cys; where the side chain of at least one residue X is conjugated to a lipophilic substituent having the formula (I) Z¹, where Z¹ is a lipophilic moiety conjugated directly to the side chain of X; or (ii) Z¹Z², where Z¹ is a lipophilic moiety, Z² is a spacer, and Z¹ is conjugated to the side chain of X via Z².

In particular embodiments, the compound has the formula R¹—Z—R², where R¹ is H, C₁₋₄ alkyl, acetyl, formyl, benzoyl, or trifluoroacetyl; R² is OH or NH₂; and Z is a peptide having the formula IIIa: His-Aib-Gln-Gly-Thr-Phe-Thr-Ser-Asp-Tyr-Ser-X12-Tyr-Leu-Asp-Ser-X17-Ala-Ala-X20-X21-Phe-Val-X24-Trp-Leu-Leu-X28-Ala; (IIIa); where X12 is selected from Lys And Arg; X17 is X; X20 is selected from His and X; X21 is selected from Asp and Glu; X24 is selected from Ala and Glu; X28 is selected from Ser, Lys, and Arg; and where each residue X is independently selected from Glu, Lys, and Cys; where the side chain of at least one residue X is conjugated to a lipophilic substituent having the formula (I) Z¹, where Z¹ is a lipophilic moiety conjugated directly to the side chain of X; or (ii) Z¹Z², where Z′ is a lipophilic moiety, Z² is a spacer, and Z¹ is conjugated to the side chain of X via Z².

In particular embodiments, the compound has the formula R¹—Z—R², where R′ is H, C₁₋₄ alkyl, acetyl, formyl, benzoyl, or trifluoroacetyl; R² is OH or NH₂; and Z is a peptide having the formula IIIb: His-Ser-Gln-Gly-Thr-Phe-Thr-Ser-Asp-Tyr-Ser-X12-Tyr-Leu-Asp-Ser-X17-Ala-Ala-X20-X21-Phe-Val-X24-Trp-Leu-Leu-X28-Ala; (IIIb); where X12 is selected from Lys and Arg; X17 is X; X20 is selected from His and X; X21 is selected from Asp and Glu; X24 is selected from Ala and Glu; X28 is selected from Ser, Lys, and Arg; and where each residue X is independently selected from Glu, Lys, and Cys; where the side chain of at least one residue X is conjugated to a lipophilic substituent having the formula: (i) Z¹, where Z¹ is a lipophilic moiety conjugated directly to the side chain of X; or (ii) Z¹Z², where Z′ is a lipophilic moiety, Z² is a spacer, and Z¹ is conjugated to the side chain of X via Z².

In other particular embodiments, the compound has the formula: R¹—Z—R², where R¹ is H, C₁₋₄ alkyl, acetyl, formyl, benzoyl, or trifluoroacetyl; R² is OH or NH₂; and Z is a peptide having the formula IVa: His-Aib-Gln-Gly-Thr-Phe-Thr-Ser-Asp-Tyr-Ser-X12-Tyr-Leu-Asp-Ser-X17-Ala-Ala-His-X21-Phe-Val-X24-Trp-Leu-Leu-X28-Ala; (IVa); where X12 is selected from Lys and Arg; X17 is X; X21 is selected from Asp and Glu; X24 is selected from Ala and Glu; X28 is selected from Ser, Lys, and Arg; where X is selected from the group consisting of Glu, Lys, and Cys; and where the side chain of X is conjugated to a lipophilic substituent having the formula (I) Z¹, where Z¹ is a lipophilic moiety conjugated directly to the side chain of X; or (ii) Z¹Z², where Z¹ is a lipophilic moiety, Z² is a spacer, and Z¹ is conjugated to the side chain of X via Z².

In still other particular embodiments, the compound has the formula R¹—Z—R², where R¹ is H, C₁₋₄ alkyl, acetyl, formyl, benzoyl, or trifluoroacetyl; R² is OH or NH₂; and Z is a peptide having the formula IVb: His-Ser-Gln-Gly-Thr-Phe-Thr-Ser-Asp-Tyr-Ser-X12-Tyr-Leu-Asp-Ser-X17-Ala-Ala-His-X21-Phe-Val-X24-Trp-Leu-Leu-X28-Ala; (IVb); where X12 is selected from Lys and Arg; X17 is X; X21 is selected from Asp and Glu; X24 is selected from Ala and Glu; X28 is selected from Ser, Lys, and Arg; where X is selected from the group consisting of Glu, Lys, and Cys; and where the side chain of X is conjugated to a lipophilic substituent having the formula (I) Z¹, where Z¹ is a lipophilic moiety conjugated directly to the side chain of X; or (ii) Z¹Z², where Z¹ is a lipophilic moiety, Z² is a spacer, and Z¹ is conjugated to the side chain of X via Z².

In any of the above combinations of compounds for use in methods of treatment, uses, and methods, the peptide Z may have the sequence HSQGTFTSDYSKYLDSKAAHDFVEWLLRA;

### HSQGTFTSDYSKYLDKKAAHDFVEWLLRA;

### HSQGTFTSDYSKYLDSKAAKDFVEWLLRA;

### HSQGTFTSDYSKYLDSKAAHDFVEWLKRA;

### HSQGTFTSDYSKYLDSKAAHDFVEWLLKA;

### HSQGTFTSDYSRYLDSKAAHDFVEWLLRA;

### HSQGTFTSDYSLYLDSKAAHDFVEWLLRA;

### HSQGTFTSDYSKYLDSKAAHDFVEWLLRAK;

### HSQGTFTSDYSKYLDSKAAHDFVEWLLRAK;

### HSQGTFTSDYSKYLDSKAAHDFVEWLKSA;

### HSQGTFTSDYSKYLDSKAAHDFVKWLLRA;

### HSQGTFTSDYSKYLDSCAAHDFVEWLLRA;

### HSQGTFTSDYSKYLDSCAAHDFVEWLLSA;

### HSQGTFTSDYSKYLDSKAACDFVEWLLRA;

### HSQGTFTSDYSKYLDKSAAHDFVEWLLRA;

### H-Aib-QGTFTSDYSKYLDSKAAHDFVEWLLSA;

### H-Aib-QGTFTSDYSKYLDSKAAHDFVEWLLSAK;

### H-Aib-QGTFTSDYSKYLDSKAARDFVAWLLRA;

### H-Aib-QGTFTSDYSKYLDSKAAKDFVAWLLRA;

### H-Aib-QGTFTSDYSKYLDSKAAHDFVEWLLRA;

### H-Aib-QGTFTSDYSKYLDSKAAHDFVEWLLKA;

### H-Aib-QGTFTSDYSKYLDSKAAKDFVAWLLSA;

### H-Aib-QGTFTSDYSKYLDSKAAHDFVAWLLKA;

### H-Aib-QGTFTSDYSKYLDKKAAHDFVAWLLRA;

### H-Aib-QGTFTSDYSRYLDSKAAHDFVEWLLSA;

### H-Aib-QGTFTSDYSKYLDSKAAHDFVKWLLSA;

### H-Aib-QGTFTSDYSLYLDSKAAHDFVEWLLSA;

### H-Aib-QGTFTSDYSKYLDSCAAHDFVEWLLSA;

### H-Aib-QGTFTSDYSKYLDSKAACDFVEWLLRA;

### H-Aib-QGTFTSDYSKYLDK( )KAAE( )DFVEWLLRA;

### H-Aib-QGTFTSDYSKYLDSKAAHDFVE( )WLLK( )A;

### H-Aib-QGTFTSDYSKYLDSKAAK( )DFVE( )WLLRA;

### H-Aib-QGTFTSDYSKYLDSK( )AAHE( )FVEWLLKA; or

### H-Aib-QGTFTSDYSKYLDSK( )AAKE( )FVEWLLRA.

In other embodiments, the peptide Z has the formula

### HSQGTFTSDYSKYLDS-K*-AAHDFVEWLLRA;

### HSQGTFTSDYSKYLD-K*-KAAHDFVEWLLRA;

### HSQGTFTSDYSKYLDSKAA-K*-DFVEWLLRA;

### HSQGTFTSDYSKYLDSKAAHDFVEWL-K*-RA;

### HSQGTFTSDYSKYLDSKAAHDFVEWLL-K*-A;

### HSQGTFTSDYSRYLDS-K*-AAHDFVEWLLRA;

### HSQGTFTSDYSLYLDS-K*-AAHDFVEWLLRA;

### HSQGTFTSDYSKYLDSKAAHDFVEWLLRA-K*;

### HSQGTFTSDYSKYLDSKAAHDFVEWLLSA-K*;

### HSQGTFTSDYSKYLDSKAAHDFVEWL-K*-SA;

### HSQGTFTSDYSKYLDSKAAHDFV-K*-WLLRA;

### HSQGTFTSDYSKYLDS-C*-AAHDFVEWLLRA;

### HSQGTFTSDYSKYLDS-C*-AAHDFVEWLLSA;

### HSQGTFTSDYSKYLDSKAA-C*-DFVEWLLRA;

### HSQGTFTSDYSKYLD-K*-SAAHDFVEWLLRA;

### H-Aib-QGTFTSDYSKYLDS-K*-AAHDFVEWLLSA;

### H-Aib-QGTFTSDYSKYLDSKAAHDFVEWLLSA-K*;

### H-Aib-QGTFTSDYSKYLDS-K*-AARDFVAWLLRA;

### H-Aib-QGTFTSDYSKYLDSKAA-K*-DFVAWLLRA;

### H-Aib-QGTFTSDYSKYLDSKAAHDFVEWLL-K*-A;

### H-Aib-QGTFTSDYSKYLDS-K*-AAHDFVEWLLRA;

### H-Aib-QGTFTSDYSKYLDS-K*-AAHDFVEWLLKA;

### H-Aib-QGTFTSDYSKYLDSKAA-K*-DFVAWLLSA;

### H-Aib-QGTFTSDYSKYLDSKAAHDFVAWLL-K*-A;

### H-Aib-QGTFTSDYSKYLD-K*-KAAHDFVAWLLRA;

### H-Aib-QGTFTSDYSRYLDS-K*-AAHDFVEWLLSA;

### H-Aib-QGTFTSDYSKYLDSKAAHDFV-K*-WLLSA;

### H-Aib-QGTFTSDYSLYLDS-K*-AAHDFVEWLLSA;

### H-Aib-QGTFTSDYSKYLDS-C*-AAHDFVEWLLSA;

### H-Aib-QGTFTSDYSKYLDSKAA-C*-DFVEWLLRA;

### H-Aib-QGTFTSDYSKYLD-S*-KAAHDFVEWLLSA;

### H-Aib-QGTFTSDYSKYLDK( )K*AAE( )DFVEWLLRA;

### H-Aib-QGTFTSDYSKYLDSK*AAHDFVE( )WLLK( )A;

### H-Aib-QGTFTSDYSKYLDSK*AAK( )DFVE( )WLLRA;

### H-Aib-QGTFTSDYSKYLDSK( )AAHE( )FVEWLLK*A; or

### H-Aib-QGTFTSDYSKYLDSK( )AAK*E( )FVEWLLRA,

where “*” indicates the position of a lipophilic substituent.

In any of the above combinations of compounds for use in methods of treatment, uses, and methods, Z¹ may include a hydrocarbon chain having 10 to 24 C atoms, 10 to 22 C atoms, or 10 to 20 C atoms (e.g., a dodecanoyl, 2-butyloctanoyl, tetradecanoyl, hexadecanoyl, heptadecanoyl, octadecanoyl, or eicosanoyl moiety) and/or Z² may be or may include one or more amino acid residues, for example, a γ-Glu, Glu, β-Ala or ε-Lys residue, or a 3-aminopropanoyl, 4-aminobutanoyl, 8-aminooctanoyl, or 8-amino-3,6-dioxaoctanoyl moiety (e.g., where the lipophilic substituent is selected from the group consisting of dodecanoyl-γ-Glu, hexadecanoly-γ-Glu, hexadecanoyl-Glu, hexadecanoyl-[3-aminopropanoyl], hexadecanoyl-[8-aminooctanoyl], hexadecanoyl-e-Lys, 2-butyloctanoyl-γ-Glu, octadecanoyl-γ-Glu, and hexadecanoyl-[4-aminobutanoyl]). In particular embodiments, Z has the formula:

### HSQGTFTSDYSKYLD-K(Hexadecanoyl-γ-Glu)-KAAHDFVEWLLRA;

### HSQGTFTSDYSKYLDSKAAHDFVEWL-K(Hexadecanoyl-γ-Glu)-RA;

### HSQGTFTSDYSKYLDSKAA-K(Hexadecanoyl-γ-Glu)-DFVEWLLRA;

### HSQGTFTSDYSKYLDSKAAHDFVEWLL-K(Hexadecanoyl-γ-Glu)-A;

### H-Aib-QGTFTSDYSKYLDS-K(Hexadecanoyl-γ-Glu)-AAHDFVEWLLRA;

### H-Aib-QGTFTSDYSKYLDS-K(Hexadecanoyl-γ-Glu)-AARDFVAWLLRA;

### H-Aib-QGTFTSDYSKYLDS-K(Hexadecanoyl-γ-Glu)-AAHDFVEWLLSA (Compound X);

### H-Aib-QGTFTSDYSKYLDSKAAHDFVEWLL-K(Hexadecanoyl-γ-Glu)-A;

### H-Aib-QGTFTSDYSKYLDS-K(Hexadecanoyl-γ-Glu)-AAHDFVEWLLKA;

### H-Aib-QGTFTSDYSKYLDS-K(Hexadecanoyl-γ-Glu)-AAHDFVE( )WLLK( )A; HSQGTFTSDYSKYLDS-K(Hexadecanoyl-γ-Glu)-AAHDFVEWLLRA; H-Aib-QGTFTSDYSKYLDSKAA-K(Hexadecanoyl-γ-Glu)-DFVAWLLRA;

### H-Aib-QGTFTSDYSKYLDS-K(Dodecanoyl-γ-Glu)-AAHDFVEWLLSA;

H-Aib-QGTFTSDYSKYLDS-K(Hexadecanoyl-[3-aminopropanoyl])-AAHDFVEWLLSA;
H-Aib-QGTFTSDYSKYLDS-K(Hexadecanoyl-[8-aminooctanoyl])-AAHDFVEWLLSA;

### H-Aib-QGTFTSDYSKYLDS-K(Hexadecanoyl-e-Lys)-AAHDFVEWLLSA;

### HSQGTFTSDYSKYLDS-K(Hexadecanoyl)-AAHDFVEWLLSA;

### HSQGTFTSDYSKYLDS-K(Octadecanoyl-γ-Glu)-AAHDFVEWLLSA;

### HSQGTFTSDYSKYLDS-K([2-Butyloctanoyl]-γ-Glu)-AAHDFVEWLLSA;

### HSQGTFTSDYSKYLDS-K(Hexadecanoyl-[4-Aminobutanoyl])-AAHDFVEWLLSA;

### HSQGTFTSDYSKYLDS-K(Octadecanoyl-γ-Glu)-AAHDFVEWLLSA;

### HSQGTFTSDYSKYLDS-K(Hexadecanoyl-E)-AAHDFVEWLLSA;

### H-Aib-QGTFTSDYSKYLDS-K(Hexadecanoyl)-AAHDFVEWLLSA;

### H-Aib-QGTFTSDYSKYLDS-K(Octadecanoyl-γ-Glu)-AAHDFVEWLLSA; H-Aib-QGTFTSDYSKYLDS-K([2-Butyloctanoyl]-γ-Glu)-AAHDFVEWLLSA;

### H-Aib-QGTFTSDYSKYLDS-K(Hexadecanoyl-[4-Aminobutanoyl])-AAHDFVEWLLSA;

### H-Aib-QGTFTSDYSKYLDS-K(Octadecanoyl-γ-Glu)-AAHDFVEWLLSA; or

### H-Aib-QGTFTSDYSKYLDS-K(Hexadecanoyl-E)-AAHDFVEWLLSA;

where residues marked “( )” participate in an intramolecular bond.

In other particular embodiments, Z has the formula:

### H-Aib-QGTFTSDYS-K(Hexadecanoyl-isoGlu)-YLDSKAAHDFVEWLLSA;

### H-Aib-QGTFTSDYSKYLD-K(Hexadecanoyl-isoGlu)-KAAHDFVEWLLSA;

### H-Aib-QGTFTSDYSKYLDSKAA-K(Hexadecanoyl-isoGlu)-DFVEWLLSA;

### H-Aib-QGTFTSDYSKYLDSKAAHDFV-K(Hexadecanoyl-isoGlu)-WLLSA;

### H-Aib-QGTFTSDYSKYLDS-K(Hexadecanoyl-isoLys)-AARDFVAWLLRA;

### H-Aib-QGTFTSDYSKYLDS-K(Hexadecanoyl-isoGlu)-AAKDFVEWLLSA;

### H-Aib-QGTFTSDYSKYLDE-K(Hexadecanoyl-isoGlu)-AAHDFVEWLLSA;

### H-Aib-QGTFTSDYSKYLDS-K(Hexadecanoyl-isoGlu)-AAHEFVEWLLSA;

### H-Aib-QGTFTSDYSKYLDS-K(Hexadecanoyl-isoGlu)-AAEDFVEWLLSA; or

### H-Aib-QGTFTSDYSKYLDS-K(Hexadecanoyl-isoGlu)-AAHDFVEWLLEA.

In another aspect, the invention features a combination of compounds for use in a method of treatment, a use, and a method for preventing or reducing weight gain; promoting weight loss; improving circulating glucose levels, glucose tolerance or circulating cholesterol levels; lowering circulating LDL levels; increasing HDL/LDL ratio; or treating a condition caused or characterized by excess body weight. The method includes administering to a mammalian (e.g., human) subject (e.g., having type 1 or type 2 diabetes) a combination of compounds including:

(a) a compound having the formula: R¹—Z—R², where Fe is H, C₁₋₄ alkyl, acetyl, formyl, benzoyl, or trifluoroacetyl; R² is OH or NH₂; and Z is a peptide having the formula V: His-Aib-Gln-Gly-Thr-Phe-Thr-Ser-Asp-Tyr-Ser-Lys-Tyr-Leu-Asp-Ser-X17-Ala-Ala-His-Asp-Phe-Val-Glu-Trp-Leu-Leu-X28; (V), where: X17 is X; X28 is Ser or absent; where X is selected from the group consisting of Glu, Lys, and Cys; and where the side chain of X is conjugated to a lipophilic substituent having the formula (I) Z¹, where Z¹ is a lipophilic moiety conjugated directly to the side chain of X; or (ii) Z¹Z², where Z¹ is a lipophilic moiety, Z² is a spacer, and Z¹ is conjugated to the side chain of X via Z²; and
(b) an insulin analogue (e.g., insulin glulisine (Apidra™), insulin lispro (Humalog™), Degludec, LY2963016, LY2605541, pegylated insulin Lispro, insulin glargine (Lantus™, Glaritus, Baselin, Baselog, Glarvia, BIOD-620), insulin detemir (Levemir™) Humulin, Huminsulin, insulin isophane (Humulin N, Insulatard, Novolin N), insulin and insulin isophane (Humulin 70/30, Humulin 50/50, Mixtard 30, Actraphane™ HM), insulin degludec and insulin aspart (DegludecPlus/NN-5401), insulin aspart (Novolog), insulin aspart and insulin protamine (Novolog mix, Novolog mix 70/30), insulin (NN-1953, IN-105, HinsBet, Capsulin, Nasulin, Afrezza, ORMD-0801, SuliXen, Humulin R), insulin buccal (Oral-lyn) and hyaluronidase insulin (Analog-PH20)). The combination of (a) and (b) may be administered in amounts that together are effective. The combination of (a) and (b) may be administered within one month (e.g., within three, two, or one weeks; six, five, four, three, two, or one days; or 18, 12, 8, 6, 4, 3, 2, or 1 hours) of each other. The condition caused or characterized by excess body weight may be selected from the group consisting of obesity, morbid obesity, obesity-linked inflammation, obesity-linked gallbladder disease, obesity-induced sleep apnea, metabolic syndrome, pre-diabetes, insulin resistance, glucose intolerance, type 2 diabetes, type I diabetes, hypertension, atherogenic dyslipidaemia, atherosclerosis, arteriosclerosis, coronary heart disease, peripheral artery disease, stroke, and microvascular disease. The combination of compounds for use in a method of treatment, a use, and a method may prevent or may reduce weight gain, may promote weight loss, and/or may improve circulating glucose levels. In certain embodiments, Z has the formula H-Aib-QGTFTSDYSKYLDS-K(Hexadecanoyl-isoGlu)-AAHDFVEWLLS or H-Aib-QGTFTSDYSKYLDS-K(Hexadecanoyl-isoGlu)-AAHDFVEWLL.

In another aspect, the invention features a combination of compounds for use in a method of treatment, a use, and a method for preventing or reducing weight gain; promoting weight loss; improving circulating glucose levels, glucose tolerance or circulating cholesterol levels; lowering circulating LDL levels; increasing HDL/LDL ratio; or treating a condition caused or characterized by excess body weight, the method including administering to a mammalian (e.g., human) subject (e.g., having type 1 or type 2 diabetes) a combination of compounds including (a) a compound having the formula: R¹—Z—R², where R¹ is H, C₁₋₄ alkyl, acetyl, formyl, benzoyl, or trifluoroacetyl; R² is OH or NH₂; and Z is a peptide having the formula VI: His-Aib-Glu-Gly-Thr-Phe-Thr-Ser-Asp-Tyr-Ser-Lys-Tyr-Leu-Asp-Ser-X17-Ala-Ala-His-Asp-Phe-Val-Glu-Trp-Leu-Leu-Ser-Ala; (VI) where X17 is X; where X is selected from the group consisting of Glu, Lys, and Cys; and where the side chain of X is conjugated to a lipophilic substituent having the formula: (i) Z¹, where Z¹ is a lipophilic moiety conjugated directly to the side chain of X; or (ii) Z¹Z², where Z¹ is a lipophilic moiety, Z² is a spacer, and Z¹ is conjugated to the side chain of X via Z²; and (b) an insulin analogue (e.g., insulin glulisine (Apidra™), insulin lispro (Humalog™), Degludec, LY2963016, LY2605541, pegylated insulin Lispro, insulin glargine (Lantus™, Glaritus, Basalin, Basalog, Glarvia, BIOD-620), insulin detemir (Levemir™) Humulin, Huminsulin, insulin isophane (Humulin N, Insulatard, Novolin N), insulin and insulin isophane (Humulin 70/30, Humulin 50/50, Mixtard 30, Actraphane™ HM), insulin degludec and insulin aspart (DegludecPlus/NN-5401), insulin aspart (Novolog), insulin aspart and insulin protamine (Novolog mix, Novolog mix 70/30), insulin (NN-1953, IN-105, HinsBet, Capsulin, Nasulin, Afrezza, ORMD-0801, SuliXen, Humulin R), insulin buccal (Oral-lyn) and hyaluronidase insulin (Analog-PH20)). The combination of (a) and (b) may be administered in amounts that together are effective. The combination of (a) and (b) may be administered within one month (e.g., within three, two, or one weeks; six, five, four, three, two, or one days; or 18, 12, 8, 6, 4, 3, 2, or 1 hours) of each other. The condition caused or characterized by excess body weight is selected from the group consisting of obesity, morbid obesity, obesity-linked inflammation, obesity-linked gallbladder disease, obesity-induced sleep apnea, metabolic syndrome, pre-diabetes, insulin resistance, glucose intolerance, type 2 diabetes, type I diabetes, hypertension, atherogenic dyslipidaemia, atherosclerosis, arteriosclerosis, coronary heart disease, peripheral artery disease, stroke, and microvascular disease. The combination of compounds for use in a method of treatment, a use, and a method may prevent or reduce weight gain, may promote weight loss, or may improve circulating glucose levels. In particular embodiments, Z has the formula: H-Aib-EGTFTSDYSKYLDS-K(Hexadecanoyl-isoGlu)-AAHDFVEWLLSA.

In a combination of compounds for use in a method of treatment, a use, and a method of the first aspect, the combination of (a) and (b) includes H—H-Aib-QGTFTSDYSKYLDS-K(Hexadecanoyl-isoGlu)-AAHDFVEWLLSA-NH₂ and insulin glargine; H—H-Aib-QGTFTSDYSKYLDS-K(Hexadecanoyl-isoGlu)-AAHDFVEWLLSA-NH₂ and insulin detemir; H—H-Aib-QGTFTSDYSKYLDS-K(Hexadecanoyl-isoGlu)-AAHDFVEWLLSA-NH₂ and glulisine (Apidra™); H—H-Aib-QGTFTSDYSKYLDS-K(Hexadecanoyl-isoGlu)-AAHDFVEWLLSA-NH₂ and insulin lispro (Humalog™); H—H-Aib-QGTFTSDYSKYLDS-K(Hexadecanoyl-isoGlu)-AAHDFVEWLLSA-NH₂ and degludec; H—H-Aib-QGTFTSDYSKYLDS-K(Hexadecanoyl-isoGlu)-AAHDFVEWLLSA-NH₂ and Actraphane HM; H—H-Aib-QGTFTSDYSKYLDS-K(Hexadecanoyl-isoGlu)-AAHDFVEWLLSA-NH₂ and LY2963016; H—H-Aib-QGTFTSDYSKYLDS-K(Hexadecanoyl-isoGlu)-AAHDFVEWLLSA-NH₂ and LY2605541; or H—H-Aib-QGTFTSDYSKYLDS-K(Hexadecanoyl-isoGlu)-AAHDFVEWLLSA-NH₂ and pegylated insulin Lispro.

In a particular embodiment, the combination of (a) and (b) includes H—H-Aib-QGTFTSDYSKYLDS- and insulin glargine, and the disease being treated is type 2 diabetes. In another particular embodiment, the combination of (a) and (b) includes H—H-Aib-QGTFTSDYSKYLDS-K(Hexadecanoyl-isoGlu)-AAHDFVEWLLSA-NH₂ and insulin detemir, and the disease being treated is type 2 diabetes. In another particular embodiment, the combination of (a) and (b) includes H—H-Aib-QGTFTSDYSKYLDS-K(Hexadecanoyl-isoGlu)-AAHDFVEWLLSA-NH₂ and glulisine (Apidra™), and the disease being treated is type 2 diabetes. In another particular embodiment, the combination of (a) and (b) includes H—H-Aib-QGTFTSDYSKYLDS-K(Hexadecanoyl-isoGlu)-AAHDFVEWLLSA-NH₂ and insulin lispro (Humelog™), and the disease being treated is type 2 diabetes. In another particular embodiment, the combination of (a) and (b) includes H—H-Aib-QGTFTSDYSKYLDS-K(Hexadecanoyl-isoGlu)-AAHDFVEWLLSA-NH₂ and degludec, and the disease being treated is type 2 diabetes. In a particular embodiment, the combination of (a) and (b) includes H—H-Aib-QGTFTSDYSKYLDS-K(Hexadecanoyl-isoGlu)-AAHDFVEWLLSA-NH₂ and Actraphane HM, and the disease being treated is type 2 diabetes. In another particular embodiment, the combination of (a) and (b) includes H—H-Aib-QGTFTSDYSKYLDS-K(Hexadecanoyl-isoGlu)-AAHDFVEWLLSA-NH₂ and LY2963016, and the disease being treated is type 2 diabetes. In another particular embodiment, the combination of (a) and (b) includes H—H-Aib-QGTFTSDYSKYLDS-K(Hexadecanoyl-isoGlu)-AAHDFVEWLLSA-NH₂ and LY2605541, and the disease being treated is type 2 diabetes. In another particular embodiment, the combination of (a) and (b) includes H—H-Aib-QGTFTSDYSKYLDS-K(Hexadecanoyl-isoGlu)-AAHDFVEWLLSA-NH₂ and pegylated insulin Lispro, and the disease being treated is type 2 diabetes.

In a particular embodiment, the combination of (a) and (b) includes H—H-Aib-QGTFTSDYSKYLDS-K(Hexadecanoyl-isoGlu)-AAHDFVEWLLSA-NH₂ and insulin glargine, and the administration results in weight loss (e.g., in an overweight or obese subject). In another particular embodiment, the combination of (a) and (b) includes H—H-Aib-QGTFTSDYSKYLDS-K(Hexadecanoyl-isoGlu)-AAHDFVEWLLSA-NH₂ and insulin detemir, and the administration results in weight loss (e.g., in an overweight or obese subject). In another particular embodiment, the combination of (a) and (b) includes H—H-Aib-QGTFTSDYSKYLDS-K(Hexadecanoyl-isoGlu)-AAHDFVEWLLSA-NH₂ and glulisine (Apidra™), and the administration results in weight loss (e.g., in an overweight or obese subject). In another particular embodiment, the combination of (a) and (b) includes H—H-Aib-QGTFTSDYSKYLDS-K(Hexadecanoyl-isoGlu)-AAHDFVEWLLSA-NH₂ and insulin lispro (Humalog™), and the administration results in weight loss (e.g., in an overweight or obese subject). In another particular embodiment, the combination of (a) and (b) includes H—H-Aib-QGTFTSDYSKYLDS-K(Hexadecanoyl-isoGlu)-AAHDFVEWLLSA-NH₂ and degludec, and the administration results in weight loss (e.g., in an overweight or obese subject). In another particular embodiment, the combination of (a) and (b) includes H—H-Aib-QGTFTSDYSKYLDS-K(Hexadecanoyl-isoGlu)-AAHDFVEWLLSA-NH₂ and Actraphane HM, and the administration results in weight loss (e.g., in an overweight or obese subject). In another particular embodiment, the combination of (a) and (b) includes H—H-Aib-QGTFTSDYSKYLDS-K(Hexadecanoyl-isoGlu)-AAHDFVEWLLSA-NH₂ and LY2963016, and the administration results in weight loss (e.g., in an overweight or obese subject). In another particular embodiment, the combination of (a) and (b) includes H—H-Aib-QGTFTSDYSKYLDS-K(Hexadecanoyl-isoGlu)-AAHDFVEWLLSA-NH₂ and LY2605541, and the administration results in weight loss (e.g., in an overweight or obese subject). In another particular embodiment, the combination of (a) and (b) includes H—H-Aib-QGTFTSDYSKYLDS-K(Hexadecanoyl-isoGlu)-AAHDFVEWLLSA-NH₂ and pegylated insulin Lispro, and the administration results in weight loss (e.g., in an overweight or obese subject).

In any of the above aspects, the combination of (a) and (b) are administered within one week, three days, two days, one day, 12 hours, or six hours of each other.

In a further aspect, the invention features a combination of compounds for use in a method of treatment, a use, and a method for preventing or reducing weight gain; promoting weight loss; improving circulating glucose levels, glucose tolerance or circulating cholesterol levels; lowering circulating LDL levels; increasing HDL/LDL ratio; or treating a condition caused or characterized by excess body weight in a mammalian subject (e.g., having type 1 or type 2 diabetes) that is receiving an insulin analogue (e.g., insulin glulisine (Apidra™), insulin lispro (Humalog™), Degludec, LY2963016, LY2605541, pegylated insulin Lispro, insulin glargine (Lantus™, Glaritus, Basalin, Basalog, Glarvia, BIOD-620), insulin detemir (Levemir™) Humulin, Huminsulin, insulin isophane (Humulin N, Insulatard, Novolin N), insulin and insulin isophane (Humulin 70/30, Humulin 50/50, Mixtard 30, Actraphane™ HM), insulin degludec and insulin aspart (DegludecPlus/NN-5401), insulin aspart (Novolog), insulin aspart and insulin protamine (Novolog mix, Novolog mix 70/30), insulin (NN-1953, IN-105, HinsBet, Capsulin, Nasulin, Afrezza, ORMD-0801, SuliXen, Humulin R), insulin buccal (Oral-lyn) and hyaluronidase insulin (Analog-PH20)), the method including administering to the subject a compound of the present invention in an effective amount. The condition caused or characterized by excess body weight may be selected from the group consisting of obesity, morbid obesity, obesity-linked inflammation, obesity-linked gallbladder disease, obesity-induced sleep apnea, metabolic syndrome, pre-diabetes, insulin resistance, glucose intolerance, type 2 diabetes, type I diabetes, hypertension, atherogenic dyslipidaemia, atherosclerosis, arteriosclerosis, coronary heart disease, peripheral artery disease, stroke, and microvascular disease. The combination of compounds for use in a method of treatment, a use, and a method may prevent or reduce weight gain, may promote weight loss, or may improve circulating glucose levels.

In any of the above aspects, the compound may be part of a composition including the compound, or a salt or derivative thereof, in admixture with a carrier. The composition may be a pharmaceutically acceptable composition, and the carrier may be a pharmaceutically acceptable carrier. The compound may be administered in a dosage of 0.1 nmol/kg body weight to 1 μmol/kg body weight (e.g., 3 nmol/kg to 30 nmol/kg). The insulin analogue may be administered in a dosage of 0.02 U/kg to 20 U/kg (e.g., 0.1 U/kg to 0.3 U/kg or about 0.2 U/kg). The compound may be administered every other week, weekly, every other day, daily, twice daily, or three times daily. The insulin analogue may be administered weekly, every other day, daily, twice daily, or three times daily.

The combination of compounds may be administered in an amount sufficient to reduce food intake in the subject by at least 5%, 10%, 15%, 20%, 25%, 30%, or 50%. The combination of compounds may be administered in an amount sufficient to reduce the subject's fasting blood glucose level by at least 1, 2, 3, 4, 5, 6, 8, 10, 11, 12, 15, or 20 mM. The combination of compounds may be administered in an amount sufficient to reduce the subject's HbA1c level by at least 0.1%, 0.2%, 0.3%, 0.4%, 0.5%, 0.6%, 0.8%, 1.0%, 1.5%, or 2.0%. The administration of the combination of compounds may result in a body weight reduction of at least 3%, 5%, 8%, 10%, 12%, 15% or 20% within 1 year of starting administration. The administration of the combination of compounds may result in a body weight reduction of at least 1%, 2%, 3%, 4%, 5%, 6%, 8%, 10% or 15% within six months of administration. The administration of the combination of compounds may result in a body weight reduction of at least 0.5%, 1%, 2%, 3%, 4%, 5%, 6%, 8%, 10% or 15% within three months of administration.

In any of the above aspects, the compound or insulin analogue may be administered subcutaneously, intravenously, intramuscularly, by inhalation, rectally, buccally, intraperitoneally, intraarticularly, or orally. The subject may be a human.

In another aspect, the invention features a kit including (a) a compound as recited in any of the above aspects; and (b) an insulin analogue (e.g., insulin glulisine (Apidra™), insulin lispro (Humalog™) Degludec, LY2963016, LY2605541, pegylated insulin Lispro, insulin glargine (Lantus™ Glaritus, Basalin, Basalog, Glarvia, BIOD-620), insulin detemir (Levemir™) Humulin, Huminsulin, insulin isophane (Humulin N, Insulatard, Novolin N), insulin and insulin isophane (Humulin 70/30, Humulin 50/50, Mixtard 30, Actraphane™ HM), insulin degludec and insulin aspart (DegludecPlus/NN-5401), insulin aspart (Novolog), insulin aspart and insulin protamine (Novolog mix, Novolog mix 70/30), insulin (NN-1953, IN-105, HinsBet, Capsulin, Nasulin, Afrezza, ORMD-0801, SuliXen, Humulin R), insulin buccal (Oral-lyn) and hyaluronidase insulin (Analog-PH20)), optionally including (c) instructions for administering (a) and (b) to a mammalian subject in need of preventing or reducing weight gain; promoting weight loss; improving circulating glucose levels, glucose tolerance or circulating cholesterol levels; lowering circulating LDL levels; increasing HDL/LDL ratio; or treatment for a condition caused or characterized by excess body weight.

Embodiments of the present invention will now be described by way of example and not limitation with reference to the accompanying figures. However, various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

### DESCRIPTION OF THE FIGURES

FIG. 1. Effect of treatment of 21 days s.c. administration of Lantus, Levemir, Compound X (H—H-Aib-QGTFTSDYSKYLDS-K(Hexadecanoyl-isoGlu)-AAHDFVEWLLSA-NH₂) and combinations thereof on body weight change (g). Data are averages +/−SEM with n=9-11. Data are compared by 2-way ANOVA vs. vehicle, ***p<0.001.

FIG. 2. Effect of 21 days s.c. administration of Lantus, Levemir, Compound X and combinations thereof on daily food intake and accumulated food intake and daily food intake. Data are averages +/−SEM with n=9-11. Data are compared by 2-way ANOVA vs. vehicle, ***p<0.001.

FIG. 3. Effect of 21 days s.c. administration of Lantus, Levemir, Compound X and combinations thereof on daily water intake and accumulated water intake. Data are averages +/−SEM with n=9-11. Data are compared by 2-way ANOVA vs. vehicle, ***p<0.001

FIG. 4. Effect of 21 days s.c. administration of Lantus, Levemir, Compound X and combinations thereof on delta-Blood Glucose (d-BG). Data are averages +/−SEM with n=9-11.

### DETAILED DESCRIPTION OF THE INVENTION

Throughout this specification, the conventional one letter and three letter codes for naturally occurring amino acids are used, as well as generally accepted three letter codes for other amino acids, including Aib (α-aminoisobutyric acid), Orn (ornithine), Dbu (2,4 diaminobutyric acid) and Dpr (2,3-diaminopropanoic acid).

Unless otherwise indicated, the L-isomeric forms of naturally occurring amino acids are referred to.

The term “native glucagon” refers to native human glucagon having the sequence H-His-Ser-Gln-Gly-Thr-Phe-Thr-Ser-Asp-Tyr-Ser-Lys-Tyr-Leu-Asp-Ser-Arg-Arg-Ala-Gln-Asp-Phe-Val-Gln-Trp-Leu-Met-Asn-Thr-OH.

Unless otherwise indicated, the L-isomeric forms of naturally occurring amino acids are referred to. The peptide sequence of a compound employed according to the invention differs from that of native glucagon at least at positions 18, 20, 24, 27, 28 and 29. In addition, it may differ from that of native glucagon at one or more of positions 12, 16 and 17.

Native glucagon has Arg at position 18. The compound employed in accordance with the invention has the small hydrophobic residue Ala at position 18 which is believed to increase potency at both glucagon and GLP-1 receptors but particularly the GLP-1 receptor.

The residues at positions 27, 28 and 29 of native glucagon appear to provide significant selectivity for the glucagon receptor. The substitutions at these positions with respect to the native glucagon sequence, particularly the Ala at position 29, may increase potency at and/or selectivity for the GLP-1 receptor, potentially without significant reduction of potency at the glucagon receptor. Further examples which may be included in the compounds to be employed in the invention include Leu at position 27 and Arg at position 28. Furthermore, Arg at position 28 may be particularly preferred when there is a Glu at position 24 with which it can form an intramolecular bridge, since this may increase its effect on potency at the GLP-1 receptor.

Substitution of the naturally occurring Met residue at position 27 (e.g., with Leu, Lys or Glu) also reduces the potential for oxidation, thereby increasing the chemical stability of the compounds.

Substitution of the naturally-occurring Asn residue at position 28 (e.g., by Arg or Ser) also reduces the potential for deamidation in acidic solution, thereby increasing the chemical stability of the compounds.

Potency and/or selectivity at the GLP-1 receptor, potentially without significant loss of potency at the glucagon receptor, may also be increased by introducing residues that are likely to stabilise an alpha-helical structure in the C-terminal portion of the peptide. It may be desirable, but is not believed essential, for this helical portion of the molecule to have an amphipathic character. Introduction of residues such as Leu at position 12 and/or Ala at position 24 may assist. Additionally or alternatively charged residues may be introduced at one or more of positions 16, 20, 24, and 28. Thus the residues of positions 24 and 28 may all be charged, the residues at positions 20, 24, and 28 may all be charged, or the residues at positions 16, 20, 24, and 28 may all be charged. For example, the residue at position 20 may be His or Arg, particularly His. The residue at position 24 may be Glu, Lys or Arg, particularly Glu. The residue at position 28 may be Arg. Introduction of an intramolecular bridge in this portion of the molecule, as discussed above, may also contribute to stabilising the helical character, e.g., between positions 24 and 28.

Substitution of one or both of the naturally-occurring Gln residues at positions 20 and 24 also reduces the potential for deamidation in acidic solution, so increasing the chemical stability of the compounds.

A substitution relative to the native glucagon sequence at position 12 (i.e., of Arg or Leu) may increase potency at both receptors and/or selectivity at the GLP-1 receptor.

C-terminal truncation of the peptide does not reduce potency of both receptors and/or selectivity of the GLP-1 receptor. In particular, truncation of position 29 or truncation of both position 28 and 29 does not reduce the receptor potency to any of the two receptors.

The side chain of one or more of the residues designated X (i.e., positions 16, 17, 20, 24, 27 and 28, and/or 30 if present) is conjugated to a lipophilic substituent. It will be appreciated that conjugation of the lipophilic substituent to a particular side chain may affect (e.g., reduce) certain of the benefits which the unconjugated side chain may provide at that position. The inventors have found that compounds of the invention provide a balance between the benefits of acylation and the benefits of particular substitutions relative to the native glucagon sequence.

Compositions employed in accordance with the invention may further be compounded in, or attached to, for example through covalent, hydrophobic and electrostatic interactions, a drug carrier, drug delivery system and advanced drug delivery system in order to further enhance stability of the compound, increase bioavailability, increase solubility, decrease adverse effects, achieve chronotherapy well known to those skilled in the art, and increase patient compliance or any combination thereof. Examples of carriers, drug delivery systems and advanced drug delivery systems include, but are not limited to, polymers, for example cellulose and derivatives, polysaccharides, for example dextran and derivatives, starch and derivatives, poly(vinyl alcohol), acrylate and methacrylate polymers, polylactic and polyglycolic acid and block co-polymers thereof, polyethylene glycols, carrier proteins, for example albumin, gels, for example, thermogelling systems, for example block co-polymeric systems well known to those skilled in the art, micelles, liposomes, microspheres, nanoparticulates, liquid crystals and dispersions thereof, L2 phase and dispersions there of, well known to those skilled in the art of phase behaviour in lipid-water systems, polymeric micelles, multiple emulsions, self-emulsifying, self-microemulsifying, cyclodextrins and derivatives thereof, and dendrimers.

Other groups have attempted to prolong the half life of GluGLP-1 dual agonist compounds by derivatisation with PEG (WO2008/101017). However such derivatisation appears to be most effective when applied to the C-terminus of the molecule rather than in the central core of the peptide backbone, and potency of these compounds is still decreased compared to the corresponding unmodified peptide.

By contrast, the compounds employed in the present invention retain high potency at both the glucagon and GLP-1 receptors while having significantly protracted pharmacokinetic profiles compared to the corresponding unmodified peptides.

Native glucagon has Ser at position 16. Substitution with Ala, Gly or Thr has been shown to reduce adenylate cyclase activation at the glucagon receptor significantly (Unson et al., Proc. Natl. Acad. Sci. 1994, 91, 454-458). Hence, derivatisation with a lipophilic substituent at position 16 would not have been expected to yield compounds retaining potency at the glucagon receptor, as is surprisingly shown by the compounds described in this specification. In WO2008/101017 a negatively charged residue was found to be desirable at position 16 to minimise loss of potency.

The presence of basic amino acids at positions 17 and 18 is generally believed to be necessary for full glucagon receptor activation (Unson et al., J. Biol. Chem. 1998, 273, 10308-10312). The present inventors have found that, when position 18 is alanine, substitution with a hydrophobic amino acid in position 17 can still yield a highly potent compound. Even compounds in which the amino acid in position 17 is derivatised with a lipophilic substituent retain almost full potency at both glucagon and GLP-1 receptors, as well as displaying a significantly protracted pharmacokinetic profile. This is so even when a lysine at position 17 is derivatised, converting the basic amine side chain into a neutral amide group.

The present inventors have also found that compounds with acylation at position 20 are still highly active dual agonists, despite indications from other studies that substitution in position 20 should be a basic amino acid having a side chain of 4-6 atoms in length to enhance GLP-1 receptor activity compared to glucagon (WO2008/101017). The compounds described herein retain both GLP-1 and glucagon receptor activity when position 20 is substituted with lysine and acylated.

### Peptide Synthesis

The peptide component of the compounds of the invention may be manufactured by standard solid or liquid phase synthetic methods, recombinant expression systems, or any other suitable method. Thus the peptides may be synthesized in a number of ways including for example, a method which comprises:

(a) synthesizing the peptide by means of solid phase or liquid phase methodology either stepwise or by fragment assembly, isolation and purification of the final peptide product;
(b) expressing a nucleic acid construct that encodes the peptide in a host cell and recovering the expression product from the host cell culture; or
(c) effecting cell-free in vitro expression of a nucleic acid construct that encodes the peptide and recovering the expression product;
or any combination of methods of (a), (b), and (c) to obtain fragments of the peptide, subsequently ligating the fragments to obtain the peptide, and recovering the peptide.

It may be preferred to synthesize the analogues of the invention by means of solid phase or liquid phase peptide synthesis. In this context, reference is given to WO 98/11125 and, amongst many others, Fields, G B et al., 2002, “Principles and practice of solid-phase peptide synthesis”. In: Synthetic Peptides (2nd Edition) and the examples herein.

### Lipophilic Substituent

One or more of the amino acid side chains in the compound employed in the invention is conjugated to a lipophilic substituent Z¹. Without wishing to be bound by theory, it is thought that the lipophilic substituent binds albumin in the blood stream, thus shielding the compounds of the invention from enzymatic degradation which can enhance the half-life of the compounds. It may also modulate the potency of the compound, e.g., with respect to the glucagon receptor and/or the GLP-1 receptor.

In certain embodiments, only one amino acid side chain is conjugated to a lipophilic substituent. In other embodiments, two amino acid side chains are each conjugated to a lipophilic substituent. In yet further embodiments, three or even more amino acid side chains are each conjugated to a lipophilic substituent. When a compound contains two or more lipophilic substituents, they may be the same or different.

The lipophilic substituent Z¹ may be covalently bonded to an atom in the amino acid side chain, or alternatively may be conjugated to the amino acid side chain by a spacer Z².

The term “conjugated” is used here to describe the physical attachment of one identifiable chemical moiety to another, and the structural relationship between such moieties. It should not be taken to imply any particular method of synthesis.

The spacer Z², when present, is used to provide a spacing between the compound and the lipophilic moiety.

The lipophilic substituent may be attached to the amino acid side chain or to the spacer via an ester, a sulphonyl ester, a thioester, an amide or a sulphonamide. Accordingly it will be understood that preferably the lipophilic substituent includes an acyl group, a sulphonyl group, an N atom, an O atom or an S atom which forms part of the ester, sulphonyl ester, thioester, amide or sulphonamide. Preferably, an acyl group in the lipophilic substituent forms part of an amide or ester with the amino acid side chain or the spacer.

The lipophilic substituent may include a hydrocarbon chain having 10 to 24 C atoms, e.g. 10 to 22 C atoms, e.g. 10 to 20 C atoms. Preferably it has at least 11 C atoms, and preferably it has 18 C atoms or fewer. For example, the hydrocarbon chain may contain 12, 13, 14, 15, 16, 17 or 18 carbon atoms. The hydrocarbon chain may be linear or branched and may be saturated or unsaturated. From the discussion above it will be understood that the hydrocarbon chain is preferably substituted with a moiety which forms part of the attachment to the amino acid side chain or the spacer, for example an acyl group, a sulphonyl group, an N atom, an O atom or an S atom. Most preferably the hydrocarbon chain is substituted with acyl, and accordingly the hydrocarbon chain may be part of an alkanoyl group, for example a dodecanoyl, 2-butyloctanoyl, tetradecanoyl, hexadecanoyl, heptadecanoyl, octadecanoyl or eicosanoyl group.

As mentioned above, the lipophilic substituent Z¹ may be conjugated to the amino acid side chain by a spacer Z². When present, the spacer is attached to the lipophilic substituent and to the amino acid side chain. The spacer may be attached to the lipophilic substituent and to the amino acid side chain independently by an ester, a sulphonyl ester, a thioester, an amide or a sulphonamide. Accordingly, it may include two moieties independently selected from acyl, sulphonyl, an N atom, an O atom or an S atom. The spacer may consist of a linear C₁₋₁₀ hydrocarbon chain or more preferably a linear C₁₋₅ hydrocarbon chain. Furthermore the spacer can be substituted with one or more substituents selected from C₁₋₆ alkyl, C₁₋₆ alkyl amine, C₁₋₆ alkyl hydroxy and C₁₋₆ alkyl carboxy.

The spacer may be, for example, a residue of any naturally occurring or unnatural amino acid. For example, the spacer may be a residue of Gly, Pro, Ala, Val, Leu, Ile, Met, Cys, Phe, Tyr, Trp, His, Lys, Arg, Gln, Asn, α-Glu, γ-Glu, ε-Lys, Asp, Ser, Thr, Gaba, Aib, β-Ala (i.e. 3-aminopropanoyl), 4-aminobutanoyl, 5-aminopentanoyl, 6-aminohexanoyl, 7-aminoheptanoyl, 8-aminooctanoyl, 9-aminononanoyl, 10-aminodecanoyl or 8-amino-3,6-dioxaoctanoyl. In certain embodiments, the spacer is a residue of Glu, γ-Glu, ε-Lys, β-Ala (i.e. 3-aminopropanoyl), 4-aminobutanoyl, 8-aminooctanoyl or 8-amino-3,6-dioxaoctanoyl. In the present invention, γ-Glu and isoGlu are used interchangeably.

The amino acid side chain to which the lipophilic substituent is conjugated is a side chain of a Glu, Lys, Ser, Cys, Dbu, Dpr or Orn residue. For example it may be a side chain of a Lys, Glu or Cys residue. Where two or more side chains carry a lipophilic substituent, they may be independently selected from these residues. Thus the amino acid side chain includes an carboxy, hydroxyl, thiol, amide or amine group, for forming an ester, a sulphonyl ester, a thioester, an amide or a sulphonamide with the spacer or lipophilic substituent.

An example of a lipophilic substituent comprising a lipophilic moiety Z¹ and spacer Z² is shown in the formula below:

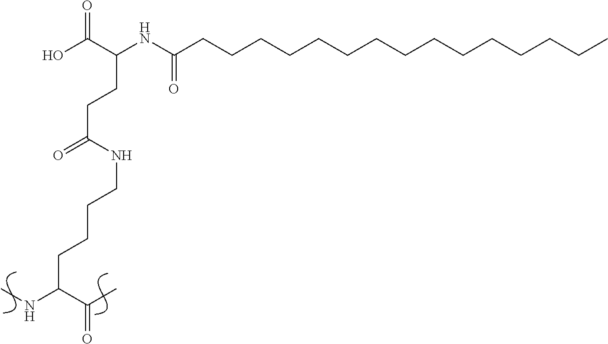 [SMILES: CCCCCCCCCCCCCCCC(=O)NC(CCC(=O)NCCCCC(NC)C(C)=O)C(=O)O]

Here, the side chain of a Lys residue from the peptide of formula I is covalently attached to an γ-Glu spacer (Z²) via an amide linkage. A hexadecanoyl group (Z¹) is covalently attached to the γ-Glu spacer via an amide linkage. This combination of lipophilic moiety and spacer, conjugated to a Lys residue, may be referred to by the short-hand notation K(Hexadecanoyl-γ-Glu), e.g., when shown in formulae of specific compounds. γ-Glu can also be referred to as isoGlu, and a hexadecanoyl group as a palmitoyl group. Thus it will be apparent that the notation (Hexadecanoyl-γ-Glu) is equivalent to the notations (isoGlu(Palm)) or (isoGlu(Palmitoyl)) as used for example in PCT/GB2008/004121.

The skilled person will be well aware of suitable techniques for preparing the compounds employed in the invention. For examples of suitable chemistry, see WO98/08871, WO00/55184, WO00/55119, Madsen et al (J. Med. Chem. 2007, 50, 6126-32), and Knudsen et al. 2000 (J. Med Chem. 43, 1664-1669). PEGylated and/or acylation have a short half-life (T½), which gives rise to burst increases of GluGLP-1 agonist concentrations. The glucagon receptor is thus being subjected to burst exposure to the glucagon agonism once (or twice) daily throughout the treatment period.

Without being bound to any theory repeated burst exposure of GluR to glucagon agonism seems to bring havoc to the lipid and free fatty acid trafficking between the liver and adipose tissue with the result that fat accumulates in the liver.

Constant exposure of GluR to glucagon agonism blocks accumulation of fat in the liver It has thus been found, that repeated treatment with glucagon or short acting dual GluGLP-1 agonists give rise to enlarged liver due to fat and glycogen accumulation (Chan et al., 1984, Exp. Mol. Path. 40, 320-327).

Repeated treatment with long-acting acylated dual GluGLP-1 agonists do not give rise to change in liver size (enlarged or shrunken) in normal weight subjects, but normalize liver lipid content (Day et al., 2009; Nat. Chem. Biol. 5, 749-57).

### Efficacy

Binding of the relevant compounds to GLP-1 or glucagon (Glu) receptors may be used as an indication of agonist activity, but in general it is preferred to use a biological assay which measures intracellular signalling caused by binding of the compound to the relevant receptor. For example, activation of the glucagon receptor by a glucagon agonist will stimulate cellular cyclic AMP (cAMP) formation. Similarly, activation of the GLP-1 receptor by a GLP-1 agonist will stimulate cellular cAMP formation. Thus, production of cAMP in suitable cells expressing one of these two receptors can be used to monitor the relevant receptor activity. Use of a suitable pair of cell types, each expressing one receptor but not the other, can hence be used to determine agonist activity towards both types of receptor.

The skilled person will be aware of suitable assay formats, and examples are provided below. The GLP-1 receptor and/or the glucagon receptor may have the sequence of the receptors as described in the examples. For example, the assays may make use the human glucagon receptor (Glucagon-R) having primary accession number GI: 4503947 (NP_{—}000151.1) and/or the human glucagon-like peptide 1 receptor (GLP-1R) having primary accession number GI:166795283 (NP_{—}002053.3). (Where sequences of precursor proteins are referred to, it should of course be understood that assays may make use of the mature protein, lacking the signal sequence).

EC₅₀ values may be used as a numerical measure of agonist potency at a given receptor. An EC₅₀ value is a measure of the concentration of a compound required to achieve half of that compound's maximal activity in a particular assay. Thus, for example, a compound having EC₅₀ [GLP-1R] lower than the EC₅₀ [GLP-1R] of native glucagon in a particular assay may be considered to have higher potency at the GLP-1R than glucagon.

The compounds described in this specification are typically Glu-GLP-1 dual agonists, i.e., they are capable of stimulating cAMP formation at both the glucagon receptor and the GLP-1R. The stimulation of each receptor can be measured in independent assays and afterwards compared to each other.

By comparing the EC₅₀ value for the glucagon receptor (EC₅₀ [Glucagon-R]) with the EC₅₀ value for the GLP-1 receptor (EC₅₀ [GLP-1R]) for a given compound the relative glucagon selectivity (%) of that compound can be found:

Relative Glucagon-R selectivity[Compound]=(1/EC₅₀[Glucagon-R])×100%/(1/EC₅₀[Glucagon-R]+1/EC₅₀[GLP-1R])

The relative GLP-1R selectivity can likewise be found:

Relative GLP-1R selectivity[Compound]=(1/EC₅₀[GLP-1R])×100%/(1/EC₅₀[Glucagon-R]+1/EC₅₀[GLP-1R])

A compound's relative selectivity allows its effect on the GLP-1 or glucagon receptor to be compared directly to its effect on the other receptor. For example, the higher a compound's relative GLP-1 selectivity is, the more effective that compound is on the GLP-1 receptor as compared to the glucagon receptor.

Using the assays described below, we have found the relative GLP-1 selectivity for human glucagon to be approximately 5%.

The compounds employed in the invention have a higher relative GLP-1R selectivity than human glucagon. Thus, for a particular level of glucagon-R agonist activity, the compound will display a higher level of GLP-1R agonist activity (i.e., greater potency at the GLP-1 receptor) than glucagon. It will be understood that the absolute potency of a particular compound at the glucagon and GLP-1 receptors may be higher, lower or approximately equal to that of native human glucagon, as long as the appropriate relative GLP-1R selectivity is achieved.

Nevertheless, the compounds employed in this invention may have a lower EC₅₀ [GLP-1R] than human glucagon. The compounds may have a lower EC₅₀ [GLP-1R] than glucagon while maintaining an EC₅₀ [Glucagon-R] that is less than 10-fold higher than that of human glucagon, less than 5-fold higher than that of human glucagon, or less than 2-fold higher than that of human glucagon.

It may be desirable that EC₅₀ of any given compound for both the Glucagon-R and GLP-1R should be less than 1 nM.

The compounds employed in the invention may have an EC₅₀ [Glucagon-R] that is less than two-fold that of human glucagon. The compounds may have an EC₅₀ [Glucagon-R] that is less than two-fold that of human glucagon and have an EC₅₀ [GLP-1R] that is less than half that of human glucagon, less than a fifth of that of human glucagon, or less than a tenth of that of human glucagon.

The relative GLP-1 selectivity of the compounds may be greater than 5% and less than 95%. For example, the compounds may have a relative selectivity of 5-20%, 10-30%, 20-50%, 30-70%, or 50-80%, or of 30-50%, 40-60%, 50-70% or 75-95%.

### Improving Circulating Glucose Levels, Glucose Tolerance or Circulating Cholesterol Levels

Normal blood sugar levels fluctuate depending on duration after last meal. A normal blood glucose level range for fasting individuals should be below 100 mg/dl and their level should be below 130-140 mg/dl or so around an hour after eating.

Ideally the fasting blood glucose levels should be around 90 mg/dl. Diabetes are diagnosed when fasting blood glucose levels are approaching 120 mg/dl or higher.

Blood sugar levels outside the normal range may be an indicator of a medical condition. A persistently high level is referred to as hyperglycemia; low levels are referred to as hypoglycemia. Diabetes mellitus is characterized by persistent hyperglycemia from any of several causes, and is the most prominent disease related to failure of blood sugar regulation. A temporarily elevated blood sugar level may also result from severe stress, such as trauma, stroke, myocardial infarction, surgery, or illness. Intake of alcohol causes an initial surge in blood sugar, and later tends to cause levels to fall. Also, certain drugs can increase or decrease glucose levels.

If blood sugar levels drop too low, a potentially fatal condition called hypoglycemia develops. Symptoms may include lethargy, impaired mental functioning; irritability; shaking, twitching, weakness in arm and leg muscles; pale complexion; sweating; paranoid or aggressive mentality and loss of consciousness. Brain damage is even possible.

If levels remain too high, appetite is suppressed over the short term. Long-term hyperglycemia causes many of the long-term health problems associated with diabetes, including eye, kidney, heart disease and nerve damage.

Type 1 diabetes is a lifelong condition that can be controlled with lifestyle adjustments and medical treatments. Keeping blood glucose levels under control can prevent or minimize complications. Insulin treatment is one component of a diabetes treatment plan for people with type 1 diabetes.

Insulin treatment replaces or supplements the body's own insulin, restoring normal or near-normal blood sugar levels. Many different types of insulin treatment can successfully control blood sugar levels; the best option depends upon a variety of individual factors. With a little extra planning, people with diabetes who take insulin can lead a full life and keep their blood sugar under control.

The central problem for those requiring external insulin is picking the right dose of insulin and the right timing.

Physiological regulation of blood glucose, as in the non-diabetic, would be best. Increased blood glucose levels after a meal is a stimulus for prompt release of insulin from the pancreas. The increased insulin level causes glucose absorption and storage in cells, reduces glycogen to glucose conversion, reducing blood glucose levels, and so reducing insulin release. The result is that the blood glucose level rises somewhat after eating, and within an hour or so, returns to the normal ‘fasting’ level. Even the best diabetic treatment with synthetic human insulin or even insulin analogs, however administered, falls far short of normal glucose control in the non-diabetic.

Complicating matters is that the composition of the food eaten affects intestinal absorption rates. Glucose from some foods is absorbed more (or less) rapidly than the same amount of glucose in other foods. In addition, fats and proteins cause delays in absorption of glucose from carbohydrates eaten at the same time.

It is a well known fact that insulin causes weight gain in patients with type 2 diabetes. Insulin is a hormone secreted by the pancreas in response to glucose intake usually in the diet. Its role is to drive glucose into the cells of the body where it is used as a source of energy (measured in calories). Insulin therefore pumps calories into cells. If this energy (glucose) is not used by the cells or is more than is needed, it is converted into an energy storage form known as fat. Because of these actions insulin is called an “anabolic” hormone.

The word “anabolic” means building up tissue. If a person is using his or her muscles and is physically active, the extra energy is converted into new (larger and/or stronger) muscles rather than fat. In a sense, a person who is sedentary, not using his muscles, getting more calories than he needs and taking insulin is in the midst of a “perfect (metabolic) storm” that will result in weight gain. The issue of insulin causing weight gain has long been a troubling aspect of the treatment for type 2 diabetes. It is not a problem in type 1 diabetes where patients have virtually no circulating insulin and need to receive it from an external source.

In type 2 diabetes the physiology is quite different. Here the body does make insulin, but the tissues are “resistant” to its effects. In fact, in the early stages of type 2 diabetes insulin levels can actually be high. This occurs because the tissues are resistant to insulin and higher insulin levels become necessary to drive sugar (glucose) into the cells and thereby drop the sugar level in the blood. The cause of insulin resistance is complex and is still a very active area of research. It appears that a certain type of fat tissue, fat that is contained in the abdomen (also called visceral adipose tissue), produces certain hormones and other substances that together cause insulin resistance. This was a major surprise in medicine when it was discovered only 10 or 15 years ago. Prior to that fat tissue was considered to be “metabolically inert”, which means that it was just a storage tissue and didn't affect metabolism. This was very far from the truth and visceral fat is now considered to be very active and complex metabolically. It produces a host of hormones (for example leptin, ghrelin and adiponectin) and other factors (cytokines) that have major influences on metabolism.

The discovery that insulin resistance was the central “lesion” in type 2 diabetes led to a whole area of research that resulted in linking type 2 diabetes to high blood pressure, truncal or abdominal obesity, abnormal blood lipids (elevated triglycerides and low HDL cholesterol) and high waist to hip ratio (the “apple” body type).

Using insulin to treat type 2 diabetes is problematic. The person with type 2 diabetes is usually overweight and circulating insulin levels may already be high. Adding additional insulin will certainly cause weight gain and this can actually make the insulin resistance worse. The usual justification is that using insulin will protect the remaining insulin-producing beta cells in the pancreas from having to work overtime. However, only a few months ago this issue was reviewed by one of the leading diabetes authorities in the world: Dr. Ralph DeFronzo, DeFronzo recently gave the prestigious Banting Lecture and it was published in the April 2009 issue of Diabetes. DeFronzo suggests that the American Diabetes Association guidelines for treatment of type 2 diabetes may be misguided and in need of revision.

Regarding insulin-induced weight gain, he notes that when insulin is added to the treatment regimen, “all of these insulin-based add-on studies have been associated with a high incidence of hypoglycemia [low blood sugar] and major weight gain (range 4.2-19.2 lbs, mean 8.5 lbs within 6-12 months or less) . . . . Moreover it is unclear why one would initiate insulin before exenatide [a newer non-insulin drug] since insulin rarely decreases A1C to <7% and is associated with significant weight gain . . . ” (Diabetes, Journal of the American Diabetes Association, April 2009, vol 58(4), page 786).Other potentially serious side-effects and related long term complications often associated with insulin treatment are well known. In particular, risk of developing hypoglycemia, allergy, resistance, and edema and related insulin side effects are well known short and longer-term side-effects of insulin treatment.

Glu-GLP-1 dual agonists of the present invention activates the GLP-1 receptor, a membrane-bound cell-surface receptor coupled to adenylyl cyclase by the stimulatory G-protein, Gs, in pancreatic beta cells. Glu-GLP-1 dual agonists of the present invention increases intracellular cyclic AMP (cAMP), leading to insulin release in the presence of elevated glucose concentrations. This insulin secretion subsides as blood glucose concentrations decrease and approach euglycemia. Glu-GLP-1 dual agonists of the present invention also decreases glucagon secretion in a glucose-dependent manner. The mechanism of blood glucose lowering also involves a delay in gastric emptying. GLP-1(7-37) has a half-life of 1.5-2 minutes due to degradation by the ubiquitous endogenous enzymes, dipeptidyl peptidase IV (DPP-IV) and neutral endopeptidases (NEP). Unlike native GLP-1, Glu-GLP-1 dual agonists of the present invention are stable against metabolic degradation by both peptidases and has a prolonged plasma half-life after subcutaneous administration. The pharmacokinetic profile of Glu-GLP-1 dual agonists of the present invention, which makes them suitable for once daily administration, is a result of self-association that delays absorption, plasma protein binding and stability against metabolic degradation by DPP-IV and NEP.

Combination of Glu-GLP-1 dual agonists of the present invention with insulin may have advantages over current type 2 diabetes therapies:
The combination acts in a glucose-dependent manner, meaning it will stimulate insulin secretion only when blood glucose levels are higher than normal. Consequently, it shows negligible risk of hypoglycemia.
The combination has the potential for inhibiting apoptosis and stimulating regeneration of beta cells (seen in animal studies).
The combination decreases appetite and maintains body weight, as shown in a head-to-head study versus glimepiride.
The combination lowers blood triglyceride
The combination has only mild and transient side effects, mainly gastrointestinal

For treatment of type 2 diabetes condition and in particular late stage type 2 diabetes condition, use of Glu-GLP-1 dual agonists in combination with insulin may further improve e.g. normalize circulating glucose levels, glucose tolerance or circulating cholesterol levels.

In one embodiment, the present invention is directed to treatment of diabetes melitus where a Glu-GLP-1 dual agonists of the present invention is co-administered with an insulin to improve the circulating glucose levels, glucose tolerance or circulating cholesterol levels.

In another embodiment, the present invention is directed to treatment of type 2 diabetes where a Glu-GLP-1 dual agonists of the present invention is co-administered with an insulin to improve the circulating glucose levels, glucose tolerance or circulating cholesterol levels.

### Insulin Analogues

The methods, kits, and compounds of the invention may use any insulin analogue known in the art.

Such insulin analogues comprise wild type insulin molecules, preferably of human genetic origin, as well as those which are modified chemically, e.g. by the exchange of single amino acids and/or the addition of side chains and/or the coupling with one or more medium sized molecules or polymers. Such insulin analogues also comprise compositions of such non-modified or modified insulins with other chemical substances which make them apt e.g. for the incorporation into specific medical compositions and/or mixtures with other insulin analogues.

In the context of this invention, human wildtype insulin is preferably produced recombinantly, which technique is per se known to the person skilled in the art. Such recombinant human insulins are also called Normal insulin. Products comprising recombinant human insulins are sold e.g. by the company Eli Lilly (Indianapolis, Ind., USA) under the product names Humulin™, Huminsulin™, Huminsulin™ basal, Humulin™ N, Humulin™ R, Humulin™ 70/30 and Humulin™ 50/50; or by the company Novo Nordisk (Bagsvrd, Denmark) under the product names Novolin™, Actrapid/Novolin™ and Actraphane™; or by the company Sanofi-Aventis (Schiltigheim, France) under the product names Insuman™ and Insuman™ basal.

This invention further pertains to genetically modified insulins. They are also preferably produced recombinantly. These modifications are intended to adapt the stability and/or absorption profile in the patient's body. An example for a genetically modified human insulin is Insulin aspart, which is characterized by the exchange of proline in position B28 against aspartic acid. It is marketed e.g. by Novo Nordisk, depending on further admixtures under the trade names NovoRapid™, Novolog™, Novolog™ mix, Novolog™ mix 70/30, NovoMix™ etc. Another example of a genetically modified insulin included herewith, is human insulin characterized by the two exchanges of (i) asparagine in position B3 against lysine and (ii) lysine in position B29 against glutamic acid. It was developed by Sanofi-Aventis and is sold e.g. under the trade name Apidra™ by this provider.

This invention further pertains to insulins modified or further modified by the covalent binding of chemical compounds. Such a modification leads to a specific absorption profile in the patient's body. One example is so-called Insulin detemir (Detemir) which is characterized by a fatty acid, esp. myristic acid, bound to the lysine amino acid at position B29 of human insulin. This specific myristylated insulin is marketed under the trade name Levemir™ by Novo Nordisk. Another example is Insulin Degludec™, developed by Novo Nordisk and described to be an ultralong-acting basal insulin. It is characterized by the deletion of the aminoacid alanin in position 330 and a carboxypentadecanoyl rest linked via a glutamic acid linker to position 29 of the same modified B-chain N^{6.B29}-[(N²-(15-carboxypentadecanoyl)-L-γ-glutamyl]-des-B30-L-threonine-insulin human; CAS no. 844439-96-9). Special preparations of it are sold under the names Degludec™ and DegludecPlus™ the latter being a combination product of Insulin Degludec™ and Insulin aspart.

Other chemical substances according to the invention to be mixed with insulins, comprise all chemical substances appropriate for the incorporation in medical compositions without being covalently bound to insulin. In the context of this invention, it is preferred that they interact with insulin and/or improve its intended physiological effect. Such chemical substances are per se known to the person skilled in the art. For example they comprise nuclear proteins like protamine or derivatives thereof, preferably Neutral Protamine Hagedorn (NPH). They can be used e.g. for the modification of the onset and/or the duration of the insulin action. Such insulines are e.g. marketed by Eli Lilly under the product names Insulin NPH or Insulin isophane or under the name NPH insulin by Novo Nordisk. Further examples are the above mentioned products Humulin™ N, Humulin™ R, Humulin™ 70/30 and Humulin™ 50/50.

Insulin Glargine (marketed by Sanofi-Aventis under the name Lantus™) is described below as the subject of one preferred mode of the invention. Alternatives and/or generic versions of this insulin, also included hereby, are e.g. the ones that are commercially available under the trade names Glaritus, Basalin and Basalog/Glarvia.

Further forms of insulins according to the invention can be characterized by their application route. For example they can be applied orally, nasaly or by inhalation. Examples are NN-1953, IN-105, Nasulin™ (developed by CPEX Pharmaceuticals; Wilmington, Del., USA), Afrezza, BIOD-620, Oral-lyn, HinsBet, Capsulin, Analog-PH20, ORMD-0801, SuliXen. Preferred are NN-1953, IN-105, BIOD-620 and Analog-PH20.

Examples of particular insulin analogues include insulin glulisine (Apidra™), glargine (Lantus™) Novorapid™, insulin lispro (Humalog™), Novomix™, Actraphane™ HM, insulin detemir (Levemir™) insulin glulisin (Apidra™), Degludec, LY2963016, LY2605541, and pegylated insulin Lispro, insulin glargine (Lantus™, Glaritus, Basalin, Basalog, Glarvia, BIOD-620), insulin detemir (Levemir™) Humulin, Huminsulin, insulin isophane (Humulin N, Insulatard, Novolin N), insulin and insulin isophane (Humulin 70/30, Humulin 50/50, Mixtard 30, Actraphane™ HM), insulin degludec and insulin aspart (DegludecPlus/NN-5401), insulin aspart (Novolog), insulin aspart and insulin protamine (Novolog mix, Novolog mix 70/30), insulin (NN-1953, IN-105, HinsBet, Capsulin, Nasulin, Afrezza, ORMD-0801, SuliXen, Humulin R), insulin buccal (Oral-lyn) and hyaluronidase insulin (Analog-PH20).

Further exemplary insulin analogues are described in detail below.

### Insulin Glargine (Lantus™)

Insulin glargine is an insulin analogue containing a substitution in the asparagine at position 21, along with the addition of two arginines to the carboxy terminal of the B chain. It is indicated for once-daily administration by injected subcutaneous injection and maintains a long duration of action and no pronounced peak concentration. Insulin glargine and related compounds and compositions are described in U.S. Pat. Nos. 5,656,722, 7,476,652, and 7,713,930. Exemplary compounds related to insulin glargine are described in U.S. Pat. No. 5,656,722 and have the sequence Asp^{A21}-Human insulin-Arg^{B31}-OH; Glu^{A21}-Human insulin-Arg^{B31}-OH; Gly^{A21}-Human insulin-Arg^{B31}—OH; Ser^{A21}-Human insulin-Arg^{B31}-OH; Thr^{A21}-Human insulin-Arg^{B31}-OH; Ala^{A21}-Human insulin-Arg^{B31}-OH; Asp^{A21}-Human insulin-Arg^{B31}-Arg^{B32}-OH; Glu^{A21}-Human insulin-Arg^{B31}-Arg^{B32}-OH; Gly^{A21}-Human insulin-Arg^{B31}-Arg^{B32}-OH; Ser^{A21}-Human insulin-Arg^{B31}-Arg^{B32}-OH; Thr^{A21}-Human insulin-Arg^{B31}-Arg^{B32}-OH; Ala^{A21}-Human insulin-Arg^{B31}-Arg^{B32}-OH; Asp^{A21}-Asn^{B10}-Human insulin-Arg^{B31}-OH; Glu^{A21}-Asn^{B10}-Human insulin-Arg^{B31}-OH; Gly^{A21}-Asn^{B10}-Human insulin-Arg^{B31}-OH; Ser^{A21}-Asn^{B10}-Human insulin-Arg^{B31}-OH; Thr^{A21}-Asn^{B10}-Human insulin-Arg^{B31}-OH; Ale^{A21}-Asn^{B10}-Human insulin-Arg^{B31}-OH; Asp^{A21}-Asn^{B10}-Human insulin-Arg^{B31}-Arg^{B32}-OH; Glu^{A21}-Asn^{B10}-Human insulin-Arg^{B31}-Arg^{B32}-OH; Gly^{A21}-Asn^{B10}-Human insulin-Arg^{B31}-Arg^{B32}-OH; Ser^{A21}-Asn^{B10}-Human insulin-Arg^{B31}-Arg^{B32}-OH; Thr^{A21}-Asn^{B10}-Human insulin-Arg^{B31}-Arg^{B32}-OH; and Ala^{A21}-Asn^{B10}-Human insulin-Arg^{B31}-Arg^{B32}-OH.

### Insulin Detemir (Levemir™)

Insulin detemir is a long-acting analogue of human insulin that has a C14 fatty acid chain (myristic acid) bound to the lysine at position B29 and the threonine at position 30 is omitted. Analogues of insulin detemir are described in U.S. Pat. Nos. 5,750,497; 5,866,538; 6,011,007; and 6,869,930, and have the formula

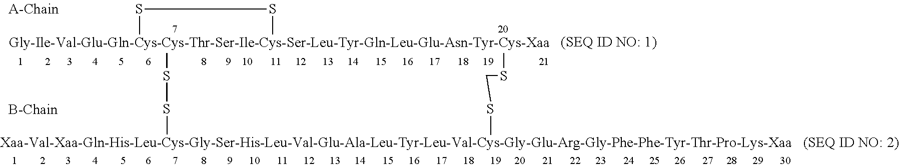

Xaa at positions A21 and B3 are, independently, any amino acid residue which can be coded for by the genetic code except Lys, Arg and Cys; Xaa at position B1 is Phe or is deleted; Xaa at position B30 is (a) a non-codable, lipophilic amino acid having from 10 to 24 carbon atoms, in which case an acyl group of a carboxylic acid with up to 5 carbon atoms is bound to the e-amino group of Lys^{B29}, (b) any amino acid residue which can be coded for by the genetic code except Lys, Arg and Cys, in which case the ε-amino group of Lys^{B29} has a lipophilic substituent or (c) deleted, in which case the ε-amino group of Lys^{B29} has a lipophilic substituent; and any Zn²⁺ complexes thereof, provided that when Xaa at position B30 is Thr or Ala, Xaa at positions A21 and B3 are both Asn, and Xaa at position B1 is Phe, then the insulin derivative is a Zn²⁺ complex.

In one preferred embodiment, the invention employs to a human insulin derivative in which the B30 amino acid residue is deleted or is any amino acid residue coded for by the genetic code except Lys, Arg, and Cys; the^{A21} and the^{B3} amino acid residues are, independently, any amino acid residues which can be coded for by the genetic code except Lys, Arg and Cys; Phe^{B1} may be deleted; the ε-amino group of Lys^{B29} has a lipophilic substituent which comprises at least 6 carbon atoms; and 2-4 Zn²⁺ ions may be bound to each insulin hexamer with the proviso that when B30 is Thr or Ala and A21 and B3 are both Asn, and Phe^{B1} is not deleted, then 2-4 Zn²⁺ ions are bound to each hexamer of the insulin derivative.

In another preferred embodiment, the invention employs to a human insulin derivative in which the B30 amino acid residue is deleted or is any amino acid residue which can be coded for by the genetic code except Lys, Arg and Cys; the A21 and the B3 amino acid residues are, independently, any amino acid residues which can be coded for by the genetic code except Lys, Arg and Cys, with the proviso that if the B30 amino acid residue is Ala or Thr, then at least one of the residues A21 and B3 is different from Asn; Phe^{B1} may be deleted; and the ε-amino group of Lys^{B29} has a lipophilic substituent which comprises at least 6 carbon atoms.

In another preferred embodiment, the invention employs to a human insulin derivative in which the B30 amino acid residue is deleted or is any amino acid residue which can be coded for by the genetic code except Lys, Arg and Cys; the A21 and the B3 amino acid residues are, independently, any amino acid residues which can be coded for by the genetic code except Lys, Arg and Cys; Phe may be deleted; the ε-amino group of Lys^{B29} has a lipophilic substituent which comprises at least 6 carbon atoms; and 2-4 Zn²⁺ ions are bound to each insulin hexamer.

In another embodiments, B30 amino acid residue is deleted, Asp, Glu, Thr, a lipophilic amino acid having at least 10 carbon atoms, a lipophilic α-amino acid having from 10 to 24 carbon atoms. In another preferred embodiment, the B30 amino acid is a straight chain, saturated, aliphatic α-amino acid having from 10 to 24 carbon atoms. In other preferred embodiments, the B30 amino acid is D- or L-N c dodecanoyllysine, α-amino decanoic acid, α-amino undecanoic acid, α-amino dodecanoic acid, α-amino tridecanoic acid, α-amino tetradecanoic acid, α-amino pentadecanoic acid, α-amino hexadecanoic acid, or an α-amino acid. In other preferred embodiments, the A21 amino acid residue is Ala, Gln, Gly, or Ser. In other preferred embodiments, the B3 amino acid residue is Asp, Gln, or Thr. In another preferred embodiment, the ε-amino group of Lys^{B29} has a lipophilic substituent which is an acyl group corresponding to a carboxylic acid having at least 6 carbon atoms. In another preferred embodiment, the ε-amino group of Lys^{B29} has a lipophilic substituent which is an acyl group, branched or unbranched, which corresponds to a carboxylic acid having a chain of carbon atoms 8 to 24 atoms long. In another preferred embodiment, the ε-amino group of Lys^{B29} has a lipophilic substituent which is an acyl group corresponding to a fatty acid having at least 6 carbon atoms. In another preferred embodiment, the ε-amino group of Lys^{B29} has a lipophilic substituent which is an acyl group corresponding to a linear, saturated carboxylic acid having from 6 to 24 carbon atoms. In another preferred embodiment, the ε-amino group of Lys^{B29} has a lipophilic substituent which is an acyl group corresponding to a linear, saturated carboxylic acid having from 8 to 12 carbon atoms. In another preferred embodiment, the ε-amino group of Lys^{B29} has a lipophilic substituent which is an acyl group corresponding to a linear, saturated carboxylic acid having from 10 to 16 carbon atoms. In another preferred embodiment, the ε-amino group of Lys^{B29} has a lipophilic substituent which is an oligo oxyethylene group comprising up to 10, preferably up to 5, oxyethylene units. In another preferred embodiment, the ε-amino group of Lys^{B29} has a lipophilic substituent which is an oligo oxypropylene group comprising up to 10, preferably up to 5, oxypropylene units. In other preferred embodiments, each insulin hexamer binds 2Zn²⁺ ions, 3Zn²⁺ ions, or 4Zn²⁺ ions.

Examples of preferred human insulin derivatives for use according to the present invention in which no Zn²⁺ ions are bound are the following: N^{εB29}-tridecanoyl des(B30) human insulin, N^{εB29}-tetradecanoyl des(B30) human insulin, N^{εB29}-decanoyl des(B30) human insulin, N^{εB29}-dodecanoyl des(B30) human insulin, N^{εB29}-tridecanoyl Gly^{A21} des(B30) human insulin, N^{εB29}-tetradecanoyl Gly^{A21} des(B30) human insulin, N^{εB29}-decanoyl Gly^{A21} des(B30) human insulin, N^{εB29}-dodecanoyl Gly^{A21} des(B30) human insulin, N^{εB29}-tridecanoyl Gly^{A21} Gln^{B3} des(B30) human insulin, N^{εB29}-tetradecanoyl Gly^{A21} Gln^{B3} des(B30) human insulin, N^{εB29}-decanoyl Gly^{A21} Gln^{B3} des(B30) human insulin, N^{εB29}-dodecanoyl Gly^{A21} Gln^{B3} des(B30) human insulin, N^{εB29}-tridecanoyl Ala^{A21} des(B30) human insulin, N^{εB29}-tetradecanoyl Ala^{A21} des(B30) human insulin, N^{εB29}-decanoyl Ala^{A21} des(B30) human insulin, N^{εB29}-dodecanoyl Ala^{A21} des(B30) human insulin, N^{εB29}-tridecanoyl Ala^{A21} Gln^{B3} des(B30) human insulin, N^{εB29}-tetradecanoyl Ala^{A21} Gln^{B3} des(B30) human insulin, N^{εB29}-decanoyl Ala^{A21} Gln^{B3} des(B30) human insulin, N^{εB29}-tridecanoyl Gln^{B3} des(B30) human insulin, N^{εB29}-dodecanoyl Ala^{A21} Gln^{B3} des(B30) human insulin, N^{εB29}-decanoyl Gln^{B3} des(B30) human insulin; N^{εB29}-tetradecanoyl Gln^{B3} des(B30) human insulin, N^{εB29}-dodecanoyl Gln^{B3} des(B30) human insulin, N^{εB29}-tridecanoyl Gly^{A21} human insulin, N^{εB29}-tetradecanoyl Gly^{A21} human insulin, N^{εB29}-decanoyl Gly^{A21} human insulin, N^{εB29}-dodecanoyl Gly^{A21} human insulin, N^{εB29}-tridecanoyl Gly^{A21} Gln^{B3} human insulin, N^{εB29}-tetradecanoyl Gly^{A21} Gln^{B3} human insulin, N^{εB29}-decanoyl Gly^{A21} Gln^{B3} human insulin, N^{εB29}-dodecanoyl Gly^{A21} Gln^{B3} human insulin, N^{εB29}-tridecanoyl Ala^{A21} human insulin, N^{εB29}-tridecanoyl Ala^{A21} human insulin, N^{εB29}-decanoyl Ala^{A21} human insulin, N^{εB29}-dodecanoyl Ala^{A21} human insulin, N^{εB29}-tridecanoyl Ala^{A21} Gln^{B3} human insulin, N^{εB29}-tetradecanoyl Ala^{A21} Gln^{B3} human insulin, N^{εB29}-decanoyl Ala^{A21} Gln^{B3} human insulin, N^{εB29}-dodecanoyl Ala^{A21} Gln^{B3} human insulin, N^{εB29}-tridecanoyl Gln^{B3} human insulin, N^{εB29}-tetradecanoyl Gln^{B3} human insulin, N^{εB29}-decanoyl Gln^{B3} human insulin, N^{εB29}-dodecanoyl Gln^{B3} human insulin, N^{εB29}-tridecanoyl Gln^{B30} human insulin, N^{εB29}-tetradecanoyl Gln^{B30} human insulin, N^{εB29}-decanoyl Gln^{B30} human insulin, N^{εB29}-dodecanoyl Gln^{B30} human insulin, N^{εB29}-tridecanoyl Gly^{A21} Glu^{B30} human insulin, N^{εB29}-tetradecanoyl Gly^{A21} Glu^{B30} human insulin, N^{εB29}-decanoyl Gly^{A21} Glu^{B30} human insulin, N^{εB29}-dodecanoyl Gly^{A21} Glu^{B30} human insulin, N^{εB29}-tridecanoyl Gly^{A21} Gln^{B3} Glu^{B30} human insulin, N^{εB29}-tetradecanoyl Gly^{A21} Gln^{B3} Glu^{B30} human insulin, N^{εB29}-decanoyl Gly^{A21} Gln^{B3} Glu^{B30} human insulin, N^{εB29}-dodecanoyl Gly^{A21} Gln^{B3} Glu^{B30} human insulin, N^{εB29}-tridecanoyl Ala^{A21} Glu^{B30} human insulin, N^{εB29}-tetradecanoyl Ala^{A21} Glu^{B30} human insulin, N^{εB29}-decanoyl Ala^{A21} Glu^{B30} human insulin, N^{εB29}-dodecanoyl Ala^{A21} Glu^{B30} human insulin, N^{εB29}-tridecanoyl Ala^{A21} Gln^{B3} Glu^{B30} human insulin, N^{εB29}-tetradecanoyl Ala^{A21} Gln^{B3} Glu^{B30} human insulin, N^{εB29}-decanoyl Ala^{A21} Gln^{B3} Glu^{B30} human insulin, N^{εB29}-dodecanoyl Ala^{A21} Gln^{B3} Glu^{B30} human insulin, N^{εB29}-tridecanoyl Gln^{B3} Glu^{B30} human insulin, N^{εB29}-tetradecanoyl Gln^{B3} Glu^{B30} human insulin, N^{εB29}-decanoyl Gln^{B3} Glu^{B30} human insulin, N^{εB29}-dodecanoyl Gln^{B3} Glu^{B30} human insulin.

Examples of preferred human insulin derivatives for use according to the present invention in which Zn²⁺ ions are bound per insulin hexamer are the following: (N^{εB29}-tridecanoyl des(B30) human insulin)₆, 2Zn²⁺, (N^{εB29}-tetradecanoyl des(B30) human insulin)₆, 2Zn²⁺, (N^{εB29}-decanoyl des(B30) human insulin)₆, 2Zn²⁺, (N^{εB29}-dodecanoyl des(B30) human insulin)₆, 2Zn²⁺, (N^{εB29}-tridecanoyl Gly^{A21} des(B30) human insulin)₆, 2Zn²⁺, (N^{εB29}-tetradecanoyl Gly^{A21} des(B30) human insulin)₆, 2Zn²⁺, (N^{εB29}-decanoyl Gly^{A21} des(B30) human insulin)₆, 2Zn²⁺, (N^{εB29}-dodecanoyl Gly^{A21} des(B30) human insulin)₆, 2Zn²⁺, (N^{εB29}-tridecanoyl Gly^{A21} Gln^{B3} des(B30) human insulin)₆, 2Zn²⁺, (N^{εB29}-tetradecanoyl Gly^{A21} Gln^{B3} des(B30) human insulin)₆, 2Zn²⁺, (N^{εB29}-decanoyl Gly^{A21} Gln^{B3} des(B30) human insulin)₆, 2Zn²⁺, (N^{εB29}-dodecanoyl Gly^{A21} Gln^{B3} des(B30) human insulin)₆, 2Zn²⁺, (N^{εB29}-tridecanoyl Ala^{A21} des(B30) human insulin)₆, 2Zn²⁺, (N^{εB29} tetradecanoyl Ala^{A21} des(B30) human insulin)₆, 2Zn²⁺, (N^{εB29}-decanoyl Ala^{A21} des(B30) human insulin)₆, 2Zn²⁺, (N^{εB29}-dodecanoyl Ala^{A21} des(B30) human insulin)₆, 2Zn²⁺, (N^{εB29}-tridecanoyl Ala^{A21} Gln^{B3} des(B30) human insulin)₆, 2Zn²⁺, (N^{εB29}-tetradecanoyl Ala^{A21} Gln^{B3} des(B30) human insulin)₆, 2Zn²⁺, (N^{εB29}-decanoyl Ala^{A21} Gln^{B3} des(B30) human insulin)₆, 2Zn²⁺, (N^{εB29}-dodecanoyl Ala^{A21} Gln^{B3} des(B30) human insulin)₆, 2Zn²⁺, (N^{εB29}-tridecanoyl Gln^{B3} des(B30) human insulin)₆, 2Zn²⁺, (N^{εB29}-tetradecanoyl Gln^{B3} des(B30) human insulin)₆, 2Zn²⁺, (N^{εB29}-decanoyl Gln^{B3} des(B30) human insulin)₆, 2Zn²⁺, (N^{εB29}-dodecanoyl Gln^{B3} des(B30) human insulin)₆, 2Zn²⁺, (N^{εB29}-tridecanoyl human insulin)₆, 2Zn²⁺, (N^{εB29}-tetradecanoyl human insulin)₆, 2Zn²⁺, (N^{εB29}-decanoyl human insulin)₆, 2Zn²⁺, (N^{εB29}-dodecanoyl human insulin)₆, 2Zn²⁺, (N^{εB29}-tridecanoyl Gly^{A21} human insulin)₆, 2Zn²⁺, (N^{εB29}-tetradecanoyl Gly^{A21} human insulin)₆, 2Zn²⁺, (N^{εB29}-decanoyl Gly^{A21} human insulin)₆, 2Zn²⁺, (N^{εB29}-dodecanoyl Gly^{A21} human insulin)₆, 2Zn²⁺, (N^{εB29}-tridecanoyl Gly^{A21} Gln^{B3} human insulin)₆, 2Zn²⁺, (N^{εB29}-tetradecanoyl Gly^{A21} Gln^{B3} human insulin)₆, 2Zn²⁺, (N^{εB29}-decanoyl Gly^{A21} Gln^{B3} human insulin)₆, 2Zn²⁺, (N^{εB29}-dodecanoyl Gly^{A21} Gln^{B3} human insulin)₆, 2Zn²⁺, (N^{εB29}-tridecanoyl Ala^{A21} human insulin)₆, 2Zn²⁺, (N^{εB29}-tetradecanoyl Ala^{A21} human insulin)₆, 2Zn²⁺, (N^{εB29}-decanoyl Ala^{A21} human insulin)₆, 2Zn²⁺, (N^{εB29}-dodecanoyl Ala^{A21} human insulin)₆, 2Zn²⁺, (N^{εB29}-tridecanoyl Ala^{A21} Gln^{B3} human insulin)₆, 2Zn²⁺, (N^{εB29}-tetradecanoyl Ala^{A21} Gln^{B3} human insulin)₆, 2Zn²⁺, (N^{εB29}-decanoyl Ala^{A21} Gln^{B3} human insulin)₆, 2Zn²⁺, (N^{εB29}-dodecanoyl Ala^{A21} Gln^{B3} human insulin)₆, 2Zn²⁺, (N^{εB29}-tridecanoyl Gln^{B3} human insulin)₆, 2Zn²⁺, (N^{εB29}-tetradecanoyl Gln^{B3} human insulin)₆, 2Zn²⁺, (N^{εB29}-decanoyl Gln^{B3} human insulin)₆, 2Zn²⁺, (N^{εB29}-dodecanoyl Gln^{B3} human insulin)₆, 2Zn²⁺, (N^{εB29}-tridecanoyl Gln^{B30} human insulin)₆, 2Zn²⁺, (N^{εB29}-tetradecanoyl Glu^{B30} human insulin)₆, 2Zn²⁺, (N^{εB29}-decanoyl Glu^{B30} human insulin)₆, 2Zn²⁺, (N^{εB29}-dodecanoyl Glu^{B30} human insulin)₆, 2Zn²⁺, (N^{εB29}-tridecanoyl Gly^{A21} Glu^{B30} human insulin)₆, 2Zn²⁺, (N^{εB29}-tetradecanoyl Gly^{A21} Glu^{B39} human insulin)₆, 2Zn²⁺, (N^{εB29}-decanoyl Gly^{A21} Glu^{B30} human insulin)₆, 2Zn²⁺, (N^{εB29}-dodecanoyl Gly^{A21} Glu^{B30} human insulin)₆, 2Zn²⁺, (N^{εB29}-tridecanoyl Gly^{A21} Gln^{B3} Glu^{B30} human insulin)₆, 2Zn²⁺, (N^{εB29}-tetradecanoyl Gly^{A21} Gln^{B3} Glu^{B30} human insulin)₆, 2Zn²⁺, (N^{εB29}-decanoyl Gly^{A21} Gln^{B3} Glu^{B30} human insulin)₆, 2Zn²⁺, (N^{εB29}-dodecanoyl Gly^{A21} Gln^{B3} Glu^{B30} human insulin)₆, 2Zn²⁺, (N^{εB29}-tridecanoyl Ala^{A21} Glu^{B30} human insulin)₆, 2Zn²⁺, (N^{εB29}-tetradecanoyl Ala^{A21} Glu^{B30} human insulin)₆, 2Zn²⁺, (N^{εB29}-decanoyl Ala^{A21} Glu^{B30} human insulin)₆, 2Zn²⁺, (N^{εB29}-dodecanoyl Ala^{A21} Glu^{B30} human insulin)₆, 2Zn²⁺, (N^{εB29}-tridecanoyl Ala^{A21} Gln^{B3} Glu^{B30} human insulin)₆, 2Zn²⁺, (N^{εB29}-tetradecanoyl Ala^{A21} Gln^{B3} Glu^{B30} human insulin)₆, 2Zn²⁺, (N^{εB29}-decanoyl Ala^{A21} Gln^{B3} Glu^{B30} human insulin)₆, 2Zn²⁺, (N^{εB29}-dodecanoyl Ala^{A21} Gln^{B3} Glu^{B30} human insulin)₆, 2Zn²⁺, (N^{εB29}-tridecanoyl Gln^{B3} Glu^{B30} human insulin)₆, 2Zn²⁺, (N^{εB29}-tetradecanoyl Gln^{B3} Glu^{B30} human insulin)₆, 2Zn²⁺, (N^{εB29}-decanoyl Gln^{B3} Glu^{B30} human insulin)₆, 2Zn²⁺, (N^{εB29}-dodecanoyl Gln^{B3} Glu^{B30} human insulin)₆, 2Zn²⁺.

Examples of preferred human insulin derivatives for use according to the present invention in which three Zn²⁺ ions are bound per insulin hexamer are the following: (N^{εB29}-tridecanoyl des(B30) human insulin)₆, 3Zn²⁺, (N^{εB29}-tetradecanoyl des(B30) human insulin)₆, 3Zn²⁺, (N^{εB29}-decanoyl des(B30) human insulin)₆, 3Zn²⁺, (N^{εB29}-dodecanoyl des(B30) human insulin)₆, 3Zn²⁺, (N^{εB29}-tridecanoyl Gly^{A21} des(B30) human insulin)₆, 3Zn²⁺, (N^{εB29}-tetradecanoyl Gly^{A21} des(B30) human insulin)₆, 3Zn²⁺, (N^{εB29}-decanoyl Gly^{A21} des(B30) human insulin)₆, 3Zn²⁺, (N^{εB29}-dodecanoyl Gly^{A21} des(B30) human insulin)₆, 3Zn²⁺, (N^{εB29}-tridecanoyl Gly^{A21} Gln^{B3} des(B30) human insulin)₆, 3Zn²⁺, (N^{εB29}-tetradecanoyl Gly^{A21} Gln^{B3} des(B30) human insulin)₆, 3Zn²⁺, (N^{εB29}-decanoyl Gly^{A21} Gln^{B3} des(B30) human insulin)₆, 3Zn²⁺, (N^{εB29}-dodecanoyl Gly^{A21} Gln^{B3} des(B30) human insulin)₆, 3Zn²⁺, (N^{εB29}-tridecanoyl Ala^{A21} des(B30) human insulin)₆, 3Zn²⁺, (N^{εB29}-tetradecanoyl Ala^{A21} des(B30) human insulin)₆, 3Zn²⁺, (N^{εB29}-decanoyl Ala^{A21} des(B30) human insulin)₆, 3Zn²⁺, (N^{εB29}-dodecanoyl Ala^{A21} des(B30) human insulin)₆, 3Zn²⁺, (N^{εB29}-tridecanoyl Ala^{A21} Gln^{B3} des(B30) human insulin)₆, 3Zn²⁺, (N^{εB29}-tetradecanoyl Ala^{A21} des(B30) human insulin)₆, 3Zn²⁺, (N^{εB29}-decanoyl Ala^{A21} Gln^{B3} des(B30) human insulin)₆, 3Zn²⁺, (N^{εB29}-dodecanoyl Ala^{A21} Gln^{B3} des(B30) human insulin)₆, 3Zn²⁺, (N^{εB29}-tridecanoyl Gln^{B3} des(B30) human insulin)₆, 3Zn²⁺, (N^{εB29}-tetradecanoyl Gln^{B3} des(B30) human insulin)₆, 3Zn²⁺, (N^{εB29}-decanoyl Gln^{B3} des(B30) human insulin)₆, 3Zn²⁺, (N^{εB29}-dodecanoyl Gln^{B3} des(B30) human insulin)₆, 3Zn²⁺, (N^{εB29}-tridecanoyl human insulin)₆, 3Zn²⁺, (N^{εB29}-tetradecanoyl human insulin)₆, 3Zn²⁺, (N^{εB29}-decanoyl human insulin)₆, 3Zn²⁺, (N^{εB29}-dodecanoyl human insulin)₆, 3Zn²⁺, (N^{εB29}-tridecanoyl Gly^{A21} human insulin)₆, 3Zn²⁺, (N^{εB29}-tetradecanoyl Gly^{A21} human insulin)₆, 3Zn²⁺, (N^{εB29}-decanoyl Gly^{A21} human insulin)₆, 3Zn²⁺, (N^{εB29}-dodecanoyl Gly^{A21} human insulin)₆, 3Zn²⁺, (N^{εB29}-tridecanoyl Gly^{A21} Gln^{B3} human insulin)₆, 3Zn²⁺, (N^{εB29}-tetradecanoyl Gly^{B3} human insulin)₆, 3Zn²⁺, (N^{εB29}-decanoyl Gly^{A21} Gln^{B3} human insulin)₆, 3Zn²⁺, (N^{εB29}-dodecanoyl Gly^{A21} Gln^{B3} human insulin)₆, 3Zn²⁺, (N^{εB29}-tridecanoyl Ala^{A21} human insulin)₆, 3Zn²⁺, (N^{εB29}-tetradecanoyl Ala^{A21} human insulin)₆, 3Zn²⁺, (N^{εB29}-decanoyl Ala^{A21} human insulin)₆, 3Zn²⁺, (N^{εB29}-dodecanoyl Ala^{A21} human insulin)₆, 3Zn²⁺, (N^{εB29}-tridecanoyl Ala^{A21} Gln^{B3} human insulin)₆, 3Zn²⁺, (N^{εB29}-tetradecanoyl Ala^{A21} Gln^{B3} human insulin)₆, 3Zn²⁺, (N^{εB29}-decanoyl Ala^{A21} Gln^{B3} human insulin)₆, 3Zn²⁺, (N^{εB29}-dodecanoyl Ala^{A21} Gln^{B3} human insulin)₆, 3Zn²⁺, (N^{εB29}-tridecanoyl Gln^{B3} human insulin)₆, 3Zn²⁺, (N^{εB29}-tetradecanoyl Gln^{B3} human insulin)₆, 3Zn²⁺, (N^{εB29}-decanoyl Gln^{B3} human insulin)₆, 3Zn²⁺, (N^{εB29}-dodecanoyl Gln^{B3} human insulin)₆, 3Zn²⁺, (N^{εB29}-tridecanoyl Glu^{B30} human insulin)₆, 3Zn²⁺, (N^{εB29}-tetradecanoyl Glu^{B30} human insulin)₆, 3Zn²⁺, (N^{εB29}-decanoyl Glu^{B30} human insulin)₆, 3Zn²⁺, (N^{εB29}-dodecanoyl Glu^{B30} human insulin)₆, 3Zn²⁺, (N^{εB29}-tridecanoyl Gly^{A21} Glu^{B30} human insulin)₆, 3Zn²⁺, (N^{εB29}-tetradecanoyl Gly^{A21} Glu^{B30} human insulin)₆, 3Zn²⁺, (N^{εB29}-decanoyl Gly^{A21} Glu^{B30} human insulin)₆, 3Zn²⁺, (N^{εB29}-dodecanoyl Gly^{A21} Glu^{B30} human insulin)₆, 3Zn²⁺, (N^{εB29}-tridecanoyl Gly^{A21} Gln^{B3} Glu^{B30} human insulin)₆, 3Zn²⁺, (N^{εB29}-tetradecanoyl Gly^{A21} Glu^{B30} human insulin)₆, 3Zn²⁺, (N^{εB29}-decanoyl Gly^{A21} Gln^{B3} Glu^{B30} human insulin)₆, 3Zn²⁺, (N^{εB29}-dodecanoyl Gly^{A21} Gln^{B3} Glu^{B30} human insulin)₆, 3Zn²⁺, (N^{εB29}-tridecanoyl Ala^{A21} Glu^{B30} human insulin)₆, 3Zn²⁺, (N^{εB29}-tetradecanoyl Ala^{A21} Glu^{B30} human insulin)₆, 3Zn²⁺, (N^{εB29}-decanoyl Ala^{A21} Glu^{B30} human insulin)₆, 3Zn²⁺, (N^{εB29}-dodecanoyl Ala^{A21} Glu^{B30} human insulin)₆, 3Zn²⁺, (N^{εB29}-tridecanoyl Ala^{B21} Glu^{B30} human insulin)₆, 3Zn²⁺, (N^{εB29}-tetradecanoyl Ala^{A21} Gln^{B3} Glu^{B30} human insulin)₆, 3Zn²⁺, (N^{εB29}-decanoyl Ala^{A21} Gln^{B3} Glu^{B30} human insulin)₆, 3Zn²⁺, (N^{εB29}-dodecanoyl Ala^{A21} Gln^{B3} Glu^{B30} human insulin)₆, 3Zn²⁺, (N^{εB29}-tridecanoyl Gln^{B3} Glu^{B30} human insulin)₆, 3Zn²⁺, (N^{εB29}-tetradecanoyl Gln^{B3} Glu^{B30} human insulin)₆, 3Zn²⁺, (N^{εB29}-decanoyl Gln^{B3} Glu^{B30} human insulin)₆, 3Zn²⁺, (N^{εB29}-dodecanoyl Gln^{B3} Glu^{B30} human insulin)₆, 3Zn²⁺.

Examples of preferred human insulin derivatives for use according to the present invention in which four Zn²⁺ ions are bound per insulin hexamer are the following: (N^{εB29}-tridecanoyl des(B30) human insulin)₆, 4Zn²⁺, (N^{εB29}-tetradecanoyl des(B30) human insulin)₆, 4Zn²⁺, (N^{εB29}-decanoyl des(B30) human insulin)₆, 4Zn²⁺, (N^{εB29}-dodecanoyl des(B30) human insulin)₆, 4Zn²⁺, (N^{εB29}-tridecanoyl Gly^{A21} des(B30) human insulin)₆, 4Zn²⁺, (N^{εB29}-tetradecanoyl Gly^{A21} des(B30) human insulin)₆, 4Zn²⁺, (N^{εB29}-decanoyl Gly^{A21} des(B30) human insulin)₆, 4Zn²⁺, (N^{εB29}-dodecanoyl Gly^{A21} des(B30) human insulin)₆, 4Zn²⁺, (N^{εB29}-tridecanoyl Gly^{A21} Gln^{B3} des(B30) human insulin)₆, 4Zn²⁺, (N^{εB29}-tetradecanoyl Gly^{A21} Gln^{B3} des(B30) human insulin)₆, 4Zn²⁺, (N^{εB29}-decanoyl Gly^{A21} Gln^{B3} des(B30) human insulin)₆, 4Zn²⁺, (N^{εB29}-dodecanoyl Gly^{A21} Gln^{B3} des(B30) human insulin)₆, 4Zn²⁺, (N^{εB29}-tridecanoyl Ala^{A21} des(B30) human insulin)₆, 4Zn²⁺, (N^{εB29}-tetradecanoyl Ala^{A21} des(B30) human insulin)₆, (N^{εB29}-decanoyl Ala^{A21} des(B30) human insulin)₆, 4Zn²⁺, (N^{εB29}-dodecanoyl Ala^{A21} des(B30) human insulin)₆, 4Zn²⁺, (N^{εB29}-tridecanoyl Ala^{A21} Gln^{B3} des(B30) human insulin)₆, 4Zn²⁺, (N^{εB29}-tetradecanoyl Ala^{A21} Gln^{B3} des(B30) human insulin)₆, 4Zn²⁺, (N^{εB29}-decanoyl Ala^{A21} Gln^{B3} des(B30) human insulin)₆, 4Zn²⁺, (N^{εB29}-dodecanoyl Ala^{A21} Gln^{B3} des(B30) human insulin)₆, 4Zn²⁺, (N^{εB29}-tridecanoyl Gln^{B3} des(B30) human insulin)₆, 4Zn²⁺, (N^{εB29}-tetradecanoyl Gln^{B3} des(B30) human insulin)₆, 4Zn²⁺, (N^{εB29}-decanoyl Gln^{B3} des(B30) human insulin)₆, 4Zn²⁺, (N^{εB29}-dodecanoyl Gln^{B3} des(B30) human insulin)₆, 4Zn²⁺, (N^{εB29}-tridecanoyl human insulin)₆, 4Zn²⁺, (N^{εB29}-tetradecanoyl human insulin)₆, 4Zn²⁺, (N^{εB29}-decanoyl human insulin)₆, 4Zn²⁺, (N^{εB29}-dodecanoyl human insulin)₆, 4Zn²⁺, (N^{εB29}-tridecanoyl Gly^{A21} human insulin)₆, 4Zn²⁺, (N^{εB29}-tetradecanoyl Gly^{A21} human insulin)₆, 4Zn²⁺, (N^{εB29}-decanoyl Gly^{A21} human insulin)₆, 4Zn²⁺, (N^{εB29}-dodecanoyl Gly^{A21} human insulin)₆, 4Zn²⁺, (N^{εB29}-tridecanoyl Gly^{A21} Gln^{B3} human insulin)₆, 4Zn²⁺, (N^{εB29}-tetradecanoyl Gly^{A21} Gln^{B3} human insulin)₆, 4Zn²⁺, (N^{εB29}-decanoyl Gly^{A21} Gln^{B3} human insulin)₆, 4Zn²⁺, (N^{εB29}-dodecanoyl Gly^{A21} Gln^{B3} human insulin)₆, 4Zn²⁺, (N^{εB29}-tridecanoyl Ala^{A21} human insulin)₆, 4Zn²⁺, (N^{εB29}-tetradecanoyl Ala^{A21} human insulin)₆, 4Zn²⁺, (N^{εB29}-decanoyl Ala^{A21} human insulin)₆, 4Zn²⁺, (N^{εB29}-dodecanoyl Ala^{A21} human insulin)₆, 4Zn²⁺, (N^{εB29}-tridecanoyl Ala^{A21} Gln^{B3} human insulin)₆, 4Zn²⁺, (N^{εB29}-tetradecanoyl Ala^{A21} Gln^{B3} human insulin)₆, 4Zn²⁺, (N^{εB29}-decanoyl Ala^{A21} Gln^{B3} human insulin)₆, 4Zn²⁺, (N^{εB29}-dodecanoyl Ala^{A21} Gln^{B3} human insulin)₆, 4Zn²⁺, (N^{εB29}-tridecanoyl Gln^{B3} human insulin)₆, 4Zn²⁺, (N^{εB29}-tetradecanoyl Gln^{B3} human insulin)₆, 4Zn²⁺, (N^{εB29}-decanoyl Gln^{B3} human insulin)₆, 4Zn²⁺, (N^{εB29}-dodecanoyl Gln^{B3} human insulin)₆, 4Zn²⁺, (N^{εB29}-trideca noyl Glu^{B30} human insulin)₆, 4Zn²⁺, (N^{εB29}-tetradecanoyl Glu^{B30} human insulin)₆, 4Zn²⁺, (N^{εB29}-decanoyl Glu^{B30} human insulin)₆, 4Zn²⁺, (N^{εB29}-dodecanoyl Glu^{B30} human insulin)₆, 4Zn²⁺, (N^{εB29}-tridecanoyl Gly^{A21} Glu^{B30} human insulin)₆, 4Zn²⁺, (N^{εB29}-tetradecanoyl Gly^{A21} Glu^{B30} human insulin)₆, 4Zn²⁺, (N^{εB29}-decanoyl Gly^{A21} Glu^{B30} human insulin)₆, 4Zn²⁺, (N^{εB29}-dodecanoyl Gly^{A21} Glu^{B30} human insulin)₆, 4Zn²⁺, (N^{εB29}-tridecanoyl Gly^{A21} Gln^{B3} Glu^{B30} human insulin)₆, 4Zn²⁺, (N^{εB29}-tetradecanoyl Gly^{A21} Gln^{B3} Glu^{B30} human insulin)₆, 4Zn²⁺, (N^{εB29}-decanoyl Gly^{A21} Gln^{B3} Glu^{B30} human insulin)₆, 4Zn²⁺, (N^{εB29}-dodecanoyl Gly^{A21} Gln^{B3} Glu^{B30} human insulin)₆, 4Zn²⁺, (N^{εB29}-tridecanoyl Ala^{A21} Glu^{B30} human insulin)₆, 4Zn²⁺, (N^{εB29}-tetradecanoyl Ala^{A21} Glu^{B30} human insulin)₆, 4Zn²⁺, (N^{εB29}-decanoyl Ala^{A21} Glu^{B30} human insulin)₆, 4Zn²⁺, (N^{εB29}-dodecanoyl Ala^{A21} Glu^{B30} human insulin)₆, 4Zn²⁺, (N^{εB29}-tridecanoyl Ala^{A21} Gln^{B3} Glu^{B30} human insulin)₆, 4Zn²⁺, (N^{εB29}-tetradecanoyl Ala^{A21} Gln^{B3} Glu^{B30} human insulin)₆, 4Zn²⁺, (N^{εB29}-decanoyl Ala^{A21} Gln^{B3} Glu^{B30} human insulin)₆, 4Zn²⁺, (N^{εB29}-dodecanoyl Ala^{A21} Gln^{B3} Glu³⁰ human insulin)₆, 4Zn²⁺, (N^{εB29}-tridecanoyl Gln^{B3} Glu^{B30} human insulin)₆, 4Zn²⁺, (N^{εB29}-tetradecanoyl Gln^{B3} Glu^{B30} human insulin)₆, 4Zn²⁺, (N^{εB29}-decanoyl Gln^{B3} Glu^{B30} human insulin)₆, 4Zn²⁺, (N^{εB29}-dodecanoyl Gln^{B3} Glu^{B30} human insulin)₆, 4Zn²⁺,

### Insulin Glulisine (Apidran™)

Insulin glulisine is a human insulin analogue in which the asparagine at position B3 is replaced by lysine and the lysine in position B29 is replaced by glutamic acid (3^{B}-lysine 29^{B}-glutamic acid-human insulin). Analogues of insulting glulisine are described in U.S. Pat. No. 6,221,633 and have the formula:

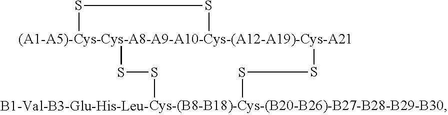

where (A1-A5) are the amino acid residues in the positions A1 to A5 of the A chain of human insulin or animal insulin, (A12-A19) are the amino acid residues in the positions A12 to A19 of the A chain of human insulin or animal insulin, (B8-B18) are the amino acid residues in the positions B8 to B18 of the B chain of human insulin or animal insulin, (B20-B26) are the amino acid residues in the positions B20 to B26 of the B chain of human insulin or animal insulin, A8, A9, A10 are the amino acid residues in the positions A8, A9 and A10 of the A chain of human insulin or animal insulin, A21 is Asn, Asp, Gly, Ser, Thr or Ala, B30 is —OH or the amino acid residue in position B30 of the B chain of human insulin or animal insulin, B1 is Phe or a hydrogen atom, B3 is a naturally occurring basic amino acid residue, B27, B28 and B29 are the amino acid residues in the positions B27, B28 and B29 of the B chain of human insulin or animal insulin or in each case are another naturally occurring amino acid residue, where at least one of the amino acid residues in the positions B27, B28 and B29 of the B chain is replaced by another naturally occurring amino acid residue.

Of the twenty naturally occurring amino acids which are genetically encodable, the amino acids Gly, Ala, Val, Leu, Ile, Ser, Thr, Cys, Met, Asn, Gln, Phe, Tyr, Trp and Pro are designated here as neutral amino acids, the amino acids Arg, Lys and His are designated as basic amino acids and the amino acids Asp and Glu are designated as acidic amino acids.

Preferably, the insulin derivative or its physiologically tolerable salt for use according to the present invention is a derivative of bovine insulin, porcine insulin or human insulin, namely an insulin derivative or a physiologically tolerable salt thereof of the formula 1, which is distinguished in that A8 is (Ala), A9 is Ser, A10 is Val and B30 is Ala (amino acid residues A8 to A10 and B30 of bovine insulin), A8 is Thr, A9 is Ser and A10 is Ile (amino acid residues A8 to A10 of the insulins of man or pig), where B30 is Ala (amino acid residue B30 of porcine insulin) or B30 is Thr (amino acid residue B30 of human insulin). Particularly preferably, an insulin derivative or a physiologically tolerable salt thereof of the formula I with the amino acid residues A8 to A10 and B30 of human insulin is furthermore distinguished in that (A1-A5) are the amino acid residues in the positions A1 to A5 of the A chain of human insulin, (A12-A19) are the amino acid residues in the positions A12 to A19 of the A chain of human insulin, (B8-B18) are the amino acid residues in the positions B8 to B18 of the B chain of human insulin, and (B20-B26) are the amino acid residues in the positions B20 to B26 of the B chain of human insulin. Further preferred embodiments of the present invention are an insulin derivative or a physiologically tolerable salt thereof of the formula 1, wherein the amino acid residue in position B1 of the B chain is Phe or an insulin derivative or a physiologically tolerable salt thereof of the formula 1, wherein the amino acid residue in position B3 of the B chain is a His, Lys or Arg.

Further preferred embodiments for use in the present invention are an insulin derivative or a physiologically tolerable salt thereof of the formula 1, wherein at least one of the amino acid residues in the positions B27, B28 and B29 of the B chain is replaced by a naturally occurring amino acid residue which is selected from the group consisting of the neutral or of the acidic amino acids, an insulin derivative or a physiologically tolerable salt thereof of the formula I, wherein at least one of the amino acid residues in the positions B27, B28 and B29 of the B chain is a naturally occurring amino acid residue which is selected from the group consisting of Ile, Asp and Glu, preferably wherein at least one of the amino acid residues in the positions B27, B28 of the B chain is replaced by a naturally occurring amino acid residue which is selected from the group consisting of the neutral amino acids, or particularly preferably wherein at least one of the amino acid residues in the positions B27, 628 and B29 of the B chain is Ile, or an insulin derivative or a physiologically tolerable salt thereof of the formula I, wherein at least one of the amino acid residues in the positions B27, 628 and B29 of the B chain is a naturally occurring amino acid residue which is selected from the group consisting of the acidic amino acids, preferably wherein at least one of the amino acid residues in the positions B27, B28 and B29 of the B chain is Asp, preferably wherein the amino acid residue in position B27 or B28 of the B chain is Asp, or wherein at least one of the amino acid residues in the positions B27, B28 and B29 of the B chain is Glu.

A preferred embodiment for use in the present invention is also an insulin derivative or a physiologically tolerable salt thereof of the formula I, wherein the amino acid residue in position B29 of the B chain is Asp. Further preferred embodiments are an insulin derivative or a physiologically tolerable salt thereof of the formula I, wherein the amino acid residue in position B27 of the B chain is Glu, an insulin derivative or a physiologically tolerable salt thereof of the formula I, wherein the amino acid residue in position B28 of the B chain is Glu, or an insulin derivative or a physiologically tolerable salt thereof of the formula I, wherein the amino acid residue in position B29 of the B chain is Glu.

Very particularly preferably, an insulin derivative or a physiologically tolerable salt thereof is one which is distinguished in that the B chain has the sequence Phe Val Lys Gln His Leu Cys Gly Ser His Leu Val Glu Ala Leu Tyr Leu Val Cys Gly Glu Arg Gly Phe Phe Tyr Thr Pro Glu Thr, for example Lys(B3), Glu(B29)-human insulin, or an insulin derivative or a physiologically tolerable salt thereof which is distinguished in that the amino acid residue in position B27 of the B chain is Ile, preferably an insulin derivative or a physiologically tolerable salt thereof which is distinguished in that the B chain has the sequence Phe Val Lys Gln His Leu Cys Gly Ser His Leu Val Glu Ala Leu Tyr Leu Val Cys Gly Glu Arg Gly Phe Phe Tyr Ile Pro Lys Thr, for example Lys (B3), Ile (B27)-human insulin, or an insulin derivative or a physiologically tolerable salt thereof of the formula I, wherein the amino acid residue in position B28 of the B chain is Ile, preferably an insulin derivative or a physiologically tolerable salt thereof which is distinguished in that the B chain has the sequence Phe Val Lys Gln His Leu Cys Gly Ser His Leu Val Glu Ala Leu Tyr Leu Val Cys Gly Glu Arg Gly Phe Phe Tyr Thr Ile Lys Thr, for example Lys (B3), Ile (B28)-human insulin.

Particularly preferably, an insulin derivative or a physiologically tolerable salt thereof is distinguished in that the amino acid residue in position B28 of the B chain is Ile and the amino acid residue in position A21 is Asp, is preferably one wherein the A chain has the sequence Gly Ile Val Glu Gln Cys Cys Thr Ser Ile Cys Ser Leu Tyr Gln Leu Tyr Gln Leu Glu Asn Tyr Cys Asp and the B chain has the sequence Phe Val Lys Gln His Leu Cys Gly Ser His Leu Val Glu Ala Leu Tyr Leu Val Cys Gly Glu Arg Gly Phe Phe Pyr Thr Ile Lys Thr (Lys(B3), Ile(B28), Asp(A21)-human insulin).

### Insulin Lispro and Pegylated Forms Thereof.

Insulin Lispro is a fast acting insulin analogue having in which the penultimate lysine and proline residues on the C-terminal end of the B-chain are reversed (Lys^{B28} Pro^{B29} human insulin). This compound is described in U.S. Pat. No. 5,461,031.

Pegylated lispro is described, for example, in PCT Publication WO/2009/152128 and have the formula P-[(A)-(B)], or a pharmaceutically acceptable salt thereof, where A is the A-chain of insulin lispro; B is the B-chain of insulin lispro; and P is a PEG having a molecular weight in the range from about 20 kDa to about 40 kDa, and wherein the A and B are properly cross-linked and P is attached either directly or indirectly via a covalent bond to the alpha-amino group of the glycine at position 1 of A, the alpha-amino group of the phenylalanine at position 1 of B, or the epsilon-amino group of the lysine at position 28 of B. The present invention may also employ compositions comprising a plurality of mono- and di-PEGylated insulin lispro compounds wherein greater than about 75% of the PEGylated insulin lispro compounds in the composition are mono-PEGylated compounds of the formula. The present invention may also employ compositions comprising mono-PEGylated insulin compounds of the formula wherein greater than about 50% of the mono-PEGylated compounds in the composition have a PEG covalently attached either directly or indirectly to the epsilon-amino group of the lysine at position 28 of the B-chain.

### Degludec

Degludec is a human insulin analogue having the formula:

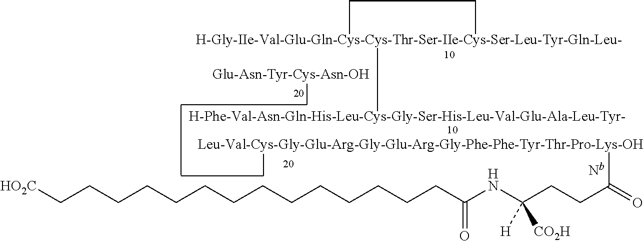

Degludec is indicated for thrice weekly injection and has a long half life. Also included is DegludecPlus (NN-5401).

### Actraphane™

Actraphane is a range of insulin suspensions for injection. These include Actraphane 10 (soluble insulin 10% and isophane insulin 90%), Actraphane 20 (soluble insulin 20% and isophane insulin 80%), Actraphane 30 (soluble insulin 30% and isophane insulin 70%); Actraphane 40 (soluble insulin 40% and isophane insulin 60%), and Actraphane 50 (soluble insulin 50% and isophane insulin 50%).

### LY2963016

LY2963016, a new insulin glargin analogue, is described, for example, in the PCT publications WO 2004096854, WO 2003053460, WO 2003053339, WO 2010080609, WO 2010080606, WO 2010014946, WO 2010002283, WO 2009132129, WO 2009129250, WO 2007081824, the US publication No. 20100099601, the Chinese publication CN 101519446, or the Australian Publication No. AU 2008326324.

### LY2605541

LY2605541, a new insulin analogue, is described, for example, in the PCT publications WO 2004096854, WO 2003053460, WO 2003053339, WO 2010080609, WO 2010080606, WO 2010014946, WO 2010002283, WO 2009132129, WO 2009129250, WO 2007081824, the US publication No. 20100099601, the Chinese patent No. CN 101519446, or the Australian publication AU 2008326324.

### Additional Insulin Analogues and Derivatives

New insulin derivatives with an extremely delayed time effect profile for use in the treatment of diabetes are described, for example, in the PCT publications WO 2009087081, WO 2009087082 and the German publications DE 102008003568 and DE 102008003566.

These analogues have a B chain modified with a terminal amidated basic amino acid (arginine or lysine), an N-terminal arginine or lysine on the A-chain, position 8 of the A-chain substituted (A8) with histidine and position 21 of the A (A21) chain substituted with a glycine. Acidic amino acids at positions A5, A15, A18, B-1, B0, and B1-B4 are also substituted. The prolonged time-action profile allows these variants to be used without the risk of inducing hypoglycemia.

Also, the isoelectric point of the insulin is changed by addition or substitution of negative and positive charged amino acid residues and by an amidation of the C-terminal carboxy group of the B chain and histidine in position 8 of the insulin A chain. The prolonged time-action profile allows these variants to be used without the risk of inducing hypoglycemia.

### Further Forms of Insulin

Insulins applied orally, nasaly or by inhalation includes but is not limited to NN-1953, IN-105, Nasulin, Afrezza, BIOD-620, Oral-lyn, HinsBet, Capsulin, Analog-PH20, ORMD-0801 and SuliXen. In a preferred embodiment is included NN-1953, IN-105, BIOD-620 and Analog-PH20.

### Therapeutic Uses

The methods, kits, and compounds of the invention may provide an attractive treatment option for metabolic diseases including obesity and diabetes mellitus (diabetes).

Diabetes comprises a group of metabolic diseases characterized by hyperglycemia resulting from defects in insulin secretion, insulin action, or both. Acute signs of diabetes include excessive urine production, resulting compensatory thirst and increased fluid intake, blurred vision, unexplained weight loss, lethargy, and changes in energy metabolism. The chronic hyperglycemia of diabetes is associated with long-term damage, dysfunction, and failure of various organs, notably the eyes, kidneys, nerves, heart and blood vessels. Diabetes is classified into type 1 diabetes, type 2 diabetes and gestational diabetes on the basis on pathogenetic characteristics.

Type 1 diabetes accounts for 5-10% of all diabetes cases and is caused by auto-immune destruction of insulin-secreting pancreatic β-cells.

Type 2 diabetes accounts for 90-95% of diabetes cases and is a result of a complex set of metabolic disorders. Type 2 diabetes is the consequence of endogenous insulin production becoming insufficient to maintain plasma glucose levels below the diagnostic thresholds.

Gestational diabetes refers to any degree of glucose intolerance identified during pregnancy.

Pre-diabetes includes impaired fasting glucose and impaired glucose tolerance and refers to those states that occur when blood glucose levels are elevated but below the levels that are established for the clinical diagnosis for diabetes.

A large proportion of people with type 2 diabetes and pre-diabetes are at increased risk of morbidity and mortality due to the high prevalence of additional metabolic risk factors including abdominal obesity (excessive fat tissue around the abdominal internal organs), atherogenic dyslipidemia (blood fat disorders including high triglycerides, low HDL cholesterol and/or high LDL cholesterol, which foster plaque buildup in artery walls), elevated blood pressure (hypertension) a prothrombotic state (e.g., high fibrinogen or plasminogen activator inhibitor-1 in the blood), hypertriglyceridemia, hypercholesterolemia and proinflammatory state (e.g., elevated C-reactive protein in the blood).

Conversely, obesity confers an increased risk of developing pre-diabetes, type 2 diabetes as well as, e.g., certain types of cancer, obstructive sleep apnea and gall-blader disease.

Dyslipidaemia is associated with increased risk of cardiovascular diasese. High Density Lipoprotein (HDL) is of clinical importance since an inverse correlation exists between plasma HDL concentrations and risk of atherosclerotic disease. The majority of cholesterol stored in atherosclerotic plaques originates from LDL and hence elevated concentrations Low Density Lipoproteins (LDL) is closely associated with atherosclerosis. The HDL/LDL ratio is a clinical risk indictor for atherosclerosis and coronary atherosclerosis in particular.

Without wishing to be bound by any particular theory, it is believed that the compounds employed in the invention act as GluGLP-1 dual agonists. The dual agonist may combine the effect of glucagon, e.g., on fat metabolism with the effect of GLP-1, e.g., on blood glucose levels and food intake. They might therefore act to accelerate elimination of excessive adipose tissue, induce sustainable weight loss, and improve glycaemic control. Dual GluGLP-1 agonists might also act to reduce cardiovascular risk factors such as high cholesterol and LDL-cholesterol. Dual GluGLP-1 agonists might also act to reduce circulating triacylglycerol levels and lowering circulating free fatty acids.

The compounds employed in the present invention can therefore be used as pharmaceutical agents for preventing weight gain, promoting weight loss, reducing excess body weight or treating obesity (e.g., by control of appetite, feeding, food intake, calorie intake, and/or energy expenditure), including morbid obesity, as well as associated diseases and health conditions including but not limited to obesity linked inflammation, obesity linked gallbladder disease and obesity induced sleep apnea. The compounds employed in the invention may also be used for treatment of insulin resistance, glucose intolerance, pre-diabetes, increased fasting glucose, type 2 diabetes, hypertension, dyslipidemia (or a combination of these metabolic risk factors), atherosclerois, arteriosclerosis, coronary heart disease, peripheral artery disease and stroke. These are all conditions which can be associated with obesity. However, the effects of the compounds of the invention on these conditions may be mediated in whole or in part via an effect on body weight, or may be independent thereof.

### Pharmaceutical Compositions

The compounds employed in the present invention, or salts thereof, may be formulated as pharmaceutical compositions prepared for storage or administration, which typically comprise a therapeutically effective amount of a compound of the invention, or a salt thereof, in a pharmaceutically acceptable carrier.

The therapeutically effective amount of a compound employed in the present invention will depend on the route of administration, the type of mammal being treated, and the physical characteristics of the specific mammal under consideration. These factors and their relationship to determining this amount are well known to skilled practitioners in the medical arts. This amount and the method of administration can be tailored to achieve optimal efficacy, and may depend on such factors as weight, diet, concurrent medication and other factors, well known to those skilled in the medical arts. The dosage sizes and dosing regimen most appropriate for human use may be guided by the results obtained by the present invention, and may be confirmed in properly designed clinical trials.

An effective dosage and treatment protocol may be determined by conventional means, starting with a low dose in laboratory animals and then increasing the dosage while monitoring the effects, and systematically varying the dosage regimen as well. Numerous factors may be taken into consideration by a clinician when determining an optimal dosage for a given subject. Such considerations are known to the skilled person.

The term “pharmaceutically acceptable carrier” includes any of the standard pharmaceutical carriers. Pharmaceutically acceptable carriers for therapeutic use are well known in the pharmaceutical art, and are described, for example, in Remington's Pharmaceutical Sciences, Mack Publishing Co. (A. R. Gennaro edit. 1985). For example, sterile saline and phosphate-buffered saline at slightly acidic or physiological pH may be used. pH buffering agents may be phosphate, citrate, acetate, tris/hydroxymethyl)aminomethane (TRIS), N-Tris(hydroxymethyl)methyl-3-aminopropanesulphonic acid (TAPS), ammonium bicarbonate, diethanolamine, histidine, which is a preferred buffer, arginine, lysine, or acetate or mixtures thereof. The term further encompases any agents listed in the US Pharmacopeia for use in animals, including humans.

The term “pharmaceutically acceptable salt” refers to the salt of the compounds. Salts include pharmaceutically acceptable salts such as acid addition salts and basic salts. Examples of acid addition salts include hydrochloride salts, citrate salts and acetate salts. Examples of basic salts include salts where the cation is selected from alkali metals, such as sodium and potassium, alkaline earth metals such as calcium, and ammonium ions ⁺N(R³)₃(R⁴), where R³ and R⁴ independently designates optionally substituted C₁₋₆-alkyl, optionally substituted C₂₋₆-alkenyl, optionally substituted aryl, or optionally substituted heteroaryl. Other examples of pharmaceutically acceptable salts are described in “Remington's Pharmaceutical Sciences”, 17th edition. Ed, Alfonso R. Gennaro (Ed.), Mark Publishing Company, Easton, Pa., U.S.A., 1985 and more recent editions, and in the Encyclopaedia of Pharmaceutical Technology.

“Treatment” is an approach for obtaining beneficial or desired clinical results. For the purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. “Treatment” is an intervention performed with the intention of preventing the development or altering the pathology of a disorder. Accordingly, “treatment” refers to both therapeutic treatment and prophylactic or preventative measures. Those in need of treatment include those already with the disorder as well as those in which the disorder is to be prevented. By treatment is meant inhibiting or reducing an increase in pathology or symptoms (e.g., weight gain, hyperglycaemia) when compared to the absence of treatment, and is not necessarily meant to imply complete cessation of the relevant condition.

The pharmaceutical compositions can be in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms. It may be provided in single dose injectable form, for example in the form of a pen. Compositions may be formulated for any suitable route and means of administration. Pharmaceutically acceptable carriers or diluents include those used in formulations suitable for oral, rectal, nasal or parenteral (including subcutaneous, intramuscular, intravenous, intradermal, and transdermal) administration. The formulations may conveniently be presented in unit dosage form and may be prepared by any of the methods well known in the art of pharmacy.

Subcutaneous or transdermal modes of administration may be particularly suitable for the compounds described herein.

### Combination Therapy

The methods and kits of the invention include administration a combination therapy of a compound described herein with an insulin analog together with a further active agent for treatment of diseases including diabetes, obesity, dyslipidaemia, and hypertension.

In such cases, the three or further active agents may be given together or separately.

Thus the compound of the invention (or the salt thereof) can be used in a further combination with an anti-diabetic agent including but not limited to metformin, a sulfonylurea, a glinide, a DPP-IV inhibitor, a glitazone, or insulin. In a preferred embodiment the compound or salt thereof is used in combination with insulin, DPP-IV inhibitor, sulfonylurea or metformin, particularly sulfonylurea or metformin, for achieving adequate glycemic control.

The compound or salt thereof can further be used in a further combination with an anti-obesity agent including but not limited to a glucagon-like peptide receptor 1 agonist, peptide YY or analogue thereof, cannabinoid receptor 1 antagonist, lipase inhibitor, melanocortin receptor 4 agonist, or melanin concentrating hormone receptor 1 antagonist.

The compound or salt thereof can be used in a further combination with an anti-hypertension agent including but not limited to an angiotensin-converting enzyme inhibitor, angiotensin II receptor blocker, diuretics, beta-blocker, or calcium channel blocker.

The compound or salt thereof can be used in a further combination with an anti-dyslipidaemia agent including but not limited to a statin, a fibrate, a niacin and/or a cholesterol absorbtion inhibitor.

### Methods

### Materials

### Test Substances

| | | Peptide | | |
|---|---|---|---|---|
| | MW | Content | | |
| Drug Name | (g/mol) | Calculated % | Purity % | Solvent |
| Compound X | 3669.2 | 88.9 | 94 | PBS |

PBS: Phosphate buffered saline Gibco (#10010, pH = 7.4). The molar equivalents of peptide used are calculated from the mass of the lyophilized compound, the experimentally determined purity, and the peptide content (calculated or experimentally determined)¹.
¹The equation used to calculate the molar equivalents of peptide is: n_{peptide} = (m_{lyophilized compound} * (% purity/100) * (% peptide content/100))/Mw_{peptide}.

Compound X was produced internally at Zealand Pharma A/S. Lantus (Insulin glargine, Sanofi Aventis) and Levemir (Insulin detemir, Novo Nordisk) were purchased from the local pharmacy (Glostrup Apotek, Denmark), Both insulins are delivered as containers with 3 ml and 100 U/ml. These preparations of insulin are used directly (un-diluted). For dosing of Lantus the standard pen system Optipen is used, with a minimum dosing of 1 U. For dosing of Levemir the pen system Junior demi is used, with a minimum dosing of 0.5 U.

### Animals

Eighty (80) db/db (BKS.Cg-m+/+Lepr^{db}/J) female mice aged 7 weeks were obtained from Charles River, US. The mice were acclimatized in their new environment and allowed free access to normal chow (Altromin 1324, Brogaarden A/S, Gentofte, Denmark) and domestic quality tap water added citric acid to pH ˜3.6, except as indicated. The mice were group-housed with 3-4 mice per cage in a light-, temperature-, and humidity-controlled room (12-hour light: 12-hour dark cycle, with lights on at 06.00 AM to 06.00 PM hour; 24° C.; 50% relative humidity).

### Procedure

Pre-Screen

Prior to treatment, in weeks 1-3, a tail-blood sample for the determination of blood glucose was obtained on non-fasted animals to determine diabetic state and to identify outliers, which were excluded. The inclusion criteria of SG>16 mM glucose was applied.

### Stratification

Stratification of the animals was based on HbA1c levels (primary) and BW as measured at baseline (day −4). Thus, on day −3, the 66 mice were selected based on the pre-screen and baseline measurements into 6 study groups of 11 mice (3-4 mice/cage).

Dosing, Body Weight, Food and Water Intake

All mice were mock treated for at 3 days (BID, SC, 100 μl vehicle) to acclimatize the animals to handling and injections. Dosing (day 0, mice at age 12 weeks as in pilot study) started in the afternoon on that day, and the mice were treated twice daily with 2 SC injections according to Table 1 for a total of 21 days (4 injections per day). Thus, last day of dosing was day 21 in the morning. The daily injections took place between 7:00 and 8:00 and between 14:00 and 15:00 with fresh solutions prepared in the morning (only Compound X). Insulin was kept in the refrigerator.

TABLE 1

| | | | | | Approx. daily |
|---|---|---|---|---|---|
| Groups | | | Substance | Substance | Use of |
| (n = 11/ | | | 1 | 2 | substance |
| group) | Substance | Route | (U/animal) | (nmol/kg) | (mg/day)² |
| Group 1 | Saline + PBS | S.C. | 0 | 0 | — |
| Group 2 | Lantus + PBS | BID | 3 | 0 | — |
| Group 3 | Levemir + PBS | | 6 | 0 | — |
| Group 4 | Saline + | | 0 | 10 | 0.0628 |
| | Compound X | | | | |
| Group 5 | Lantus + | | 3 | 10 | 0.0628 |
| | Compound X | | | | |
| Group 6 | Levemir + | | 6 | 10 | 0.0628 |
| | Compound X | | | | |

S.C = subcutaneous,
BID = bi-daily
Dosing solutions of Compound X for the weekend were prepared the Friday before. One vial was prepared for every dosing. Injection volume (Compound X or PBS): 5 ml/kg. Throughout the study (day −2 to day 21) body weights (BW) were recorded daily and used to administer the body weight-corrected doses of substances. Food and water intake (FI, WI) was measured every day (day −3 to day 21) as cage averages.
²Daily use of substance per day calculated as: Dose (nmol/kg/day) * MW (g/mol) * 0.05 kg/mouse * 11 mouse/group * 1.3 (spill-factor)

### Blood Samples

On day −4 (before starting treatment) in 8 h fasted mice, a blood sample (150 μl) was obtained from the orbital plexus using an EDTA coated micro-pipette taken into EDTA coated tubes kept on ice. From that sample, a drop was used for analysis of blood glucose (BG) (sticks).

Also, 30 μl sample of blood was transferred to a new tube for testing of HbA1c. Stored samples for HbAlc analysis were kept at 4° C. for no more than 48 hours before analysis. The remaining blood was centrifuged, and the resulting plasma (approximately 50 μl) was stored (at −80° C.) for later analysis of plasma insulin level.

On day 21 (before termination) in 8 h fasted mice a blood sample (350 μl) was taken, and BG, HbA1c, and p-insulin were measured as described above. In addition a plasma sample (at least 100 μl) was stored (at −80° C.) for later analysis of exposure.

Termination

The study was terminated on day 21. All animals were sacrificed immediately following the last blood sampling by CO₂ anesthesia followed by cervical dislocation.

### Analysis

The whole blood glucose level was analyzed on tail-blood samples by the immobilized glucose oxidase method (Elite Auto analyser, Bayer, Denmark) following the manufacturer's protocol. Blood samples (sample size 30 μl) were analyzed for HbA1c using the Cobas c111 analyzer (Roche Diagnostics, Mannheim, Germany) in single determinations by Department of Molecular Pharmacology. Plasma (sample size 5 μl) and insulin content was measured using an insulin alpha-LISA assay in triplicate by Department of Molecular Pharmacology. A measure of peptide exposure in plasma (sample size 100 μl) will be determined by the Department of Bioanalysis and Pharmacokinetics.

### Data Analysis

Statistical analyses will be performed using GraphPad Prism version 5. The measured parameters will be compared using a one way and/or two-way ANOVA and relevant post-hoc analyses will be conducted. Differences will be considered statistically significant at p<0.05. Possible outliers will be evaluated by Grubbs outlier test.

### Generation of Cell Lines Expressing Human Glucagon- and GLP-1 Receptors

The cDNA encoding either the human glucagon receptor (Glucagon-R) (primary accession number P47871) or the human glucagon-like peptide 1 receptor (GLP-1R) (primary accession number P43220) were cloned from the cDNA clones BC104854 (MGC:132514/IMAGE:8143857) or BC112126 (MGC:138331/IMAGE:8327594), respectively. The DNA encoding the Glucagon-R or the GLP-1R was amplified by PCR using primers encoding terminal restriction sites for subcloning. The 5′-end primers additionally encoded a near Kozak consensus sequence to ensure efficient translation. The fidelity of the DNA encoding the Glucagon-R and the GLP-1R was confirmed by DNA sequencing. The PCR products encoding the Glucagon-R or the GLP-1R were subcloned into a mammalian expression vector containing a neomycin (G418) resistance marker.

The mammalian expression vectors encoding the Glucagon-R or the GLP-1R were transfected into HEK293 cells by a standard calcium phosphate transfection method. 48 hr after transfection cells were seeded for limited dilution cloning and selected with 1 mg/ml G418 in the culture medium. Three weeks later 12 surviving colonies of Glucagon-R and GLP-1R expressing cells were picked, propagated and tested in the Glucagon-R and GLP-1R efficacy assays as described below. One Glucagon-R expressing clone and one GLP-1R expressing clone were chosen for compound profiling.

### Glucagon Receptor and GLP-1 Receptor Efficacy Assays

HEK293 cells expressing the human Glucagon-R, or human GLP-1R were seeded at 40,000 cells per well in 96-well microtiter plates coated with 0.01% poly-L-lysine and grown for 1 day in culture in 100 μl growth medium. On the day of analysis, growth medium was removed and the cells washed once with 200 μl Tyrode buffer. Cells were incubated in 100 μl Tyrode buffer containing increasing concentrations of test peptides, 100 μM IBMX, and 6 mM glucose for 15 min at 37° C. The reaction was stopped by addition of 25 μl 0.5 M HCl and incubated on ice for 60 min. The cAMP content was estimated using the FlashPlate® cAMP kit from Perkin-Elmer. EC₅₀ and relative efficacies compared to reference compounds (glucagon and GLP-1) were estimated by computer aided curve fitting.

In Vivo: female db/db mice aged 10-11 weeks were treated for 21 days with bi-daily s.c. injections. Groups: vehicle (PBS), Lantus (3 U), Levemir (6 U), COMPOUND X (10 nmol/kg), Lantus (3 U)+COMPOUND X (10 nmol/kg), Levemir (6 U)+COMPOUND X (10 nmol/kg). Fasting blood glucose (BG) was measured before and after 21 days of treatment.

### EXAMPLES

### Example 1

### Reduction of Weight Gain by the Compound Compound X in Mice Receiving Insulin Analogues

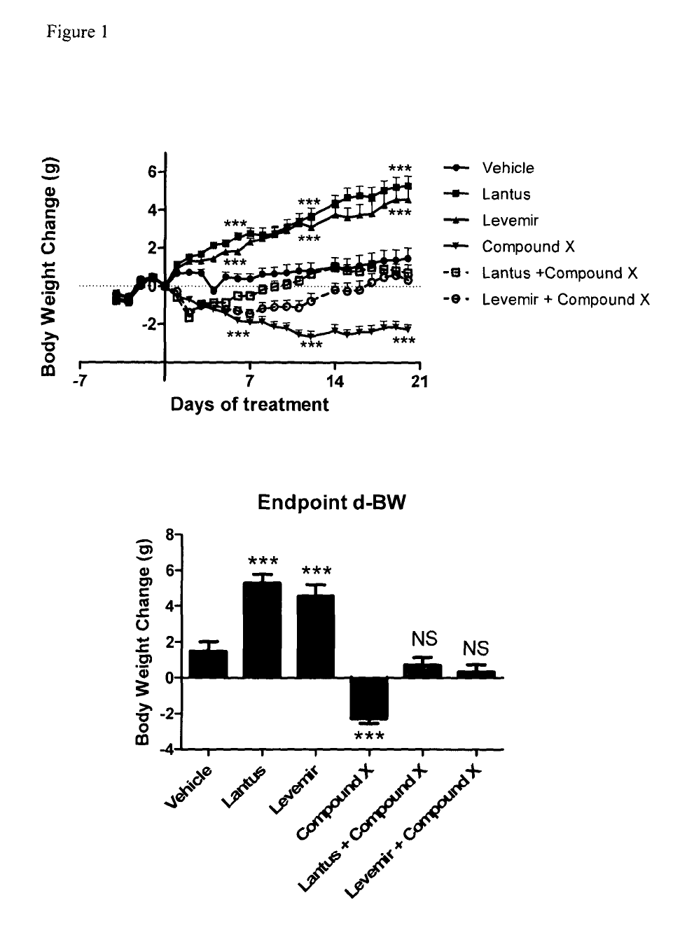

As shown in FIG. 1, we observed a significant increase in body weight in mice treated with either Lantus or Levemir, while treatment with Compound X caused a significant decrease in BW. Interestingly, BW in mice treated with both Compound X and Lantus or Levemir was similar to that of vehicle control. Our results indicate that combination of a long-acting insulin and GluGLP-1 dual agonist Compound X may improve glycemic control while avoiding the undesirable weight gain of conventional insulin treatment, or promote a overall weight-loss while improving glycemic control.

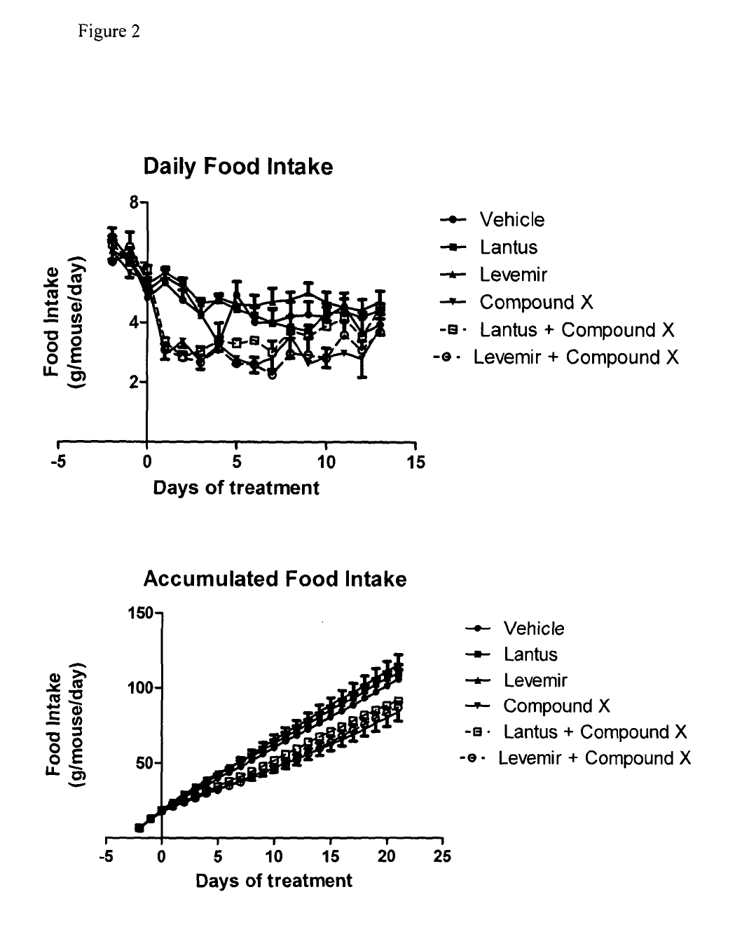

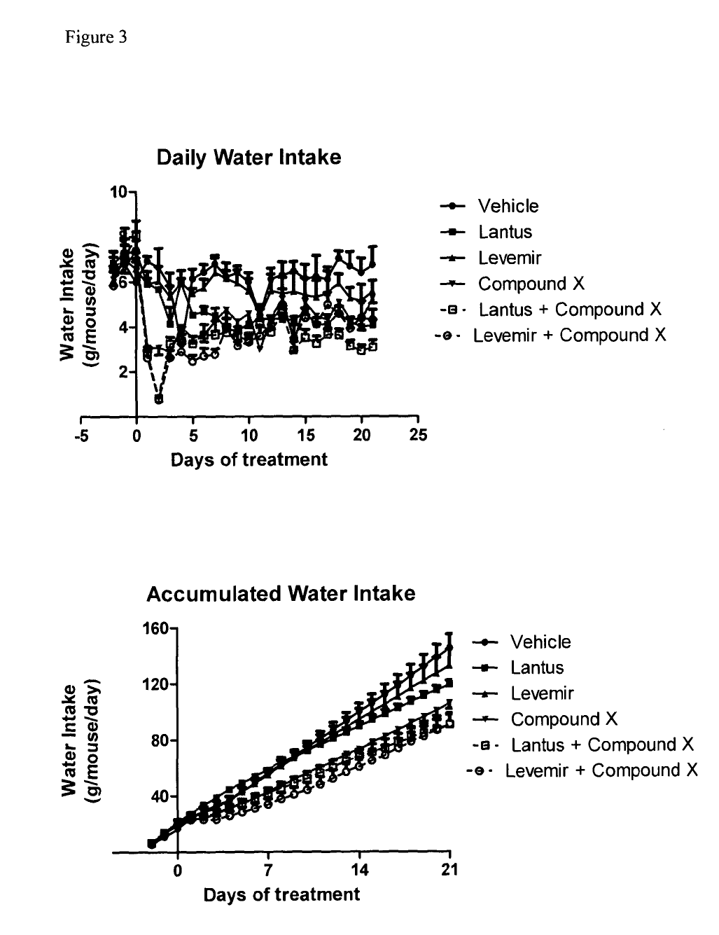

Food intake was reduced in mice receiving Compound X in combination with either Lantus or Levemir as compared to mice receiving Lantus or Levemir alone alone, as shown in FIG. 2. Similarly, intake of water in mice receiving Compound X combination with either Lantus or Levemir was reduced, as compared to mice receiving either Latnus or Levemire alone. These results are shown in FIG. 3.

### Example 2

### Efficacy on GLP-1 and Glucagon Receptors

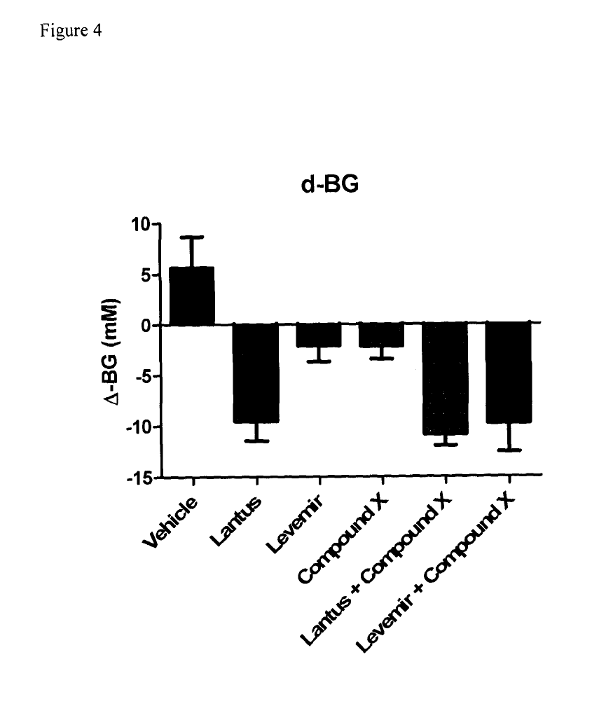

FIG. 4 shows the delta-BG. When mice were treated with Lantus alone or in combination with the glucagon-GLP-1 dual agonist Compound X, in contrast to vehicle control we observed a decrease in delta-BG over the course of the 21-day experiment (mM, −9.6±1.9 vs. −10.9±1.1, Lantus vs. Lantus+Compound X; p=ns). In animals treated with Levemir, we also observed a decrease in delta-BG, which was more pronounced when combined with Compound X (mM, −2.1±1.6 vs. −9.8±2.8, Levemir vs. Levemir+Compound X, p<0.05).

### Other Embodiments

From the foregoing description, it will be apparent that variations and modifications may be made to the invention described herein to adopt it to various usages and conditions. Such embodiments are also within the scope of the following claims.

All publications, patent applications, and patents mentioned in this specification are herein incorporated by reference to the same extent as if each independent publication, patent application, or patent was specifically and individually indicated to be incorporated by reference.

1-4. (canceled)

5. A method for preventing or reducing weight gain; promoting weight loss; improving circulating glucose levels, glucose tolerance or circulating cholesterol levels; lowering circulating LDL levels; increasing HDL/LDL ratio; or treating a condition caused or characterized by excess body weight, said method comprising administering to a mammalian subject a combination of compounds comprising:
(a) a compound having the formula:
R¹—Z—R²
wherein R¹ is H, C₁₋₄ alkyl, acetyl, formyl, benzoyl, or trifluoroacetyl;
R² is OH or NH₂;
and Z is a peptide having the formula I:
His-X2-Gln-Gly-Thr-Phe-Thr-Ser-Asp-Tyr-Ser-X12-Tyr-Leu-Asp-X16-X17-Ala-Ala-X20-X21-Phe-Val-X24-Trp-Leu-X27-X28-Ala-X30 (SEQ ID NO:6); (I)
wherein
X2 is selected from Aib and Ser;
X12 is selected from Lys, Arg, or Leu;
X16 is selected from Arg and X;
X17 is selected from Arg and X;
X20 is selected from Arg, His, and X;
X21 is selected from Asp and Glu;
X24 is selected from Ala and X;
X27 is selected from Leu and X;
X28 is selected from Arg and X;
X30 is X or is absent;
wherein at least one of X16, X17, X20, X24, X27, X28, and X30 is X;
and wherein each residue X is independently selected from the group consisting of Glu, Lys, Ser, Cys, Dbu, Dpr, and Orn;
wherein the side chain of at least one residue X is conjugated to a lipophilic substituent having the formula:
(i) Z¹, wherein Z¹ is a lipophilic moiety conjugated directly to the side chain of X; or
(ii) Z¹Z², wherein Z¹ is a lipophilic moiety, Z² is a spacer, and Z¹ is conjugated to the side chain of X via Z²; and
(b) an insulin analogue.

6. The method of claim 5, wherein said insulin analogue is selected from the group consisting of insulin glulisine, insulin lispro, Degludec, Actraphane HM, LY2963016, LY2605541, pegylated insulin Lispro, insulin glargine, insulin detemir, insulin isophane, insulin aspart, insulin buccal, hyaluronidase insulin, insulin protamine, NN-1953, IN-105, BIOD-620, and Analog-PH20.

7-8. (canceled)

9. The method of claim 5, wherein said condition caused or characterized by excess body weight is selected from the group consisting of obesity, morbid obesity, obesity-linked inflammation, obesity-linked gallbladder disease, obesity-induced sleep apnea, metabolic syndrome, pre-diabetes, insulin resistance, glucose intolerance, type 2 diabetes, type I diabetes, hypertension, atherogenic dyslipidaemia, atherosclerosis, arteriosclerosis, coronary heart disease, peripheral artery disease, stroke, and microvascular disease.

10-11. (canceled)

12. The method of claim 5, wherein said subject has type 1 or type 2 diabetes.

13. The method of claim 5, wherein one or more of said residues X is independently selected from Lys, Glu and Cys.

14. The method of claim 5, wherein:
X16 is selected from Glu, Lys, and Ser;
X17 is selected from Lys and Cys;
X20 is selected from His, Lys, Arg, and Cys;
X24 is selected from Lys, Glu, and Ala,
X27 is selected from Leu and Lys; and/or
X28 is selected from Ser, Arg, and Lys.

15. The method of claim 5, wherein the peptide of formula I includes one or more of a combination of residues selected from the group consisting of:
X2 is Aib and X17 is Lys;
X2 is Aib and X17 is Cys;
X2 is Aib and X20 is Cys;
X2 is Aib and X28 is Lys;
X12 is Arg and X17 is Lys
X12 is Leu and X17 is Lys
X12 is Lys and X20 is Lys
X12 is Lys and X17 is Lys
X16 is Lys and X17 is Lys
X16 is Ser and X17 is Lys,
X17 is Lys and X20 is Lys
X17 is Lys and X21 is Asp
X17 is Lys and X24 is Glu
X17 is Lys and X27 is Leu
X17 is Lys and X27 is Lys
X17 is Lys and X28 is Ser
X17 is Lys and X28 is Arg
X20 is Lys and X27 is Leu
X21 is Asp and X27 is Leu
X2 is Aib, X2 is Lys, and X16 is Ser;
X12 is Lys, X17 is Lys, and X16 is Ser;
X12 is Arg, X17 is Lys, and X16 is Glu;
X16 is Glu, X17 is Lys, and X20 is Lys;
X16 is Ser, X21 is Asp, and X24 is Glu;
X17 is Lys, X24 is Glu, and X28 is Arg;
X17 is Lys, X24 is Glu, and X28 is Lys;
X17 is Lys, X27 is Leu, and X28 is Ser;
X17 is Lys, X27 is Leu, and X28 is Arg;
X20 is Lys, X24 is Glu, and X27 is Leu;
X20 is Lys, X27 is Leu, and X28 is Ser;
X20 is Lys, X27 is Leu, and X28 is Arg;
X16 is Ser, X20 is His, X24 is Glu, and X27 is Leu;
X17 is Lys, X20 is His, X24 is Glu, and X28 is Ser;
X17 is Lys, X20 is Lys, X24 is Glu, and X27 is Leu; and
X17 is Cys, X20 is Lys, X24 is Glu, and X27 is Leu.

16. The method of claim 5, wherein the peptide of formula I contains only one amino acid of the type conjugated to the lipophilic substituent.

17. The method of claim 5, wherein the peptide of formula I contains only one Lys residue, only one Cys residue, or only one Glu residue, and wherein the lipophilic substituent is conjugated to that residue.

18. The method of claim 5, wherein the peptide sequence of formula I comprises one or more intramolecular bridges.

19. The method of claim 18, wherein the intramolecular bridge is selected from the group consisting of:
a.) an intramolecular bridge formed between the side chains of two amino acid residues which are separated by three amino acids in the linear amino acid sequence of formula I,
b.) an intramolecular bridge formed between the side chains of residue pairs 16 and 20, 17 and 21, 20 and 24, or 24 and 28,
c.) a salt bridge or a lactam ring, and
d.) an intramolecular bridge which involves a pair of residues selected from the group consisting of:
X16 is Glu and X20 is Lys;
X16 is Glu and X20 is Arg,
X16 is Lys and X20 is Glu;
X16 is Arg and X20 is Glu;
X17 is Arg and X21 is Glu;
X17 is Lys and X21 is Glu,
X17 is Arg and X21 is Asp;
X17 is Lys and X21 is Asp;
X20 is Glu and X24 is Lys;
X20 is Glu and X24 is Arg;
X20 is Lys and X24 is Glu;
X20 is Arg and X24 is Glu;
X24 is Glu and X28 is Lys;
X24 is Glu and X28 is Arg;
X24 is Lys and X28 is Glu; and
X24 is Arg and X28 is Glu.

20-22. (canceled)

23. The method of claim 5, wherein at least one of X16, X17, X20, and X28 is conjugated to a lipophilic substituent.

24. The method of claim 5, wherein X30 is present and conjugated to a lipophilic substituent.

25. (canceled)

26. The method of claim 5, wherein the compound has lipophilic substituents selected from the group consisting of:
a.) just one lipophilic substituent, at position 16, 17, 20, 24, 27, 28 or 30, preferably at position 16, 17 or 20, particularly at position 17
b.) precisely two lipophilic substituents, each at one of positions 16, 17, 20, 24, 27, 28, and 30, and
c.) lipophilic substituents at positions 16 and 17, 16 and 20, 16 and 24, 16 and 27, 16 and 28, 16 and 30, 17 and 20, 17 and 24, 17 and 27, 17 and 28, 17 and 30, 20 and 24, 20 and 27, 20 and 28, 20 and 30, 24 and 27, 24 and 28, 24 and 30, 27 and 28, 27 and 30, or 28 and 30.

27-28. (canceled)

29. The method of claim 5, wherein said compound has the formula:
R¹—Z—R²
wherein R¹ is H, C₁₋₄ alkyl, acetyl, formyl, benzoyl, or trifluoroacetyl;
R² is OH or NH₂;
and Z is selected from the group consisting of:
a.) a peptide having the formula IIa:
His-Aib-Gln-Gly-Thr-Phe-Thr-Ser-Asp-Tyr-Ser-X12-Tyr-Leu-Asp-X16-X17-Ala-Ala-X20-X21-Phe-Val-X24-Trp-Leu-Leu-X28-Ala (SEQ ID NO:7); (IIa)
wherein:
X12 is selected from Lys, Arg, and Leu;
X16 is selected from Ser and X;
X17 is X;
X20 is selected from His and X,
X21 is selected from Asp and Glu;
X24 is selected from Ala and Glu;
X28 is selected from Ser, Lys, and Arg;
and wherein each residue X is independently selected from the group consisting of Glu, Lys, and Cys;
wherein the side chain of at least one residue X is conjugated to a lipophilic substituent having the formula:
(i) Z¹ wherein Z¹ is a lipophilic moiety conjugated directly to the side chain of X; or
(ii) Z¹Z², wherein Z¹ is a lipophilic moiety, Z² is a spacer, and Z¹ is conjugated to the side chain of X via Z²,
b.) a peptide having the formula IIIa:
His-Aib-Gln-Gly-Thr-Phe-Thr-Ser-Asp-Tyr-Ser-X12-Tyr-Leu-Asp-Ser-X17-Ala-Ala-X20-X21-Phe-Val-X24-Trp-Leu-Leu-X28-Ala; (IIIa)
wherein:
X12 is selected from Lys And Arg;
X17 is X;
X20 is selected from His and X;
X21 is selected from Asp and Glu;
X24 is selected from Ala and Glu;
X28 is selected from Ser, Lys, and Arg;
and wherein each residue X is independently selected from Glu, Lys, and Cys;
wherein the side chain of at least one residue X is conjugated to a lipophilic substituent having the formula:
(i), wherein Z¹ is a lipophilic moiety conjugated directly to the side chain of X; or
(ii) Z¹Z², wherein Z¹ is a lipophilic moiety, Z² is a spacer, and Z¹ is conjugated to the side chain of X via Z², and
c.) a peptide having the formula IVa:
His-Aib-Gln-Gly-Thr-Phe-Thr-Ser-Asp-Tyr-Ser-X12-Tyr-Leu-Asp-Ser-X17-Ala-Ala-His-X21-Phe-Val-X24-Trp-Leu-Leu-X28-Ala; (IVa):
wherein
X12 is selected from Lys and Arg;
X17 is X;
X21 is selected from Asp and Glu;
X24 is selected from Ala and Glu:
X28 is selected from Ser, Lys, and Arg,
wherein X is selected from the group consisting of Glu, Lys, and Cys;
and wherein the side chain of X is conjugated to a lipophilic substituent having the formula:
(i) Z¹, wherein Z¹ is a lipophilic moiety conjugated directly to the side chain of X; or
(4) Z¹Z², wherein Z¹ is a lipophilic moiety, Z² is a spacer, and Z¹ is conjugated to the side chain of X via Z².

30-34. (canceled)

35. The method of claim 5, wherein said peptide Z has a sequence selected from the group consisting of:
(SEQ ID NO: 13)
HSQGTFTSDYSKYLDSKAAHDFVEWLLRA;
(SEQ ID NO: 14)
HSQGTFTSDYSKYLDKKAAHDFVEWLLRA;
(SEQ ID NO: 15)
HSQGTFTSDYSKYLDSKAAKDFVEWLLRA;
(SEQ ID NO: 16)
HSQGTFTSDYSKYLDSKAAHDFVEWLKRA;
(SEQ ID NO: 17)
HSQGTFTSDYSKYLDSKAAHDFVEWLLKA;
(SEQ ID NO: 18)
HSQGTFTSDYSRYLDSKAAHDFVEWLLRA;
(SEQ ID NO: 19)
HSQGTFTSDYSLYLDSKAAHDFVEWLLRA;
(SEQ ID NO: 20)
HSQGTFTSDYSKYLDSKAAHDFVEWLLRAK;
(SEQ ID NO: 21)
HSQGTFTSDYSKYLDSKAAHDFVEWLLSAK;
(SEQ ID NO: 22)
HSQGTFTSDYSKYLDSKAAHDFVEWLKSA;
(SEQ ID NO: 23)
HSQGTFTSDYSKYLDSKAAHDFVKWLLRA;
(SEQ ID NO: 24)
HSQGTFTSDYSKYLDSCAAHDFVEWLLRA;
(SEQ ID NO: 25)
HSQGTFTSDYSKYLDSCAAHDFVEWLLSA;
(SEQ ID NO: 26)
HSQGTFTSDYSKYLDSKAACDFVEWLLRA;
(SEQ ID NO: 27)
HSQGTFTSDYSKYLDKSAAHDFVEWLLRA;
(SEQ ID NO: 28)
H-Aib-QGTFTSDYSKYLDSKAAHDFVEWLLSA;
(SEQ ID NO: 29)
H-Aib-QGTFTSDYSKYLDSKAAHDFVEWLLSAK;
(SEQ ID NO: 30)
H-Aib-QGTFTSDYSKYLDSKAARDFVAWLLRA;
(SEQ ID NO: 31)
H-Aib-QGTFTSDYSKYLDSKAAKDFVAWLLRA;
(SEQ ID NO: 32)
H-Aib-QGTFTSDYSKYLDSKAAHDFVEWLLRA;
(SEQ ID NO: 33)
H-Aib-QGTFTSDYSKYLDSKAAHDFVEWLLKA;
(SEQ ID NO: 34)
H-Aib-QGTFTSDYSKYLDSKAAKDFVAWLLSA;
(SEQ ID NO: 35)
H-Aib-QGTFTSDYSKYLDSKAAHDFVAWLLKA;
(SEQ ID NO: 36)
H-Aib-QGTFTSDYSKYLDKKAAHDFVAWLLRA;
(SEQ ID NO: 37)
H-Aib-QGTFTSDYSRYLDSKAAHDFVEWLLSA;
(SEQ ID NO: 38)
H-Aib-QGTFTSDYSKYLDSKAAHDFVKWLLSA;
(SEQ ID NO: 39)
H-Aib-QGTFTSDYSLYLDSKAAHDFVEWLLSA;
(SEQ ID NO: 40)
H-Aib-QGTFTSDYSKYLDSCAAHDFVEWLLSA;
(SEQ ID NO: 41)
H-Aib-QGTFTSDYSKYLDSKAACDFVEWLLRA;
(SEQ ID NO: 42)
H-Aib-QGTFTSDYSKYLDK()KAAE()DFVEWLLRA;
(SEQ ID NO: 43)
H-Aib-QGTFTSDYSKYLDSKAAHDFVE()WLLK()A;
(SEQ ID NO: 44)
H-Aib-QGTFTSDYSKYLDSKAAK()DFVE()WLLRA;
(SEQ ID NO: 45)
H-Aib-QGTFTSDYSKYLDSK()AAHE()FVEWLLKA;
(SEQ ID NO: 46)
H-Aib-QGTFTSDYSKYLDSK()AAKE()FVEWLLRA;
(SEQ ID NO: 47)
HSQGTFTSDYSKYLDS-K*-AAHDFVEWLLRA;
(SEQ ID NO: 48)
HSQGTFTSDYSKYLD-K*-KAAHDFVEWLLRA;
(SEQ ID NO: 49)
HSQGTFTSDYSKYLDSKAA-K*-DFVEWLLRA;
(SEQ ID NO: 50)
HSQGTFTSDYSKYLDSKAAHDFVEWL-K*-RA;
(SEQ ID NO: 51)
HSQGTFTSDYSKYLDSKAAHDFVEWLL-K*-A;
(SEQ ID NO: 52)
HSQGTFTSDYSRYLDS-K*-AAHDFVEWLLRA;
(SEQ ID NO: 53)
HSQGTFTSDYSLYLDS-K*-AAHDFVEWLLRA;
(SEQ ID NO: 54)
HSQGTFTSDYSKYLDSKAAHDFVEWLLRA-K*;
(SEQ ID NO: 55)
HSQGTFTSDYSKYLDSKAAHDFVEWLLSA-K*;
(SEQ ID NO: 56)
HSQGTFTSDYSKYLDSKAAHDFVEWL-K*-SA;
(SEQ ID NO: 57)
HSQGTFTSDYSKYLDSKAAHDFV-K*-WLLRA;
(SEQ ID NO: 58)
HSQGTFTSDYSKYLDS-C*-AAHDFVEWLLRA;
(SEQ ID NO: 59)
HSQGTFTSDYSKYLDS-C*-AAHDFVEWLLSA;
(SEQ ID NO: 60)
HSQGTFTSDYSKYLDSKAA-C*-DFVEWLLRA;
(SEQ ID NO: 61)
HSQGTFTSDYSKYLD-K*-SAAHDFVEWLLRA;
(SEQ ID NO: 62)
H-Aib-QGTFTSDYSKYLDS-K*-AAHDFVEWLLSA;
(SEQ ID NO: 63)
H-Aib-QGTFTSDYSKYLDSKAAHDFVEWLLSA-K*;
(SEQ ID NO: 64)
H-Aib-QGTFTSDYSKYLDS-K*-AARDFVAWLLRA;
(SEQ ID NO: 65)
H-Aib-QGTFTSDYSKYLDSKAA-K*-DFVAWLLRA;
(SEQ ID NO: 66)
H-Aib-QGTFTSDYSKYLDSKAAHDFVEWLL-K*-A;
(SEQ ID NO: 67)
H-Aib-QGTFTSDYSKYLDS-K*-AAHDFVEWLLRA;
(SEQ ID NO: 68)
H-Aib-QGTFTSDYSKYLDS-K*-AAHDFVEWLLKA;
(SEQ ID NO: 69)
H-Aib-QGTFTSDYSKYLDSKAA-K*-DFVAWLLSA;
(SEQ ID NO: 70)
H-Aib-QGTFTSDYSKYLDSKAAHDFVAWLL-K*-A;
(SEQ ID NO: 71)
H-Aib-QGTFTSDYSKYLD-K*-KAAHDFVAWLLRA;
(SEQ ID NO: 72)
H-Aib-QGTFTSDYSRYLDS-K*-AAHDFVEWLLSA;
(SEQ ID NO: 73)
H-Aib-QGTFTSDYSKYLDSKAAHDFV-K*-WLLSA;
(SEQ ID NO: 74)
H-Aib-QGTFTSDYSLYLDS-K*-AAHDFVEWLLSA;
(SEQ ID NO: 75)
H-Aib-QGTFTSDYSKYLDS-C*-AAHDFVEWLLSA;
(SEQ ID NO: 76)
H-Aib-QGTFTSDYSKYLDSKAA-C*-DFVEWLLRA;
(SEQ ID NO: 77)
H-Aib-QGTFTSDYSKYLD-S*-KAAHDFVEWLLSA;
(SEQ ID NO: 78)
H-Aib-QGTFTSDYSKYLDK()K*AAE()DFVEWLLRA;
(SEQ ID NO: 79)
H-Aib-QGTFTSDYSKYLDSK*AAHDFVE()WLLK()A;
(SEQ ID NO: 80)
H-Aib-QGTFTSDYSKYLDSK*AAK()DFVE()WLLRA;
(SEQ ID NO: 81)
H-Aib-QGTFTSDYSKYLDSK()AAHE()FVEWLLK*A;
and
(SEQ ID NO: 82)
H-Aib-QGTFTSDYSKYLDSK()AAK*E()FVEWLLRA,
wherein “*” indicates the position of a
lipophilic substituent.

36. (canceled)

37. The method of claim 5, wherein Z¹ comprises a moiety selected from the group consisting of a hydrocarbon chain having 10 to 24 C atoms, 10 to 22 C atoms, or 10 to 20 C atoms, a dodecanoyl moiety, 2-butyloctanoyl moiety, tetradecanoyl moiety, hexadecanoyl moiety, heptadecanoyl moiety, octadecanoyl moiety, and an eicosanoyl moiety.

38. (canceled)

39. The method of claim 5, wherein Z² is or comprises a moiety selected from the group consisting of one or more amino acid residues, a γ-Glu residue, Glu residue, β-Ala residue, ε-Lys residue, 3-aminopropanoyl moiety, 4-aminobutanoyl moiety, 8-aminooctanoyl moiety, and 8-amino-3,6-dioxaoctanoyl moiety.

40. (canceled)

41. The method of claim 39, wherein the lipophilic substituent is selected from the group consisting of dodecanoyl-γ-Glu, hexadecanoly-γ-Glu, hexadecanoyl-Glu, hexadecanoyl-[3-aminopropanoyl], hexadecanoyl-[8-aminooctanoyl], hexadecanoyl-ε-Lys, 2-butyloctanoyl-γ-Glu, octadecanoyl-γ-Glu, and hexadecanoyl-[4-aminobutanoyl].

42. The method of claim 41, wherein Z has a formula selected from the group consisting of:
(SEQ ID NO: 83)
HSQGTFTSDYSKYLD-K(Hexadecanoyl-γ-Glu)-KAAHDFVEWLLRA;
(SEQ ID NO: 84)
HSQGTFTSDYSKYLDSKAAHDFVEWL-K(Hexadecanoyl-γ-Glu)-RA;
(SEQ ID NO: 85)
HSQGTFTSDYSKYLDSKAA-K(Hexadecanoyl-γ-Glu)-DFVEWLLRA;
(SEQ ID NO: 86)
HSQGTFTSDYSKYLDSKAAHDFVEWLL-K(Hexadecanoyl-γ-Glu)-A;
(SEQ ID NO: 87)
H-Aib-QGTFTSDYSKYLDS-K(Hexadecanoyl-γ-Glu)-AAHDFVEWLLRA;
(SEQ ID NO: 88)
H-Aib-QGTFTSDYSKYLDS-K(Hexadecanoyl-γ-Glu)-AARDFVAWLLRA;
(SEQ ID NO: 89)
H-Aib-QGTFTSDYSKYLDS-K(Hexadecanoyl-γ-Glu)-AAHDFVEWLLSA;
(SEQ ID NO: 90)
H-Aib-QGTFTSDYSKYLDSKAAHDFVEWLL-K(Hexadecanoyl-γ-Glu)-A;
(SEQ ID NO: 91)
H-Aib-QGTFTSDYSKYLDS-K(Hexadecanoyl-γ-Glu)-AAHDFVEWLLKA;
(SEQ ID NO: 92)
H-Aib-QGTFTSDYSKYLDS-K(Hexadecanoyl-γ-Glu)-AAHDFVE()WLLK()A;
(SEQ ID NO: 93)
HSQGTFTSDYSKYLDS-K(Hexadecanoyl-γ-Glu)-AAHDFVEWLLRA;
(SEQ ID NO: 94)
H-Aib-QGTFTSDYSKYLDSKAA-K(Hexadecanoyl-γ-Glu)-DFVAWLLRA;
(SEQ ID NO: 95)
H-Aib-QGTFTSDYSKYLDS-K(Dodecanoyl-γ-Glu)-AAH DFVEWLLSA;
(SEQ ID NO: 96)
H-Aib-QGTFTSDYSKYLDS-K(Hexadecanoyl-[3-aminopropanoyl])-AAHDFVEWLLSA;
(SEQ ID NO: 97)
H-Aib-QGTFTSDYSKYLDS-K(Hexadecanoyl-[8-aminooctanoyl])-AAHDFVEWLLSA;
(SEQ ID NO: 98)
H-Aib-QGTFTSDYSKYLDS-K(Hexadecanoyl-ε-Lys)-AAHDFVEWLLSA;
(SEQ ID NO: 99)
HSQGTFTSDYSKYLDS-K(Hexadecanoyl)-AAHDFVEWLLSA;
(SEQ ID NO: 100)
HSQGTFTSDYSKYLDS-K(Octadecanoyl-γ-Glu)-AAHDFVEWLLSA;
(SEQ ID NO: 101)
HSQGTFTSDYSKYLDS-K([2-Butyloctanoyl]-γ-Glu)-AAHDFVEWLLSA;
(SEQ ID NO: 102)
HSQGTFTSDYSKYLDS-K(Hexadecanoyl-[4-Aminobutanoyl])-AAHDFVEWLLSA;
(SEQ ID NO: 103)
HSQGTFTSDYSKYLDS-K(Octadecanoyl-γ-Glu)-AAHDFVEWLLSA;
(SEQ ID NO: 104)
HSQGTFTSDYSKYLDS-K(Hexadecanoyl-E)-AAHDFVEWLLSA;
(SEQ ID NO: 105)
H-Aib-QGTFTSDYSKYLDS-K(Hexadecanoyl)-AAHDFVEWLLSA;
(SEQ ID NO: 106)
H-Aib-QGTFTSDYSKYLDS-K(Octadecanoyl-γ-Glu)-AAHDFVEWLLSA;
(SEQ ID NO: 107)
H-Aib-QGTFTSDYSKYLDS-K([2-Butyloctanoyl]-γ-Glu)-AAHDFVEWLLSA;
(SEQ ID NO: 108)
H-Aib-QGTFTSDYSKYLDS-K(Hexadecanoyl-[4-Aminobutanoyl])-AAHDFVEWLLSA;
(SEQ ID NO: 109)
H-Aib-QGTFTSDYSKYLDS-K(Octadecanoyl-γ-Glu)-AAHDFVEWLLSA;
and
(SEQ ID NO: 110)
H-Aib-QGTFTSDYSKYLDS-K(Hexadecanoyl-E)-AAHDFVEWLLSA;
wherein residues marked “()” participate in an intramolecular bond;
(SEQ ID NO: 111)
H-Aib-QGTFTSDYS-K(Hexadecanoyl-isoGlu)-YLDSKAAHDFVEWLLSA;
(SEQ ID NO: 112)
H-Aib-QGTFTSDYSKYLD-K(Hexadecanoyl-isoGlu)-KAAHDFVEWLLSA;
(SEQ ID NO: 113)
H-Aib-QGTFTSDYSKYLDSKAA-K(Hexadecanoyl-isoGlu)-DFVEWLLSA;
(SEQ ID NO: 114)
H-Aib-QGTFTSDYSKYLDSKAAHDFV-K(Hexadecanoyl-isoGlu)-WLLSA;
(SEQ ID NO: 115)
H-Aib-QGTFTSDYSKYLDS-K(Hexadecanoyl-isoLys)-AARDFVAWLLRA;
(SEQ ID NO: 116)
H-Aib-QGTFTSDYSKYLDS-K(Hexadecanoyl-isoGlu)-AAKDFVEWLLSA;
(SEQ ID NO: 117)
H-Aib-QGTFTSDYSKYLDE-K(Hexadecanoyl-isoGlu)-AAHDFVEWLLSA;
(SEQ ID NO: 118)
H-Aib-QGTFTSDYSKYLDS-K(Hexadecanoyl-isoGlu)-AAHEFVEWLLSA;
(SEQ ID NO: 119)
H-Aib-QGTFTSDYSKYLDS-K(Hexadecanoyl-isoGlu)-AAEDFVEWLLSA,
and
(SEQ ID NO: 120)
H-Aib-QGTFTSDYSKYLDS-K(Hexadecanoyl-isoGlu)-AAHDFVEWLLEA.

43-70. (canceled)

71. The method of claim 5, wherein said combination of compounds is selected from the group consisting of: H—H-Aib-QGTFTSDYSKYLDS-K(Hexadecanoyl-isoGlu)-AAHDFVEWLLSA-NH₂ (SEQ ID NO:126) and insulin glargine, H—H-Aib-QGTFTSDYSKYLDS-K(Hexadecanoyl-isoGlu)-AAHDFVEWLLSA-(SEQ ID NO:126) and insulin detemir, H—H-Aib-QGTFTSDYSKYLDS-K(Hexadecanoyl-isoGlu)-AAHDFVEWLLSA-NH, (SEQ ID NO:126) and glulisine, H—H-Aib-QGTFTSDYSKYLDS-K(Hexadecanoyl-isoGlu)-AAHDFVEWLLSA-NH₂ (SEQ ID NO:126) and insulin lispro, H—H-Aib-QGTFTSDYSKYLDS-K(Hexadecanoyl-isoGlu)-AAHDFVEWLLSA-NH₂ (SEQ ID NO:126) and degludec, H—H-Aib-QGTFTSDYSKYLDS-K(Hexadecanoyl-isoGlu)-AAHDFVEWLLSA-NH₂ (SEQ ID NO:126) and Actraphane, H—H-Aib-QGTFTSDYSKYLDS-K(Hexadecanoyl-isoGlu)-AAHDFVEWLLSA-NH₂ (SEQ ID NO:126) and LY2963016, H—H-Aib-QGTFTSDYSKYLDS-K(Hexadecanoyl-isoGlu)-AAHDFVEWLLSA-NH₂ (SEQ ID NO:1261 and LY2605541, H—H-Aib-QGTFTSDYSKYLDS-K(Hexadecanoyl-isoGlu)-AAHDFVEWLLSA-NH₂ (SEQ ID NO:126) and pegylated insulin Lispro, and H—H-Aib-QGTFTSDYSKYLDS-K(Hexadecanoyl-isoGlu)-AAHDFVEWLLSA-NH₂ (SEQ ID NO:126) and NN-1953, IN-105, BIOD-620 and Analog-PH20.

72-92. (canceled)

93. The method of claim 5, wherein said compound having the formula R¹—Z—R² is administered in a dosage selected from the group consisting of 0.1 nmol/kg to 1 μmol/kg and 3 nmol/kg to 30 nmol/kg.

94. (canceled)

95. The method of claim 5, wherein said insulin analogue is administered in a dosage selected from the group consisting of 0.02 U/kg to 20 U/kg, 0.1 U/kg to 0.3 U/kg, and about 0.2 U/kg.

96-98. (canceled)

99. The method of claim 5, wherein said compound or combination of compounds is administered in an amount selected from:
an amount sufficient to reduce food intake in said subject by at least 5%, 10%, 15%, 20%, 25%, 30%, or 50%,
an amount sufficient to reduce the subject's fasting blood glucose level by at least 1, 2, 3, 4, 5, 6, 8, 10, 11, 12, 15, or 20 mM,
an amount sufficient to reduce the subjects HbA1c level by at least 0.1%, 0.2%, 0.3%, 0.4%, 0.5%, 0.6%, 0.8%, 1.0%, 1.5%, or 2.0%,
an amount which results in a body weight reduction of at least 3%, 5%, 8%, 10%, 12%, 15% or 20% within 1 year of starting administration,
an amount which results in a body weight reduction of at least 1%, 2%, 3%, 4%, 5%, 6%, 8%, or 10%, 15% within six months of starting administration, and
an amount which results in a body weight reduction of at least 0.5%, 1%, 2%, 3%, 4%, 5%, 6%, 8%, 10% or 15% within three months of starting administration.

100-108. (canceled)

109. A kit comprising:
(a) a compound having the formula:
R¹—Z—R²
wherein R¹ is H, C₁₋₄ alkyl, acetyl, formyl, benzoyl, or trifluoroacetyl;
R² is OH or NH₂,
and Z is a peptide having the formula I:
His-X2-Gln-Gly-Thr-Phe-Thr-Ser-Asp-Tyr-Ser-X12-Tyr-Leu-Asp-X16-X17-Ala-Ala-X20-X21-Phe-Val-X24-Trp-Leu-X27-X28-Ala-X30; (I)
wherein
X2 is selected from Aib and Ser;
X12 is selected from Lys, Arg, or Leu;
X16 is selected from Arg and X;
X17 is selected from Arg and X;
X20 is selected from Arg, His, and X;
X21 is selected from Asp and Glu;
X24 is selected from Ala and X;
X27 is selected from Leu and X;
X28 is selected from Arg and X;
X30 is X or is absent;
wherein at least one of X16, X17, X20, X24, X27, X28, and X30 is X;
and wherein each residue X is independently selected from the group consisting of Glu, Lys, Ser, Cys, Dbu, Dpr, and Orn;
wherein the side chain of at least one residue X is conjugated to a lipophilic substituent having the formula:
(i) Z¹, wherein Z¹ is a lipophilic moiety conjugated directly to the side chain of X; or
(ii) Z¹Z², wherein Z¹ is a lipophilic moiety, Z² is a spacer, and Z¹ is conjugated to the side chain of X via Z²; and
(b) an insulin analogue.

110. (canceled)

111. The kit of claim 109, wherein said insulin analogue is selected from the group consisting of insulin glulisine, insulin lispro, Degludec, Actraphane HM, LY2963016, LY2605541, pegylated insulin Lispro, insulin glargine (Lantus) insulin detemir (Levemir), insulin isophane, insulin aspart, insulin buccal, hyaluronidase insulin, insulin protamine, NN-1953, IN-105, BIOD-620 and Analog-PH20.

112. The method of claim 5, wherein the compound (a) an insulin analogue (b) are formulated for simultaneous or sequential administration.

113. The method of claim 5, wherein the compound (a) and insulin analogue (b) are formulated as separate medicaments.

114. The method of claim 5, wherein said subject is a human.

115. A method for preventing or reducing weight gain; promoting weight loss; improving circulating glucose levels, glucose tolerance or circulating cholesterol levels; lowering circulating LDL levels; increasing HDL/LDL ratio; lowering circulating triacylglycerol levels, lowering circulating free fatty acids or treating a condition caused or characterized by excess body weight in a mammalian subject that is receiving an insulin analogue, said method comprising administering to said subject in an effective amount a compound having the formula:
R¹—Z—R²
wherein R¹ is H, C₁₋₄ alkyl, acetyl, formyl, benzoyl, or trifluoroacetyl;
R² is OH or NH₂;
and Z is a peptide having the formula I:
His-X2-Gln-Gly-Thr-Phe-Thr-Ser-Asp-Tyr-Ser-X12-Tyr-Leu-Asp-X16-X17-Ala-Ala-X20-X21-Phe-Val-X24-Trp-Leu-X27-X28-Ala-X30; (I)
wherein
X2 is selected from Aib and Ser;
X12 is selected from Lys, Arg, or Leu;
X16 is selected from Arg and X;
X17 is selected from Arg and X;
X20 is selected from Arg, His, and X;
X21 is selected from Asp and Glu;
X24 is selected from Ala and X;
X27 is selected from Leu and X;
X28 is selected from Arg and X;
X30 is X or is absent;
wherein at least one of X16, X17, X20, X24, X27, X28, and X30 is X;
and wherein each residue X is independently selected from the group consisting of Glu, Lys, Ser, Cys, Dbu, Dpr, and Orn;
wherein the side chain of at least one residue X is conjugated to a lipophilic substituent having the formula:
(i) Z¹, wherein Z¹ is a lipophilic moiety conjugated directly to the side chain of X; or
(ii) Z¹Z², wherein Z¹ is a lipophilic moiety, Z² is a spacer, and Z¹ is conjugated to the side chain of X via Z².

116. The method of claim 115, wherein said insulin analogue is selected from the group consisting of insulin glulisine (Apidra), insulin lispro (Humalog), Degludec, Actraphane HM, LY2963016, LY2605541, pegylated insulin Lispro, insulin glargine (Lantus), insulin detemir (Levemir), insulin isophane, insulin aspart, insulin buccal, hyaluronidase insulin, insulin protamine, NN-1953, IN-105, BIOD-620 and Analog-PH20.
